(19) United States
(12) Patent Application Publication
(10) Pub. No.: US 2023/0210851 A1
(43) Pub. Date: Jul. 6, 2023

(54) SPIRO-LACTAM COMPOUNDS AND METHODS OF TREATING VIRAL INFECTIONS USING THE SAME

(71) Applicant: Aptinyx Inc., Evanston, IL (US)

(72) Inventor: M. Amin Khan, Evanston, IL (US)

(21) Appl. No.: 18/000,571

(22) PCT Filed: Jun. 3, 2021
(86) PCT No.: PCT/US2021/035724

§ 371 Date: Dec. 2, 2022

Related U.S. Application Data
(60) Provisional application No. 63/133,901, filed on Jan. 5, 2021.
(60) Provisional application No. 63/034,076, filed on Jun. 3, 2020.

(51) Int. Cl. A61K 31/499; A61K 31/5386; A61K 31/675; A61K 47/54; A61P 31/14

(52) U.S. Cl. CPC A61K 31/499; A61K 31/675; A61K 31/5386; A61K 47/542; A61P 31/14

(57) ABSTRACT

Disclosed are spiro-lactam compounds, and pharmaceutically acceptable salts thereof, that can ameliorate or treat a viral infection in a subject in need thereof. The disclosure also includes conjugates of such compounds of viral protease inhibitors with the cysteine at position or an equivalent active site cysteine on the coronavirus main protease (Mpro).

### CROSS-REFERENCE TO RELATED APPLICATION

This application claims the benefit of and priority to U.S. Pat. Application No. 63/133,901, filed Jan. 5, 2021, and U.S. Pat. Application No. 63/034,076, filed Jun. 3, 2020, the contents each of which is incorporated by reference herein in their entirety.

### BACKGROUND

Coronaviruses (CoVs) constitute a group of phylogenetically diverse enveloped viruses that encode the largest plus strand RNA genomes and replicate efficiently in most mammals. Human CoV (HCoVs-229E, OC43, NL63, and HKU1) infections typically result in mild to severe upper and lower respiratory tract disease. Severe Acute Respiratory Syndrome Coronavirus (SARS-CoV) emerged in 2002-2003 causing acute respiratory distress syndrome (ARDS) with 10% mortality overall and up to 50% mortality in aged individuals. Middle Eastern Respiratory Syndrome Coronavirus (MERS-CoV) emerged in the Middle East in April of 2012, manifesting as severe pneumonia, acute respiratory distress syndrome (ARDS) and acute renal failure. More recently, COVID-19 (SARS CoV2) coronaviruses have raised a global pandemic since they had been first identified in China in late 2019.

One of the best-characterized drug targets among coronaviruses is the main protease (Mpro, also called 3CLpro). Along with the papain-like protease(s), this enzyme is essential for processing the polyproteins that are translated from the viral RNA. These proteases process the CoV replicase polyprotein by cleaving it into 16 non-structural proteins, which are responsible for a variety of aspects of CoV replication. The CoV Mpro is responsible for processing 11 cleavage sites of within the replicase polyprotein and is essential for CoV replication, making it a highly valuable target for therapeutic development. The overall active site architecture and substrate recognition pockets are structurally conserved across CoV Mpros, increasing its attractiveness as a target for the development of broad-spectrum anti-CoV therapeutics. Moreover, high sequence conservation in the vicinity of active site among CoV Mpros from different coronavirus subclasses make them an excellent target for the development of broad-spectrum therapeutics for coronavirus infections. Accordingly, the development of CoV Mpro inhibitors is a promising path for the treatment of respiratory tract infections and related diseases.

The coronavirus infection is a continuing threat to the human health and has high fatality rate. The virus also demonstrates person-to-person transmission, posing a continuous threat to public health worldwide. Therefore, there is a critical need for preventive and therapeutic antiviral agents for the treatment of coronavirus infections.

### SUMMARY

Described herein are compounds, for example, spiro-lactam compounds, that can be useful in methods of ameliorating or treating a viral infection in a subject in need thereof. The present disclosure should be understood to include compounds as described herein as well as methods of using the compounds for treatment of viral infections. The present disclosure also includes other aspects of the inventions described herein such as conjugates. Each of these different aspects can be described more particularly by the various embodiments described herein, which embodiments can be equally applicable to the different aspects.

The compounds include those of Formula (A) and the various subgenuses thereof as described herein. The methods generally comprise administering to the subject a therapeutically effective amount of a compound of Formula (A), or a pharmaceutically acceptable salt and/or a stereoisomer thereof, wherein Formula (A) is:

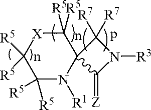

wherein:
X is O or NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ and R³² are each independently selected from the group consisting of H, C₁—C₆alkyl, —C₃—C₆cycloalkyl, and phenyl, wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano and halogen;
R⁷ is independently selected for each occurrence from the group consisting of H, C₁-C₆ alkyl, phenyl and halogen;
R^{a} and R^{b} are each independently for each occurrence selected from the group consisting of H, C₁-C₃alkyl, and phenyl, or R^{a} and R^{b} taken together with the nitrogen to which they are attached form a 4-6 membered heterocyclic ring;
p is 1 or 2;
n is independently, for each occurrence, 0, 1 or 2; and
w is independently, for each occurrence, 0, 1 or 2.

In various embodiments, Formula (A), or a pharmaceutically acceptable salt and/or a stereoisomer thereof, is:

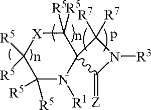

wherein:
X is O or NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ and R³² are each independently selected from the group consisting of H, C₁—C₆alkyl, —C₃—C₆cycloalkyl, and phenyl, wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl, halogen and —O—P(O)(R⁴¹R⁴²); and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl, and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl);
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano, and halogen;
R⁷ is independently selected for each occurrence from the group consisting of H, C₁—C₆ alkyl, phenyl, and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 1 or 2;
n is independently, for each occurrence, 0, 1 or 2; and
w is independently, for each occurrence, 0, 1 or 2.

In some embodiments, Formula (A), or a pharmaceutically acceptable salt and/or a stereoisomer thereof, is:

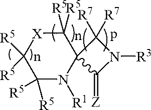

wherein:
X is NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of —C(O)R³¹, —C(S)R³¹, —C(NH)R³¹ and —C(O)OR³²;
R³ is selected from the group consisting of H, C₁-C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ is C₁—C₆alkyl, wherein C₁—C₆ alkyl is substituted by one, two or three substituents each independently selected from hydroxyl, S(O)₂—C₁—C₃alkyl, halogen and —O—P(O)(R⁴¹R⁴²);
R³² is C₁—C₆alkyl;
R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl);
R⁵ is independently selected for each occurrence from the group consisting of H, C₁—C₆alkyl, —C₁—C₃alkoxy, —S(O)_{w}—C₁—C₃alkyl, - NR^{a}R^{b}, cyano and halogen;
R⁷ is independently selected for each occurrence from the group consisting of H, C₁—C₆ alkyl, phenyl and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 2;
n is, for each occurrence, 1; and
w is independently, for each occurrence, 0, 1 or 2.

A compound of the present disclosure, or its pharmaceutically acceptable salt, can also be referred to herein as a “viral protease inhibitor” or “VPI,” which can include a C=Z′ moiety, wherein Z′ is O, S or NH.

In some embodiments, for the compound of Formula (A), R⁵, at each occurrence, is H.

In some embodiments, for the compound of Formula (A), R⁷, at each occurrence, is H.

In some embodiments, for the compound of Formula (A), at least one of R¹, R² and R³, independently is —C(O)(C₁—C₆alkyl)X′, wherein X′ is a halogen.

In some embodiments, for the compound of Formula (A), at least one of R¹, R² and R³, independently is —C(O)(CH)(CH₃)X′, wherein X′ is a halogen.

In some embodiments, for the compound of Formula (A), at least one of R¹, R² and R³, independently is —C(O)(C₁—C₆alkyl)X′, wherein X′ is —O—P(O)(R⁴¹R⁴²), wherein R⁴¹ is selected from —O(C₁—C₆alkyl) and —O—phenyl, and R⁴² is —NH(C₁—C₆alkyl) optionally substituted by —C(O)—O(C₁—C₆alkyl).

In some embodiments, for the compound of Formula (A), Z is O.

In some embodiments, for the compound of Formula (A), X′ is Br, Cl, or F.

In some embodiments, for the compound of Formula (A), X′ is Br, Cl, F, or I.

In some embodiments, for the compound of Formula (A), X′ is —O—P(O)(R⁴¹R⁴²), wherein R⁴¹ is selected from —O(C₁—C₆alkyl) and —O—phenyl, and R⁴² is —NH(C₁—C₆alkyl) optionally substituted by —C(O)—O(C₁—C₆alkyl).

In some embodiments, for the compound of Formula (A), X′ is selected from the group consisting of

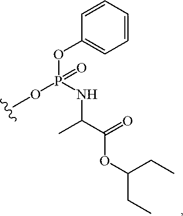

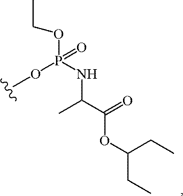

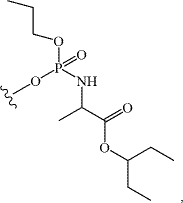

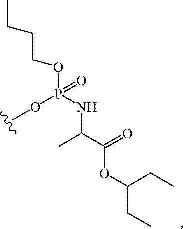

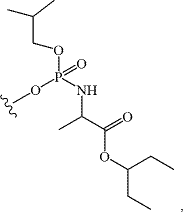

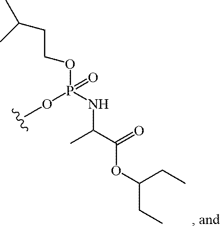

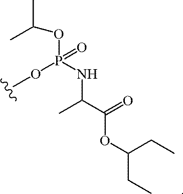

In some embodiments, for the compound of Formula (A), n, for each occurrence is 1.

In some embodiments, for the compound of Formula (A), p is 1.

In some embodiments, for the compound of Formula (A), R¹ is H.

In certain embodiments, for the compound of Formula (A), R¹ is —(CH₂)—phenyl, wherein the phenyl may optionally be substituted by one, two or three halogen.

In some embodiments, for the compound of Formula (A), X is NR².

In some embodiments, for the compound of Formula (A), R² is —C(O)(C₁—C₆alkyl)X′, wherein X′ is a halogen.

In some embodiments, for the compound of Formula (A), R² is —C(O)(C₁—C₆alkyl)X′, wherein X′ is —O—P(O)(R⁴¹R⁴²), wherein R⁴¹ is selected from —O(C₁—C₆alkyl) and —O—phenyl, and R⁴² is —NH(C₁—C₆alkyl) optionally substituted by —C(O)—O(C₁—C₆alkyl).

In certain embodiments, for the compound of Formula (A), R³ is C₁—C₂alkyl, optionally substituted by one or two substituents each independently selected from phenyl and halogen; and phenyl, independently for each occurrence, is optionally substituted by one, two or three substituents each independently selected from hydroxyl, halogen, —C(O)—C₁—C₃alkyl, methyl, and CF₃.

In other embodiments, for compound of Formula (A), R³ is —CH2—phenyl, wherein phenyl is optionally substituted by one, two or three substituents each independently selected from hydroxyl, halogen, —C(O)—C₁—C₃alkyl, methyl, and CF₃.

In other embodiments, for compound of Formula (A), R³ is H.

In certain embodiments, for compound of Formula (A), Formula (A) is:

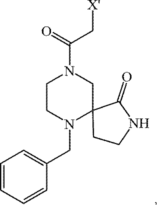

wherein X′ is Br, Cl, or F.

In certain embodiments, Formula (A) is:

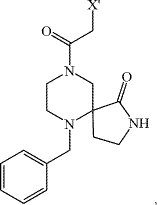

wherein X′ is I or —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl).

In some embodiments, for compound of Formula (A), X′ is selected from the group consisting of

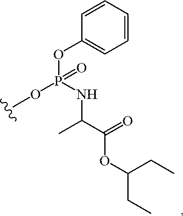

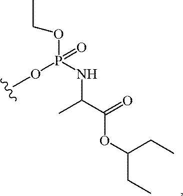

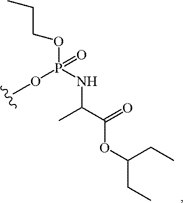

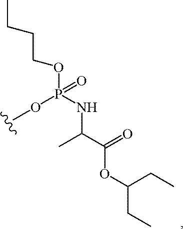

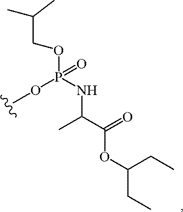

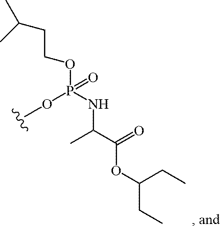

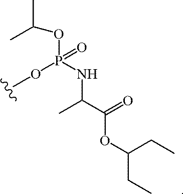

In some embodiments, the compound of Formula (A) is a compound having Formula (A-I):

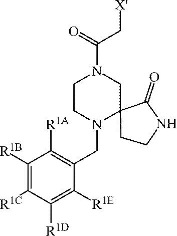

wherein:
X′ is a halogen; and
one, two or three of R^{1A}, R^{1B}, R^{1C}, R^{1D}, and R^{1E} are optionally each independently selected from the group consisting of hydroxyl, halogen, —C(O)—O—C₁—C₃alkyl, —C(O)—C₁—C₃alkyl, methyl, and CF₃.

In some embodiments, for Formula (A-I), the compound is selected from the group consisting of a compound having Formula (A-II); a compound having Formula (A-III); and a compound having Formula (A-IV), wherein:

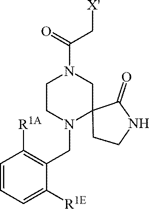

wherein:
X′ is a halogen; and
R^{1A} and R^{1E} are optionally each independently selected from the group consisting of hydroxyl, halogen, —C(O)—O—C₁—C₃alkyl, —C(O)—C₁—C₃alkyl, methyl, and CF₃; Formula (A-III) is:

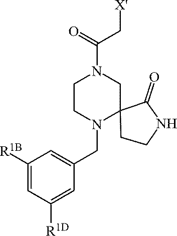

wherein:
   X′ is a halogen; and
   R^{1B} and R^{1D} are optionally each independently selected from the group consisting of hydroxyl, halogen, —C(O)—O—C₁—C₃alkyl, —C(O)—C₁—C₃alkyl, methyl, and CF₃; and Formula (A-IV) is: 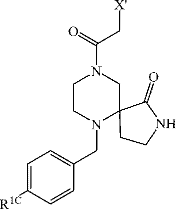
   wherein:
      X′ is a halogen; and
      R^{1C} is optionally selected from the group consisting of hydroxyl, halogen, —C(O)—O—C₁—C₃alkyl, —C(O)—C₁—C₃alkyl, methyl, and CF₃.

In some embodiments, for Formula (A-I), the compound is selected from the group consisting of a compound having Formula (A-V); and a compound having Formula (A-VI), wherein: Formula (A-V) is:

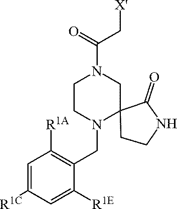

wherein:
X′ is a halogen; and
R^{1A}, R^{1C}, and R^{1E} are optionally each independently selected from the group consisting of hydroxyl, halogen, —C(O)—O—C₁—C₃alkyl, —C(O)—C₁—C₃alkyl, methyl, and CF₃; and Formula (A-VI) is:

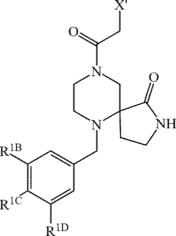

wherein:
   X′ is a halogen; and
   R^{1B}, R^{1C}, and R^{1D} are optionally each independently selected from the group consisting of hydroxyl, halogen, —C(O)—O—C₁—C₃alkyl, —C(O)—C₁—C₃alkyl, methyl, and CF₃.

In some embodiments, the compound of Formula (A) is a compound having Formula (A-I):

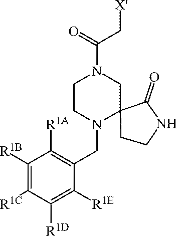

wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH2, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
one, two or three of R^{1A}, R^{1B}, R^{1C}, R^{1D}, and R^{1E} are optionally each independently selected from the group consisting of H, hydroxyl, halogen, —C(O)—O—C₁—C₃alkyl, —C(O)—C₁—C₃alkyl, methyl, and CF₃.

In some embodiments, for Formula (A-I), the compound is selected from the group consisting of a compound having Formula (A-II); a compound having Formula (A-III); and a compound having Formula (A-IV), wherein:
Formula (A-II) is:

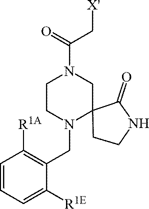

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
   R^{1A} and R^{1E} are optionally each independently selected from the group consisting of hydroxyl, halogen, —C(O)—O—C₁—C₃alkyl, —C(O)—C₁—C₃alkyl, methyl, and CF₃;
Formula (A-III) is:

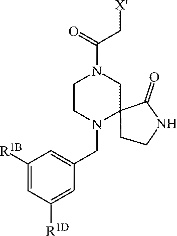

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁-C₆alkyl, -C₃-C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
   R^{1B} and R^{1D} are optionally each independently selected from the group consisting of hydroxyl, halogen, —C(O)—O—C₁—C₃alkyl, —C(O)—C₁—C₃alkyl, methyl, and CF₃; and Formula (A-IV) is: 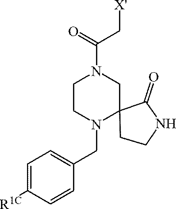
   wherein:
      X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH2, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
      R^{1C} is optionally selected from the group consisting of hydroxyl, halogen, —C(O)—O—C₁—C₃alkyl, —C(O)—C₁—C₃alkyl, methyl, and CF₃.

In some embodiments, for Formula (A-I), for Formula (A-I), the compound is selected from the group consisting of a compound having Formula (A-V); and a compound having Formula (A-VI), wherein:
Formula (A-V) is:

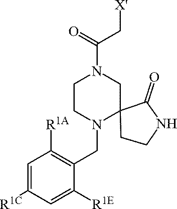

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
   R^{1A}, R^{1C}, and R^{1E} are optionally each independently selected from the group consisting of hydroxyl, halogen, —C(O)—O—C₁—C₃alkyl, —C(O)—C₁—C₃alkyl, methyl, and CF₃; and Formula (A-VI) is: 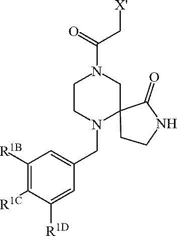
   wherein:
      X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and -N(C₁-C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
      R^{1B}, R^{1C}, and R^{1D} are optionally each independently selected from the group consisting of hydroxyl, halogen, —C(O)—O—C₁—C₃alkyl, —C(O)—C₁—C₃alkyl, methyl, and CF₃.

In some embodiments, the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenovirus, a herpes virus, and a hepatovirus.

In some embodiments, the viral infection is a coronavirus infection.

Also described herein are conjugates, which can be reversible conjugates, represented by:

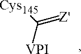

wherein Cys₁₄₅ is cysteine at position 145 or equivalent active site cysteine on Mpro, for example, a CoV Mpro; Z′ is O, S or NH; and VPI is a viral protease inhibitor.

In some embodiments, the conjugate is represented by:

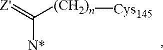

wherein:
Cys₁₄₅ is cysteine at position 145 or equivalent active site cysteine on Mpro, for example, a CoV Mpro;
Z′ is O, S or NH;
n is independently, for each occurrence, 0, 1 or 2; and
N* is a ring nitrogen of a compound, or a pharmaceutically acceptable salt and/or a stereoisomer thereof, wherein N* comprises the compound, or a pharmaceutically acceptable salt and/or a stereoisomer thereof, wherein the compound is a compound having Formula (A).

In certain embodiments, the conjugate is represented by:

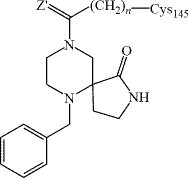

wherein Z′ is O, S or NH; and
n is independently, for each occurrence, 0, 1 or 2.

In embodiments, Z′ is O.

In embodiments, n is 1.

In certain embodiments, the conjugate is represented by:

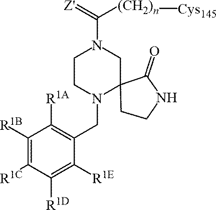

wherein Z′ is O, S or NH;
n is independently, for each occurrence, 0, 1 or 2; and
one, two or three of R^{1A}, R^{1B}, R^{1C}, R^{1D}, and R^{1E} are optionally each independently selected from the group consisting of H, hydroxyl, halogen, —C(O)—O—C₁—C₃alkyl, —C(O)—C₁—C₃alkyl, methyl, and CF₃.

### BRIEF DESCRIPTION OF FIGURES

FIG. 1. depicts images of superimposed crystal structures of two piperazine fragments, PDB: 5REL and 5RG0, with and without the carboxyl linker group (top and middle), and the chemical structure of ES-319/320 (bottom).

FIG. 2 depicts a structural design scheme for ES-319/320.

FIG. 3 depicts an image of the 2D interaction view of ES-319 on the active site of SARS-CoV2 (PDB: 5REL) crystal structure generated using Covalent docking studies (Schrodinger Suite).

FIG. 4 depicts an image of a proposed mechanism for covalent binding of ES-319/320 analogue with SARS-CoV2.

FIG. 5 depicts an image of the 3D interaction view of ES-319 on the active site of SARS-CoV2 (PDB: 5REL) crystal structure generated using Covalent Docking Studies (Schrodinger Suite).

FIG. 6 depicts IC₅₀ curves of GC376 that were obtained using the 3CL Protease (SARS-CoV-2) Assay of the present disclosure.

FIG. 7 depicts IC₅₀ curves of GC376 disclosed in Vuong, W., et al. Nat Commun 11, 4282 (2020).

FIG. 8 depicts an IC₅₀ curve of GC376 disclosed by BPS Biosciences.

FIG. 9 depicts % inhibitory activity of ET-103 (top left), ET-319 (top right), ES-320 (bottom left), and GC376 (bottom right) that were obtained using the 3CL Protease (SARS-CoV-2) Assay of the present disclosure.

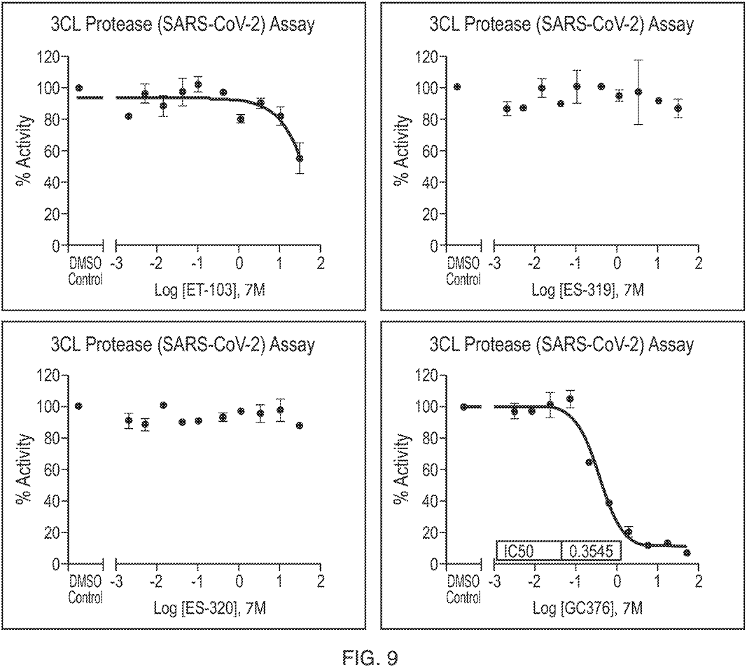

### DETAILED DESCRIPTION

The present disclosure is generally directed to compounds, and pharmaceutically acceptable salts thereof, that are capable of ameliorating or treating a viral infection in a subject in need thereof. More specifically, the present disclosure is directed to methods of ameliorating or treating a viral infection in a subject in need thereof, comprising administering to the subject a therapeutically effective amount of a compound, or a pharmaceutically acceptable salt and/or a stereoisomer thereof, wherein the compound is a compound having Formula (A), as disclosed herein. The present disclosure is also related to conjugates, e.g., reversible conjugates, including the compounds of the present disclosure.

The term “alkyl,” as used herein, refers to a saturated straight-chain or branched hydrocarbon, such as a straight-chain or branched group of 1-6, 1-4, or 1-3 carbon atoms, referred to herein as C₁—C₆ alkyl, C₁—C₄ alkyl, and C₁—C₃ alkyl, respectively. For example, “C₁—C₆ alkyl” refers to a straight-chain or branched saturated hydrocarbon containing 1-6 carbon atoms. Examples of a C₁—C₆ alkyl group include, but are not limited to, methyl, ethyl, propyl, butyl, pentyl, hexyl, isopropyl, isobutyl, sec-butyl, tert-butyl, isopentyl, and neopentyl. In another example, “C₁—C₄ alkyl” refers to a straight-chain or branched saturated hydrocarbon containing 1-4 carbon atoms. Examples of a C₁—C₄ alkyl group include, but are not limited to, methyl, ethyl, propyl, butyl, isopropyl, isobutyl, sec-butyl and tert-butyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

The term “alkoxy,” as used herein, refers to an alkyl group attached to an oxygen atom (alkyl—O—). Alkoxy groups can have 1-6 or 2-6 carbon atoms and are referred to herein as C₁—C₆ alkoxy and C₂—C₆ alkoxy, respectively. Exemplary alkoxy groups include, but are not limited to, methoxy, ethoxy, propyloxy, isopropoxy, and tert-butoxy.

The terms “aryl” and “heteroaryl,” as used herein, refer to mono- or polycyclic unsaturated moieties having preferably 3-14 carbon atoms, each of which may be substituted or unsubstituted. In certain embodiments, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments, “heteroaryl” refers to a mono- or bicyclic heterocyclic ring system having one or two aromatic rings in which one, two, or three ring atoms are heteroatoms independently selected from the group consisting of S, O, and N and the remaining ring atoms are carbon. Non-limiting examples of heteroaryl groups include pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

The term “carbonyl,” as used herein, refers to the radical —C(O)— or C═O.

The term “cyano,” as used herein, refers to the radical —CN.

The term “cycloalkyl,” as used herein, refers to a monocyclic saturated or partially unsaturated hydrocarbon ring (carbocyclic) system, for example, where each ring is either completely saturated or contains one or more units of unsaturation, but where no ring is aromatic. A cycloalkyl can have 3-6 or 4-6 carbon atoms in its ring system, referred to herein as C₃—C₆ cycloalkyl or C₄—C₆ cycloalkyl, respectively. Exemplary cycloalkyl groups include, but are not limited to, cyclohexyl, cyclohexenyl, cyclopentyl, cyclopentenyl, cyclobutyl, and cyclopropyl.

The phrase, “carbocyclic ring,” as used herein, refers to a hydrocarbon ring system in which all the ring atoms are carbon. Exemplary carbocyclic rings including cycloalkyls and phenyl.

The terms “halo” and “halogen,” as used herein, refer to fluoro (F), chloro (Cl), bromo (Br), and/or iodo (I).

The term “haloalkyl” as used herein refers to an alkyl group substituted with one or more halogen atoms.

The term “heteroatom,” as used herein, refers to an atom of any element other than carbon or hydrogen and includes, for example, nitrogen (N), oxygen (O), silicon (Si), sulfur (S), phosphorus (P), and selenium (Se).

The term “heterocyclic ring” or “heterocycloalkyl,” as used herein, is art-recognized and refer to saturated or partially unsaturated 3- to 8-membered ring structures, whose ring system include one, two or three heteroatoms, such as nitrogen, oxygen, and/or sulfur. A heterocyclic ring can be fused to one or more phenyl, partially unsaturated, or saturated rings. Examples of heterocyclic rings include, but are not limited to, pyrrolidinyl, piperidinyl, morpholinyl, thiomorpholinyl, and piperazinyl.

The terms “hydroxy” and “hydroxyl,” as used herein, refer to the radical —OH.

The term “oxo,” as used herein, refers to the radical ═O (double bonded oxygen).

The term “compound,” as used herein, refers to the compound itself and its pharmaceutically acceptable salts, hydrates, esters and N-oxides including its various stereoisomers and its isotopically-labelled forms, unless otherwise understood from the context of the description or expressly limited to one particular form of the compound, i.e., the compound itself, a specific stereoisomer and/or isotopically-labelled compound, or a pharmaceutically acceptable salt, a hydrate, an ester, or an N-oxide thereof. It should be understood that a compound can refer to a pharmaceutically acceptable salt, or a hydrate, an ester or an N-oxide of a stereoisomer of the compound and/or an isotopically-labelled compound.

The compounds of the disclosure can contain one or more chiral centers and/or double bonds and therefore, can exist as stereoisomers, such as geometric isomers, and enantiomers or diastereomers. The term “stereoisomers,” when used herein, consists of all geometric isomers, enantiomers and/or diastereomers of the compound. For example, when a compound is shown with specific chiral center(s), the compound depicted without such chirality at that and other chiral centers of the compound are within the scope of the present disclosure, i.e., the compound depicted in two-dimensions with “flat” or “straight” bonds rather than in three dimensions, for example, with solid or dashed wedge bonds. Stereospecific compounds may be designated by the symbols “R” or “S,” depending on the configuration of substituents around the stereogenic carbon atom. The present disclosure encompasses all the various stereoisomers of these compounds and mixtures thereof. Mixtures of enantiomers or diastereomers can be designated “(±)” in nomenclature, but a skilled artisan will recognize that a structure can denote a chiral center implicitly. It is understood that graphical depictions of chemical structures, e.g., generic chemical structures, encompass all stereoisomeric forms of the specified compounds, unless indicated otherwise.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their component enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. See, for example, Carreira and Kvaerno, Classics in Stereoselective Synthesis, Wiley-VCH: Weinheim, 2009.

Geometric isomers, resulting from the arrangement of substituents around a carbon-carbon double bond or arrangement of substituents around a cycloalkyl or heterocycloalkyl, can also exist in the compounds of the present disclosure. The symbol denotes a bond that may be a single, double or triple bond as described herein. Substituents around a carbon-carbon double bond are designated as being in the “Z” or “E′ configuration, where the terms “Z” and “E′ are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The disclosure also embraces isotopically-labeled compounds which are identical to those compounds recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H (“D”), ³H, ¹³C, ¹⁴C, ¹⁵N, ¹⁸O, ¹⁷O, ³¹P, ³²P, ³⁵S, ¹⁸F, and ³⁶C1, respectively. For example, a compound described herein can have one or more H atoms replaced with deuterium.

Certain isotopically-labeled compounds (e.g., those labeled with ³H and ¹⁴C) can be useful in compound and/or substrate tissue distribution assays. Tritiated (i.e., ³H) and carbon-14 (i.e., ¹⁴C) isotopes can be particularly preferred for their ease of preparation and detectability. Further, substitution with heavier isotopes such as deuterium (i.e., ²H) can afford certain therapeutic advantages resulting from greater metabolic stability (e.g., increased in vivo half-life or reduced dosage requirements) and hence can be preferred in some circumstances. Isotopically-labeled compounds can generally be prepared by following procedures analogous to those disclosed herein, for example, in the Examples section, by substituting an isotopically-labeled reagent for a non-isotopically-labeled reagent.

The phrases “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, general safety and purity standards as required by FDA Office of Biologics standards.

The phrases “pharmaceutically acceptable carrier” and “pharmaceutically acceptable excipient,” as used herein, refer to any and all solvents, dispersion media, coatings, isotonic and absorption delaying agents, and the like, that are compatible with pharmaceutical administration. Pharmaceutical acceptable carriers can include phosphate buffered saline solution, water, emulsions (e.g., such as an oil/water or water/oil emulsions), and various types of wetting agents. The compositions also can include stabilizers and preservatives.

The phrase “pharmaceutical composition,” as used herein, refers to a composition comprising at least one compound as disclosed herein formulated together with one or more pharmaceutically acceptable carriers. The pharmaceutical compositions can also contain other active compounds providing supplemental, additional, or enhanced therapeutic functions.

The terms “individual,” “patient,” and “subject,” as used herein, are used interchangeably and include any animal, including mammals, preferably mice, rats, other rodents, rabbits, dogs, cats, swine, cattle, sheep, horses, or primates, and more preferably, humans. The compounds described in the disclosure can be administered to a mammal, such as a human, but can also be administered to other mammals such as an animal in need of veterinary treatment, for example, domestic animals (e.g., dogs, cats, and the like), farm animals (e.g., cows, sheep, pigs, horses, and the like) and laboratory animals (e.g., rats, mice, guinea pigs, and the like). The mammal treated in the methods described in the disclosure is preferably a mammal in which treatment, for example, of pain or depression, is desired.

The term “treating,” as used herein, includes any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The term “disorder” refers to and is used interchangeably with, the terms “disease,” “condition,” or “illness,” unless otherwise indicated.

The phrase “therapeutically effective amount,” as used herein, refers to the amount of a compound (e.g., a disclosed compound) that will elicit the biological or medical response of a tissue, system, animal or human that is being sought by the researcher, veterinarian, medical doctor or other clinician. The compounds described in the disclosure can be administered in therapeutically effective amounts to treat a disease. A therapeutically effective amount of a compound can be the quantity required to achieve a desired therapeutic and/or prophylactic effect, such as an amount which results in lessening of a symptom of a disease such as depression.

As used herein, the term “pharmaceutically acceptable salt” refers to any salt of an acidic or a basic group that may be present in a compound of the present disclosure, which salt is compatible with pharmaceutical administration. As is known to those of skill in the art, “salts” of the compounds of the present disclosure may be derived from inorganic or organic acids and bases.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH₄⁺, and NW₄+ (where W can be a C₁₋₄ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

The compounds disclosed herein can exist in a solvated form as well as an unsolvated form with pharmaceutically acceptable solvents such as water, ethanol, and the like, and it is intended that the disclosure embrace both solvated and unsolvated forms.

Unless defined otherwise, all technical and scientific terms used herein have the same meaning as commonly understood by one of ordinary skill in the art to which this disclosure pertains.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments can be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also include the specific quantitative value itself, unless specifically stated otherwise. As used herein, the term “about” refers to a ±10% variation from the nominal value unless otherwise indicated or inferred.

Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁—C₆, C₁—C₅, C₁—C₄, C₁—C₃, C₁—C₂, C₂—C₆, C₂—C₅, C₂—C₄, C₂—C₃, C₃—C₆, C₃—C₅, C₃—C₄, C₄—C₆, C₄—C₅, and C₅—C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Further, if a variable is not accompanied by a definition, then the variable is defined as found elsewhere in the disclosure unless understood to be different from the context. In addition, the definition of each variable and/or substituent, for example, C₁—C₆ alkyl, R², R^{b}, w and the like, when it occurs more than once in any structure or compound, can be independent of its definition elsewhere in the same structure or compound.

Definitions of the variables and/or substituents in formulae and/or compounds herein encompass multiple chemical groups. The present disclosure includes embodiments where, for example, i) the definition of a variable and/or substituent is a single chemical group selected from those chemical groups set forth herein, ii) the definition is a collection of two or more of the chemical groups selected from those set forth herein, and iii) the compound is defined by a combination of variables and/or substituents in which the variables and/or substituents are defined by (i) or (ii).

In certain embodiments, R¹, R², and/or R³ independently can be an amino acid or a derivative of an amino acid, for example, an alpha “amino amide” represented by H₂N—CH (amino acid side chain)—C(O)NH₂. In certa In certain embodiments, the nitrogen atom of the amino group of the amino acid or the amino acid derivative is a ring nitrogen in a chemical formula described herein.in embodiments, the nitrogen atom of the amino group of the amino acid or the amino acid derivative is a ring nitrogen in a chemical formula described herein. In such embodiments, the carboxylic acid of the amino acid or the amide group of an amino amide (amino acid derivative) is not within the ring structure, i.e., not a ring atom. In certain embodiments, the carboxylic acid group of the amino acid or the amino acid derivative forms an amide bond with a ring nitrogen in a chemical formula disclosed herein, thereby providing an amino amide, where the amino group of the amino amide is not within the ring structure, i.e., not a ring atom. In certain embodiments, R¹, R², and/or R³ independently can be an alpha amino acid, an alpha amino acid derivative, and/or another amino acid or amino acid derivative such as a beta amino acid or a beta amino acid derivative, for example, a beta amino amide.

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

### Compounds

It has now been discovered that compounds of the present disclosure, and pharmaceutically acceptable salts thereof, can bind to, dock with, and/or inhibit a viral protease, for example, Mpro, to ameliorate or treat a viral infection. In particular, the crystal structure of the SARS-CoV2 main protease (MPro or CoV Mpro) was determined, with about 68 crystal structures of MPro complexed with fragments reported. Of the 68 crystal structures, 22 crystal structures are complexed with non-covalent interactions, and 44 crystal structures are complexed with fragments with covalent bonding.

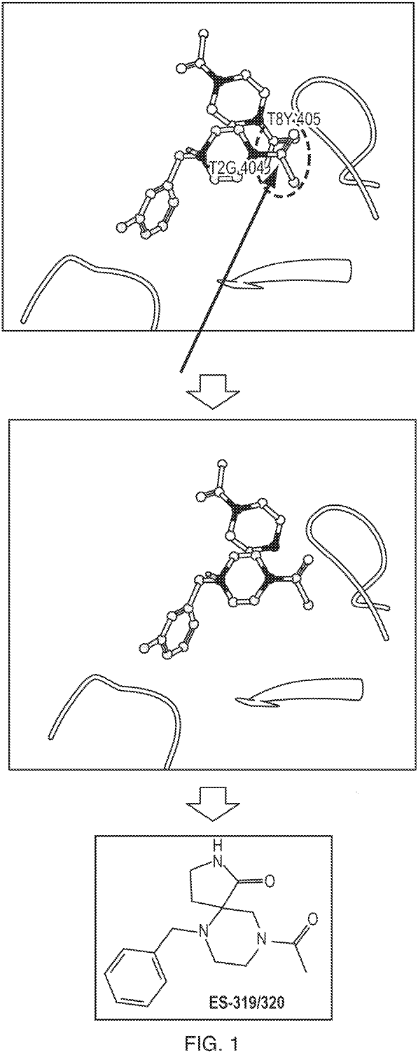

A superimposition of two crystal structures of two piperazine fragments, PDB: 5REL and 5RG0, are depicted in FIG. 1. The superimposition shows that two co-crystals (fragments) bind in slightly different orientation and share similar interactions. The top image of FIG. 1 depicts that the carboxyl group of both fragments share the same space and interactions, and the middle image of FIG. 1 depicts a carboxyl linker removed from the fragments. The images suggest that spiro cyclic (6,6; 6,5) or bicyclic groups can be useful as COVID inhibitors. The two piperazine fragments, PDB: 5REL and 5RG0, are very similar to compounds of the present disclosure.

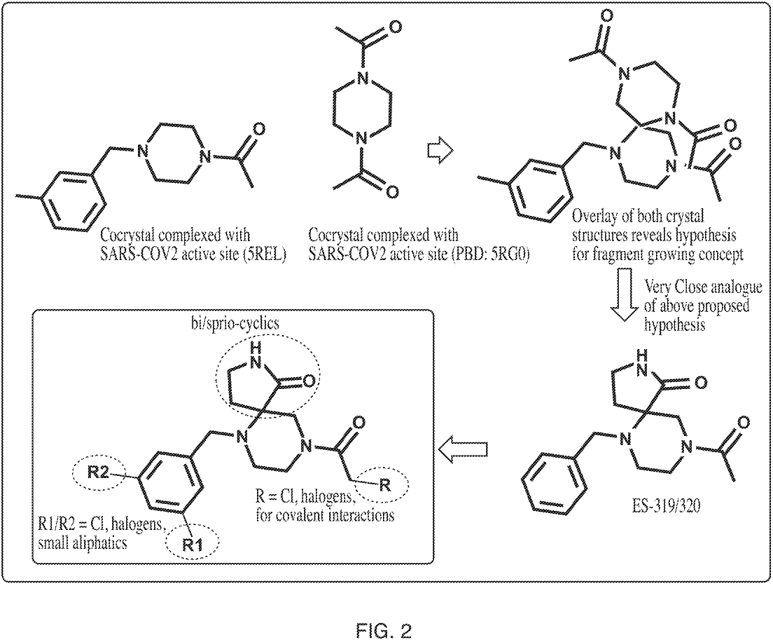

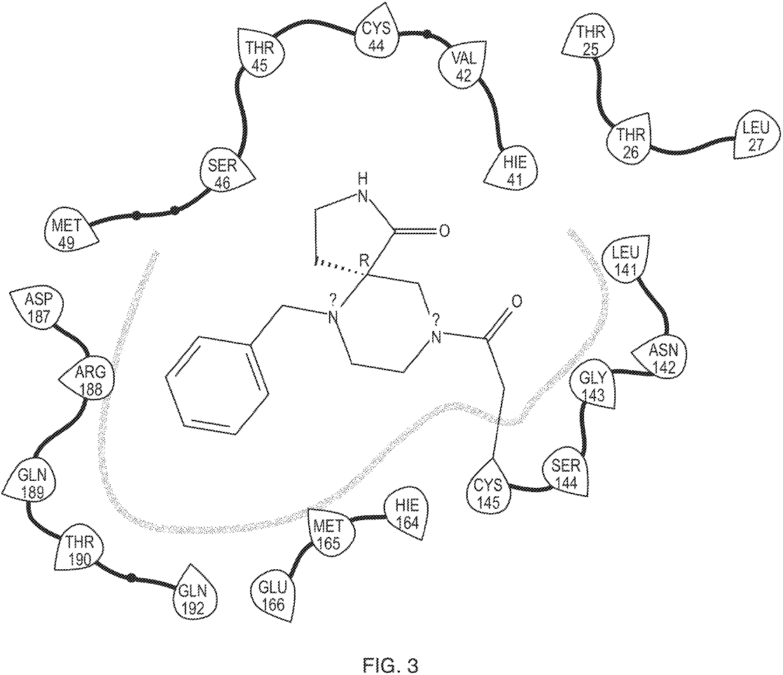

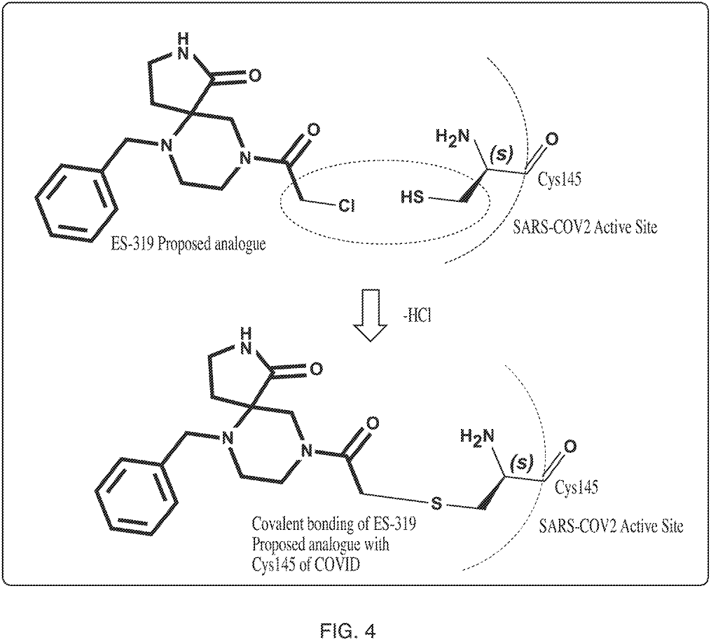

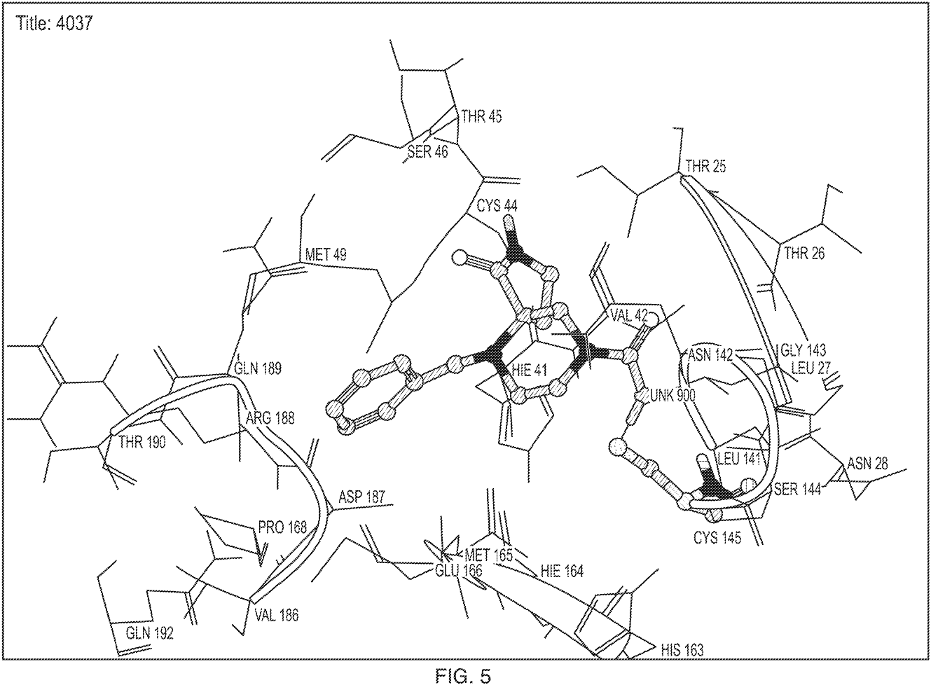

Covalent docking studies of ES-319 with the SARS-CoV2 was carried out to understand the binding mode of ES-319. A scheme that depicts the design concept of ES-319/320 is shown in FIG. 2. The scheme illustrates the structural similarity between an overlay of two cocrystal structures (PDB: 5RG0 and 5REL) and ES-319/320, as well as positions in ES-319/320 available for functionalization. Furthermore, images of 2D (FIG. 3) and 3D (FIG. 5) interaction views of ES-319 covalently bound to Cys145 of SARS-CoV2 were obtained using Covalent docking studies. As depicted in FIG. 4, a proposed mechanism for covalent binding of ES-319/320 analogues with a SARS-CoV2 active site involves a nucleophilic substitution with a chloro group of ES-319/320 and the thiol of SARS-CoV2 active site (Cys145) to form a covalent bond.

Thus, the docking studies revealed that ES-319 shows good affinity for and can covalently bind with Cys145, suggesting that ES-319 analogues are promising as COVID inhibitors.

Based on the above, a compound or a pharmaceutically acceptable salt thereof, useful in the methods of the present disclosure can include a compound having Formula (A), as described herein.

### Compounds of Formula (A)

In some embodiments, the methods and conjugates described herein use compounds of Formula (A), or a pharmaceutically acceptable salt and/or a stereoisomer thereof, wherein Formula (A) is:

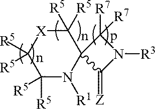

wherein:
X is O or NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of H, C₁—C₆alkyl, phenyl, —C(O)R³¹, —C(S)R³¹, —C(NH)R³¹ and —C(O)OR³², wherein C₁-C₆ alkyl is optionally substituted by one, two or three substituents each independently selected from —C(O)NR^{a}R^{b}, —NR^{a}R^{b}, hydroxyl, S(O)_{W}-C₁-C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ and R³² are each independently selected from the group consisting of H, C₁—C₆alkyl, —C₃—C₆cycloalkyl, and phenyl, wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano and halogen;
R⁷ is independently selected for each occurrence from the group consisting of H, C₁—C₆ alkyl, phenyl and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 1 or 2;
n is independently, for each occurrence, 0, 1 or 2; and
w is independently, for each occurrence, 0, 1 or 2.

In some embodiments, the compounds of the present disclosure of include the compound of Formula (A), or a pharmaceutically acceptable salt and/or a stereoisomer thereof, wherein Formula (A) is:

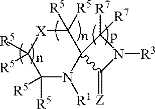

wherein:
X is O or NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ and R³² are each independently selected from the group consisting of H, C₁—C₆alkyl, —C₃—C₆cycloalkyl, and phenyl, wherein C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl, halogen and —O—P(O)(R⁴¹R⁴²); and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl, and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁-C₆alkoxy, —C(O)—(C₁—C₆ alkyl), and —C(O)—O(C₁—C₆alkyl);
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano, and halogen;
R⁷ is independently selected for each occurrence from the group consisting of H, C₁—C₆ alkyl, phenyl, and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 1 or 2;
n is independently, for each occurrence, 0, 1 or 2; and
w is independently, for each occurrence, 0, 1 or 2.

In some embodiments, the compounds of the present disclosure of include the compounds of Formula (A), wherein Formula (A) is:

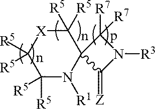

wherein:
Xis NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of —C(O)R³¹, —C(S)R³¹, —C(NH)R³¹ and —C(O)OR³²;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ is C₁—C₆alkyl, wherein C₁—C₆ alkyl is substituted by one, two or three substituents each independently selected from hydroxyl, S(O)₂—C₁—C₃alkyl, halogen and —O—P(O)(R⁴¹R⁴²);
R³² is C₁—C₆alkyl;
R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl);
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano and halogen;
R⁷ is independently selected for each occurrence from the group consisting of H, C₁—C₆ alkyl, phenyl and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 2;
n is, for each occurrence, 1; and
w is independently, for each occurrence, 0, 1 or 2.

In certain embodiments, R⁵, at each occurrence, is H.

In certain embodiments, R⁷, at each occurrence, is H.

In some embodiments, R¹, R² and R³, independently is -C(O)(C₁-C₆alkyl)X′, wherein X′ is a halogen.

In some embodiments, R¹, R² and R³, independently is —C(O)(CH)(CH₃)X′, wherein X′ is a halogen.

In certain embodiments, Z is O.

In some embodiments, X′ is Br, Cl, or F.

In some embodiments, X′ is Br, Cl, F, or I.

In some embodiments, X′ is —O—P(O)(R⁴¹R⁴²), wherein R⁴¹ is selected from —O(C₁—C₆alkyl) and —O—phenyl, and R⁴² is —NH(C₁—C₆alkyl) optionally substituted by —C(O)—O(C₁—C₆alkyl).

In some embodiments, X′ is selected from the group consisting of:

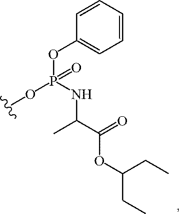

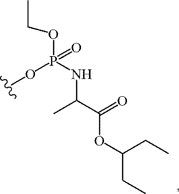

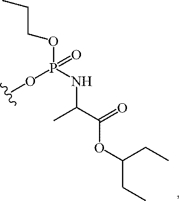

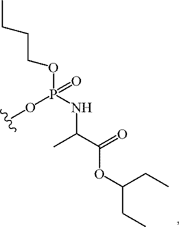

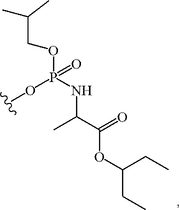

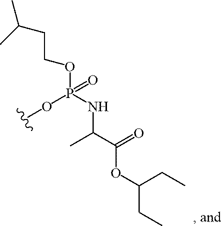

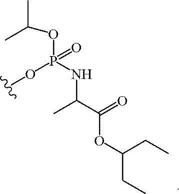

In some embodiments, n, for each occurrence is 1.

In certain embodiments, p is 1.

In certain embodiments, R¹ is —(CH₂)—phenyl, wherein the phenyl may optionally be substituted by one, two or three halogen.

In some embodiments, R¹ is C₁—C₆alkyl, C₁—C₆ alkyl is optionally substituted by one, two or three substituents each independently selected from —C(O)NR^{a}R^{b}, —NR^{a}R^{b}, hydroxyl, S(O)_{W}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;

In certain embodiments, R¹ is H.

In some embodiments, X is NR².

In embodiments, R² is —C(O)( C₁—C₆alkyl)X′, wherein X′ is a halogen.

In some embodiments, R² is —C(O)(C₁—C₆alkyl)X′, wherein X′ is —O—P(O)(R⁴¹R⁴²), wherein R⁴¹ is selected from —O(C₁—C₆alkyl) and —O—phenyl, and R⁴² is —NH(C₁—C₆alkyl) optionally substituted by —C(O)—O(C₁—C₆alkyl).

In embodiments, R³ is C₁—C₂alkyl, optionally substituted by one or two substituents each independently selected from phenyl and halogen; and phenyl, independently for each occurrence, is optionally substituted by one, two or three substituents each independently selected from hydroxyl, halogen, —C(O)—C₁—C₃alkyl, methyl, and CF₃.

In some embodiments, R³ is —CH₂—phenyl, wherein phenyl is optionally substituted by one, two or three substituents each independently selected from hydroxyl, halogen, —C(O)—C₁—C₃alkyl, methyl, and CF₃.

In certain embodiments, the compound of Formula A is selected from the group consisting of:

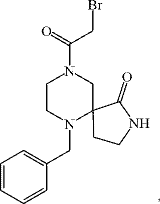

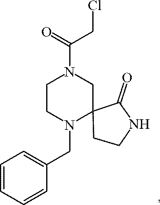

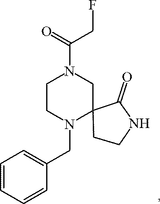

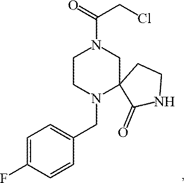

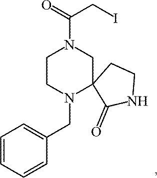

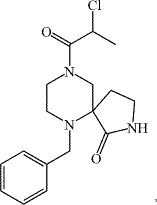

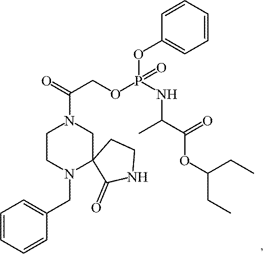

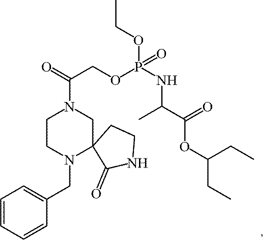

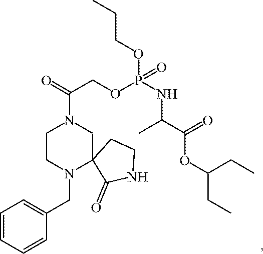

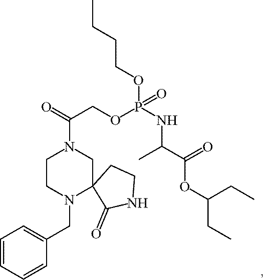

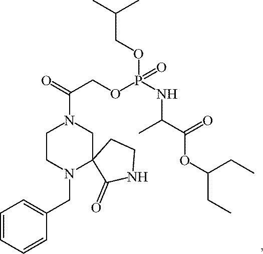

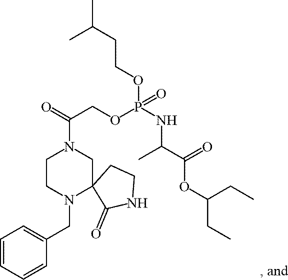

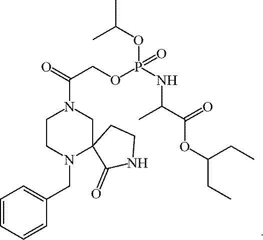

In certain embodiments, R¹ can be —C(O)—O—C₁—C₆ alkyl. For example, R¹ can be tert-butyloxycarbonyl.

In certain embodiments, R¹ can be C₁—C₆alkyl, optionally substituted by benzyl or one, two or three fluorines. For example, R¹ can be methyl; while in some embodiments, R¹ can be

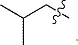

In certain embodiments, R¹ can be —C(O)—C₁—C₆alkyl, where —C(O)—C₁—C₆alkyl can be represented by:

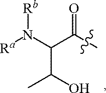

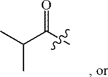

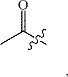

wherein R^{a} and R^{b} can be independently selected for each occurrence from the group consisting of hydrogen and —C₁—C₆alkyl.

In some embodiments, R¹ can be benzyl.

In certain embodiments, X can be O; while in certain embodiments, X can be NR².

In certain embodiments, R² can be H.

In certain embodiments, R² can be C₁—C₆alkyl, optionally substituted by benzyl or one, two or three fluorines, —C(O)—C₁—C₆alkyl, or —C(O)—O—C₁—C₆ alkyl. For example, R² can be methyl or

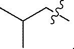

In some embodiments, R² can be benzyl.

In certain embodiments, R² can be —C(O)—C₁—C₆alkyl, where —C(O)—C₁—C₆alkyl can be represented by:

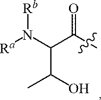

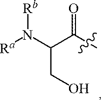

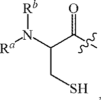

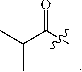

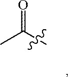

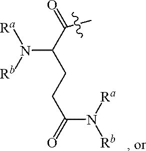

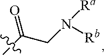

wherein R^{a} and R^{b} can be each independently selected for each occurrence from the group consisting of hydrogen and —C₁—C₆alkyl.

In some embodiments, R² can be —C(O)—O—C₁—C₆ alkyl, for example, tert-butyloxycarbonyl.

In certain embodiments, p is 2.

In some embodiments, R³ can be H.

In certain embodiments, R³ can be selected from the group consisting of:

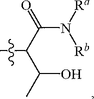

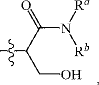

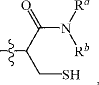

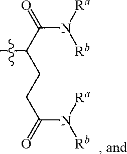

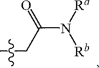

wherein R^{a} and R^{b} are each independently selected for each occurrence from the group consisting of hydrogen and —C₁—C₆alkyl.

In some embodiments, the compound is selected from the compounds delineated in the chart below, and includes pharmaceutically acceptable salts and/or stereoisomers thereof. In certain embodiments, a compound having Formula (A) includes a compound having the formula:

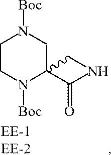

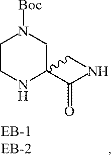

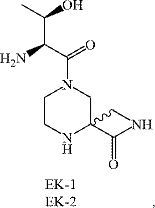

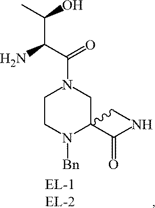

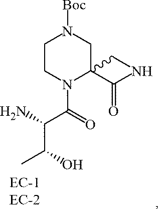

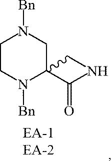

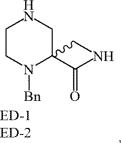

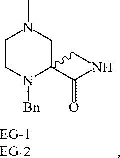

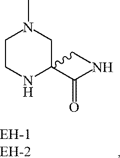

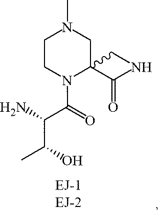

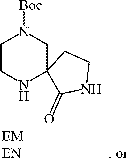

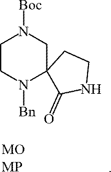

In certain embodiments, for compound of Formula (A), Formula (A) is:

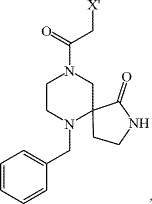

wherein X′ is Br, Cl, or F.

In certain embodiments, for compound of Formula (A), Formula (A) is:

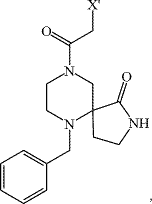

wherein X′ is I or —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl).

In some embodiments, for compound of Formula (A), X′ is selected from the group consisting of

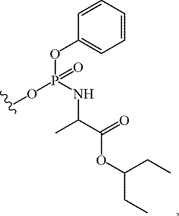

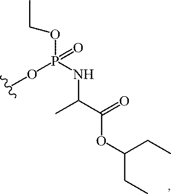

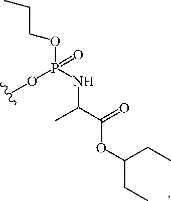

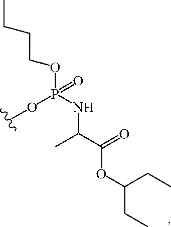

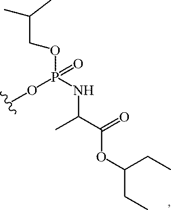

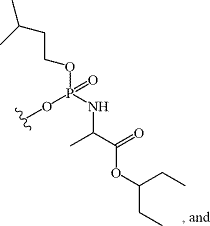

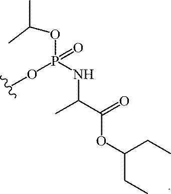

In some embodiments, the compound of Formula (A) is a compound having Formula (A-I):

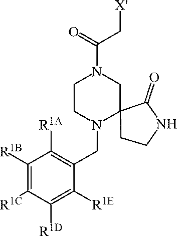

wherein:
X′ is a halogen; and
one, two or three of R^{1A}, R^{1B}, R^{1C}, R^{1D}, and R^{1E} are optionally each independently selected from the group consisting of H, hydroxyl, halogen, —C(O)—O—C₁—C₃alkyl, —C(O)—C₁—C₃alkyl, methyl, and CF₃.

In some embodiments, the compound of Formula (A-I) is selected from the group consisting of a compound having Formula (A-II); a compound having Formula (A-III); and a compound having Formula (A-IV), wherein:
the compound having Formula (A-II) is:

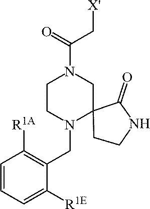

wherein:
   X′ is a halogen; and
   R^{1A} and R^{1E} are optionally each independently selected from the group consisting of hydroxyl, halogen, —C(O)—O—C₁—C₃alkyl, —C(O)—C₁—C₃alkyl, methyl, and CF₃;
the compound having Formula (A-III) is:

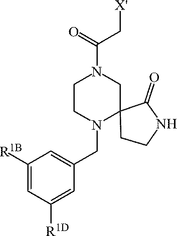

wherein:
X′ is a halogen; and
R^{1B} and R^{1D} are optionally each independently selected from the group consisting of hydroxyl, halogen, —C(O)—O—C₁—C₃alkyl, —C(O)—C₁—C₃alkyl, methyl, and CF₃; and the compound having Formula (A-IV) is:

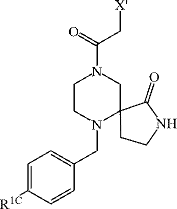

wherein:
X′ is a halogen; and
R^{1C} is optionally selected from the group consisting of hydroxyl, halogen, —C(O)—O—C₁—C₃alkyl, —C(O)—C₁—C₃alkyl, methyl, and CF₃.

In some embodiments, the compound of Formula (A-I) is selected from the group consisting of a compound having Formula (A-V); and a compound having Formula (A-VI), wherein:
the compound having Formula (A-V) is:

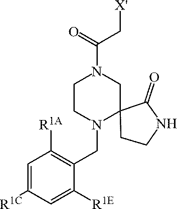

wherein:
   X′ is a halogen; and
   R^{1A}, R^{1C}, and R^{1E} are optionally each independently selected from the group consisting of hydroxyl, halogen, —C(O)—O—C₁—C₃alkyl, —C(O)—C₁—C₃alkyl, methyl, and CF₃; and the compound having Formula (A-VI) is: 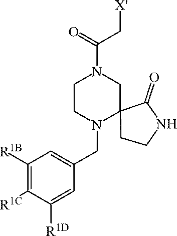
   wherein:
   X′ is a halogen; and
   R^{1B}, R^{1C}, and R^{1D} are optionally each independently selected from the group consisting of hydroxyl, halogen, —C(O)—O—C₁—C₃alkyl, —C(O)—C₁—C₃alkyl, methyl, and CF₃.

In some embodiments of the methods of the invention described herein, the compound has the Formula (A-Ia):

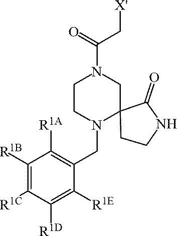

wherein:
X′ is a halogen; and
one, two, three or four of R^{1A}, R^{1B}, R^{1C}, R^{1D}, and R^{1E} are optionally each independently selected from the group consisting of hydroxyl, halogen, —C—O—C₁—C₃alkyl, —C(O)—O—C₁—C₃alkyl, —C(O)—C₁—C₃alkyl, methyl, and CF₃.

In some embodiments, the compound of Formula (A-Ia) is selected from the group consisting of a compound having Formula (A-IIa); a compound having Formula (A-IIIa); and a compound having Formula (A-IVa), wherein:
the compound having Formula (A-IIa) is:

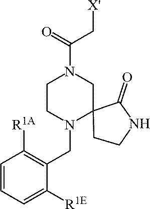

wherein:
   X′ is a halogen; and
   R^{1A} and R^{1E} are optionally each independently selected from the group consisting of hydroxyl, halogen, —C—O—C₁—C₃alkyl, —C(O)—O—C₁—C₃alkyl, —C(O)—C₁—C₃alkyl, methyl, and CF₃;
the compound having Formula (A-IIIa) is:

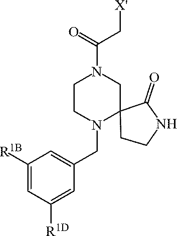

wherein:
   X′ is a halogen; and
   R^{1B} and R^{1D} are optionally each independently selected from the group consisting of hydroxyl, halogen, —C—O—C₁—C₃alkyl, —C(O)—O—C₁—C₃alkyl, —C(O)—C₁—C₃alkyl, methyl,
   and CF₃; and
the compound having Formula (A-IVa) is:

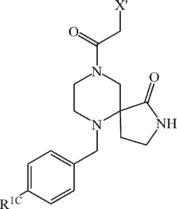

wherein:
   X′ is a halogen; and
   R^{1C} is optionally selected from the group consisting of hydroxyl, halogen, —C—O—C₁—C₃alkyl, —C(O)—O—C₁—C₃alkyl, —C(O)—C₁—C₃alkyl, methyl, and CF₃.

In some embodiments, the compound of Formula (A-Ia) is selected from the group consisting of a compound having Formula (A-Va); and a compound having Formula (A-VIa), wherein:
the compound having Formula (A-Va) is:

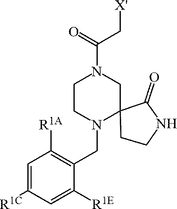

wherein:
   X′ is a halogen; and
   R^{1A}, R^{1C}, and R^{1E} are optionally each independently selected from the group consisting of hydroxyl, halogen, —C—O—C₁—C₃alkyl, —C(O)—O—C₁—C₃alkyl, —C(O)—C₁—C₃alkyl, methyl, and CF₃; and
the compound having Formula (A-VIa) is:

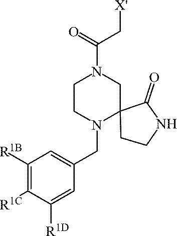

wherein:
   X′ is a halogen; and
   R^{1B}, R^{1C}, and R^{1D} are optionally each independently selected from the group consisting of hydroxyl, halogen, —C—O—C₁—C₃alkyl, —C(O)—O—C₁—C₃alkyl, —C(O)—C₁—C₃alkyl, methyl, and CF₃.

In some embodiments, the compound of Formula (A-Ia) is a compound having Formula (A-VIIa), wherein:
the compound having Formula (A-VIIa) is:

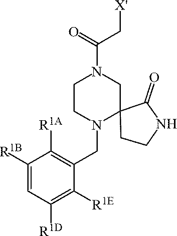

wherein:
   X′ is a halogen; and
   R^{1A}, R^{1B}, R^{1D}, and R^{1E} are optionally each independently selected from the group consisting of hydroxyl, halogen, —C—O—C₁—C₃alkyl, —C(O)—O—C₁—C₃alkyl, —C(O)—C₁—C₃alkyl, methyl, and CF₃.

In some embodiments, the compound of Formula (A) is a compound having Formula (A-I):

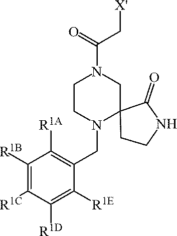

wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH2, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
one, two or three of R^{1A}, R^{1B}, R^{1C}, R^{1D}, and R^{1E} are optionally each independently selected from the group consisting of H, hydroxyl, halogen, —C(O)—O—C₁—C₃alkyl, —C(O)—C₁—C₃alkyl, methyl, and CF₃.

In some embodiments, for Formula (A-I), the compound is selected from the group consisting of a compound having Formula (A-II); a compound having Formula (A-III); and a compound having Formula (A-IV), wherein:
Formula (A-II) is:

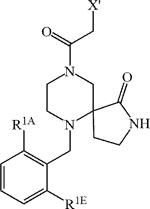

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
   R^{1A} and R^{1E} are optionally each independently selected from the group consisting of hydroxyl, halogen, —C(O)—O—C₁—C₃alkyl, —C(O)—C₁—C₃alkyl, methyl, and CF₃;
Formula (A-III) is:

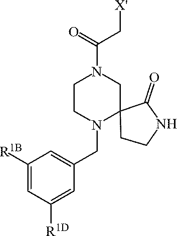

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃-C₆cycloalkyl, C₁—C₆alkoxy, —C(O)—(C₁—C₆ alkyl), and —C(O)—O(C₁—C₆alkyl); and
   R^{1B} and R^{1D} are optionally each independently selected from the group consisting of hydroxyl, halogen, —C(O)—O—C₁—C₃alkyl, —C(O)—C₁—C₃alkyl, methyl, and CF₃; and
Formula (A-IV) is:

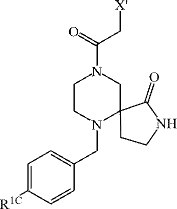

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁-C₆alkyl, —C₃—C₆cycloalkyl, phenyl and naphthyl; and R⁴² is selected from the group consisting of —NH2, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
   R^{1C} is optionally selected from the group consisting of hydroxyl, halogen, —C(O)—O—C₁—C₃alkyl, —C(O)—C₁—C₃alkyl, methyl, and CF₃.

In some embodiments, for Formula (A-I), for Formula (A-I), the compound is selected from the group consisting of a compound having Formula (A-V); and a compound having Formula (A-VI), wherein:
Formula (A-V) is:

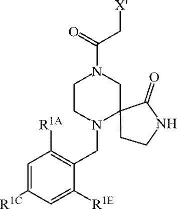

wherein:
   X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
   R^{1A}, R^{1C}, and R^{1E} are optionally each independently selected from the group consisting of hydroxyl, halogen, —C(O)—O—C₁—C₃alkyl, —C(O)—C₁—C₃alkyl, methyl, and CF₃; and Formula (A-VI) is: 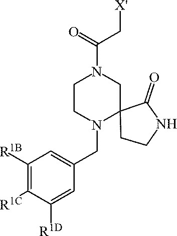
   wherein:
      X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
      R^{1B}, R^{1C}, and R^{1D} are optionally each independently selected from the group consisting of hydroxyl, halogen, —C(O)—O—C₁—C₃alkyl, —C(O)—C₁—C₃alkyl, methyl, and CF₃.

In some embodiments, the compound of Formula (A) is a selected from the group consisting of:

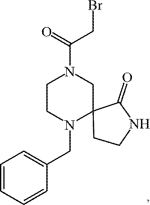

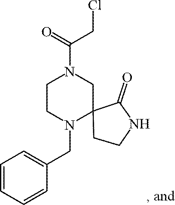

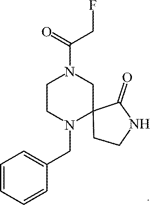

In some embodiments, the compound of Formula (A) is a selected from the group consisting of:

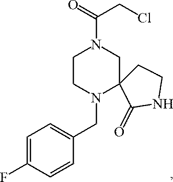

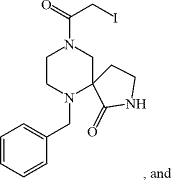

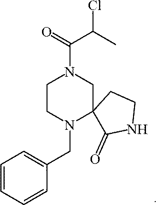

In some embodiments, the compound of Formula (A) is a selected from the group consisting of:

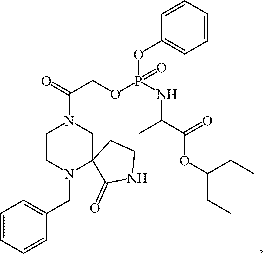

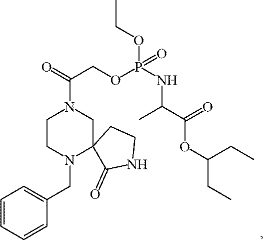

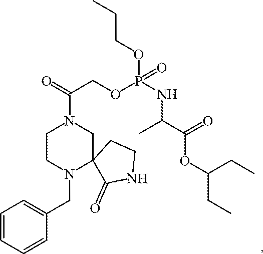

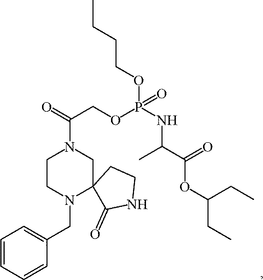

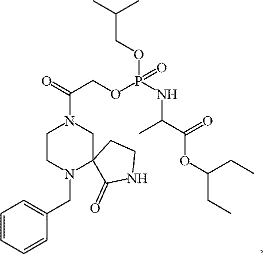

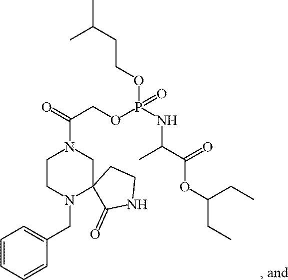

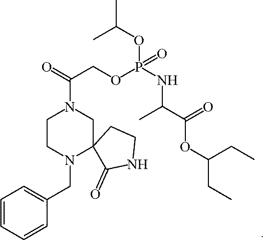

The following are exemplary compounds of Formula (A). It should be appreciated that the compound in the first column is a different stereoisomer, for example, a different enantiomer and/or different diastereomer, from the compound in the second column. In certain examples, the compound in one column may be a mixture of isomers, for example, as described herein.

| | | | |
|---|---|---|---|
| Structure | Compound | Structure | Compound |
| 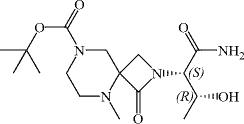 | ER-101 | 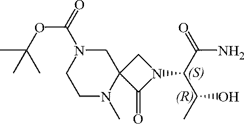 | ER-102 |
| 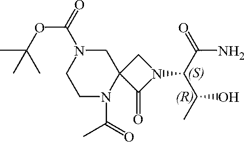 | ER-103 | 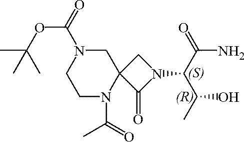 | ER-104 |
| 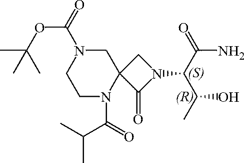 | ER-105 | 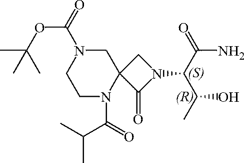 | ER-106 |
| 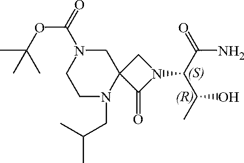 | ER-107 | 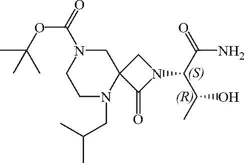 | ER-108 |
| 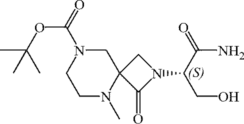 | ER-109 | 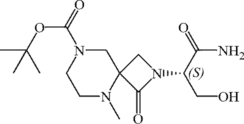 | ER-110 |
| 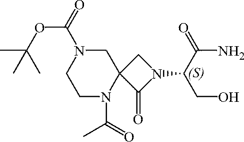 | ER-111 | 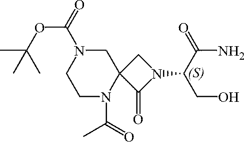 | ER-112 |
| 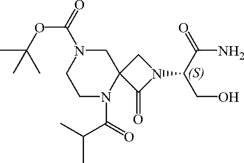 | ER-113 | 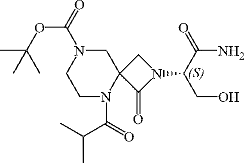 | ER-114 |
| 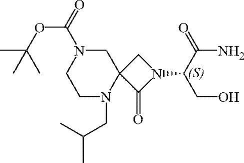 | ER-115 | 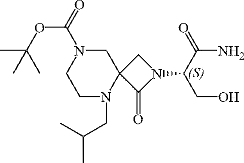 | ER-116 |
| 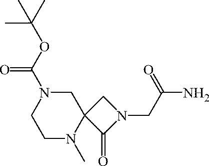 [SMILES: CN1CCN(C(=O)OC(C)(C)C)CC12CN(CC(N)=O)C2=O] | ER-117 | 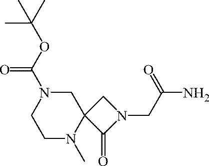 [SMILES: CN1CCN(C(=O)OC(C)(C)C)CC12CN(CC(N)=O)C2=O] | ER-118 |
| 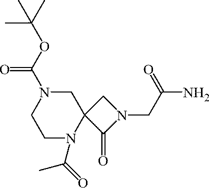 [SMILES: CC(=O)N1CCN(C(=O)OC(C)(C)C)CC12CN(CC(N)=O)C2=O] | ER-119 | 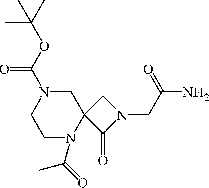 [SMILES: CC(=O)N1CCN(C(=O)OC(C)(C)C)CC12CN(CC(N)=O)C2=O] | ER-120 |
| 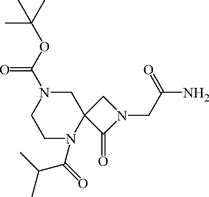 [SMILES: CC(C)C(=O)N1CCN(C(=O)OC(C)(C)C)CC12CN(CC(N)=O)C2=O] | ER-121 | 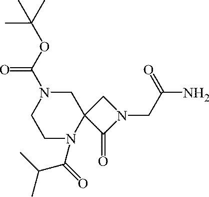 [SMILES: CC(C)C(=O)N1CCN(C(=O)OC(C)(C)C)CC12CN(CC(N)=O)C2=O] | ER-122 |
| 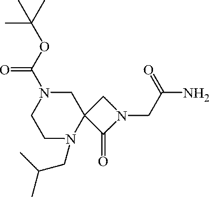 [SMILES: CC(C)CN1CCN(C(=O)OC(C)(C)C)CC12CN(CC(N)=O)C2=O] | ER-123 | 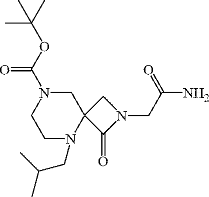 [SMILES: CC(C)CN1CCN(C(=O)OC(C)(C)C)CC12CN(CC(N)=O)C2=O] | ER-124 |
| 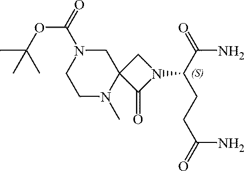 [SMILES: CN1CCN(C(=O)OC(C)(C)C)CC12CN([C@@H](CCC(N)=O)C(N)=O)C2=O] | ER-125 | 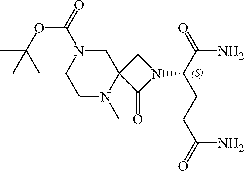 [SMILES: CN1CCN(C(=O)OC(C)(C)C)CC12CN([C@@H](CCC(N)=O)C(N)=O)C2=O] | ER-126 |
| 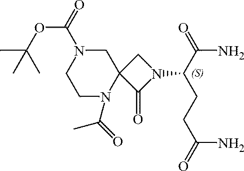 [SMILES: CC(=O)N1CCN(C(=O)OC(C)(C)C)CC12CN([C@@H](CCC(N)=O)C(N)=O)C2=O] | ER-127 | 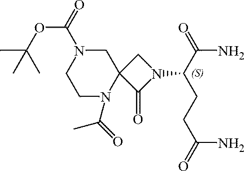 [SMILES: CC(=O)N1CCN(C(=O)OC(C)(C)C)CC12CN([C@@H](CCC(N)=O)C(N)=O)C2=O] | ER-128 |
| 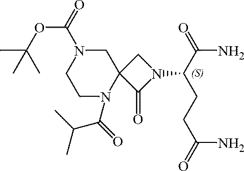 [SMILES: CC(C)C(=O)N1CCN(C(=O)OC(C)(C)C)CC12CN([C@@H](CCC(N)=O)C(N)=O)C2=O] | ER-129 | 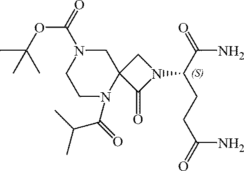 [SMILES: CC(C)C(=O)N1CCN(C(=O)OC(C)(C)C)CC12CN([C@@H](CCC(N)=O)C(N)=O)C2=O] | ER-130 |
| 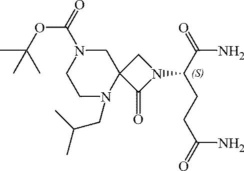 [SMILES: CC(C)CN1CCN(C(=O)OC(C)(C)C)CC12CN([C@@H](CCC(N)=O)C(N)=O)C2=O] | ER-131 | 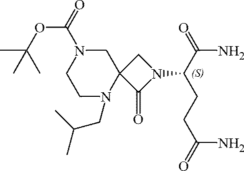 [SMILES: CC(C)CN1CCN(C(=O)OC(C)(C)C)CC12CN([C@@H](CCC(N)=O)C(N)=O)C2=O] | ER-132 |
| 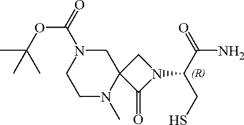 [SMILES: CN1CCN(C(=O)OC(C)(C)C)CC12CN([C@@H](CS)C(N)=O)C2=O] | ER-133 | 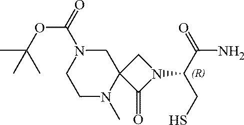 [SMILES: CN1CCN(C(=O)OC(C)(C)C)CC12CN([C@@H](CS)C(N)=O)C2=O] | ER-134 |
| 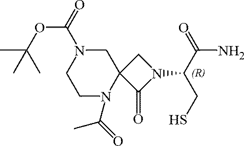 [SMILES: CC(=O)N1CCN(C(=O)OC(C)(C)C)CC12CN([C@@H](CS)C(N)=O)C2=O] | ER-135 | 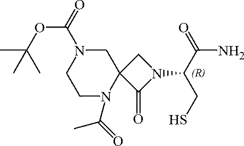 [SMILES: CC(=O)N1CCN(C(=O)OC(C)(C)C)CC12CN([C@@H](CS)C(N)=O)C2=O] | ER-136 |
| 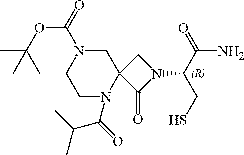 [SMILES: CC(C)C(=O)N1CCN(C(=O)OC(C)(C)C)CC12CN([C@@H](CS)C(N)=O)C2=O] | ER-137 | 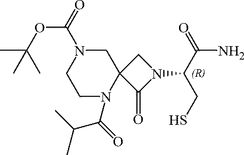 [SMILES: CC(C)C(=O)N1CCN(C(=O)OC(C)(C)C)CC12CN([C@@H](CS)C(N)=O)C2=O] | ER-138 |
| 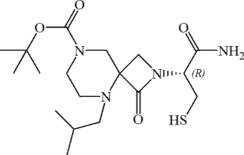 [SMILES: CC(C)CN1CCN(C(=O)OC(C)(C)C)CC12CN([C@@H](CS)C(N)=O)C2=O] | ER-139 | 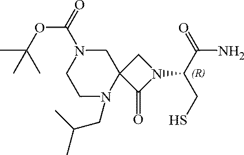 [SMILES: CC(C)CN1CCN(C(=O)OC(C)(C)C)CC12CN([C@@H](CS)C(N)=O)C2=O] | ER-140 |
| 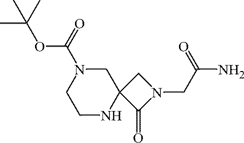 [SMILES: CC(C)(C)OC(=O)N1CCNC2(C1)CN(CC(N)=O)C2=O] | ER-141 | 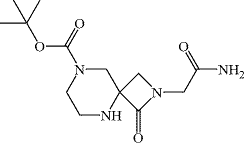 [SMILES: CC(C)(C)OC(=O)N1CCNC2(C1)CN(CC(N)=O)C2=O] | ER-142 |
| 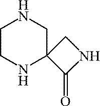 | ER-143 | 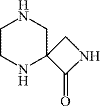 | ER-144 |
| 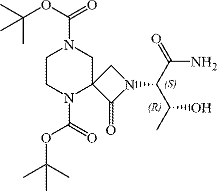 [SMILES: C[C@@H](O)[C@@H](C(N)=O)N1CC2(CN(C(=O)OC(C)(C)C)CCN2C(=O)OC(C)(C)C)C1=O] | ER-145 | 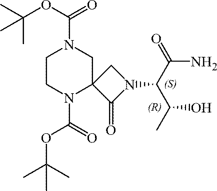 [SMILES: C[C@@H](O)[C@@H](C(N)=O)N1CC2(CN(C(=O)OC(C)(C)C)CCN2C(=O)OC(C)(C)C)C1=O] | ER-146 |
| 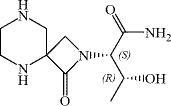 [SMILES: C[C@@H](O)[C@@H](C(N)=O)N1CC2(CNCCN2)C1=O] | ER-147 | 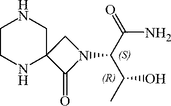 [SMILES: C[C@@H](O)[C@@H](C(N)=O)N1CC2(CNCCN2)C1=O] | ER-148 |
| 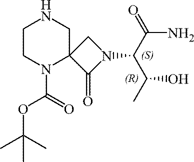 [SMILES: C[C@@H](O)[C@@H](C(N)=O)N1CC2(CNCCN2C(=O)OC(C)(C)C)C1=O] | ER-149 | 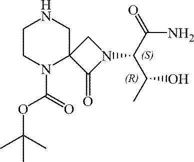 [SMILES: C[C@@H](O)[C@@H](C(N)=O)N1CC2(CNCCN2C(=O)OC(C)(C)C)C1=O] | ER-150 |
| 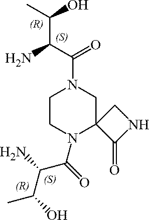 [SMILES: C[C@@H](O)[C@H](N)C(=O)N1CCN(C(=O)[C@@H](N)[C@@H](C)O)C2(CNC2=O)C1] | ER-151 | 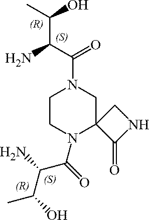 [SMILES: C[C@@H](O)[C@H](N)C(=O)N1CCN(C(=O)[C@@H](N)[C@@H](C)O)C2(CNC2=O)C1] | ER-152 |
| 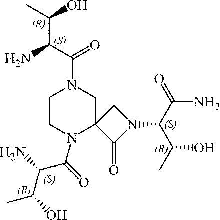 [SMILES: C[C@@H](O)[C@H](N)C(=O)N1CCN(C(=O)[C@@H](N)[C@@H](C)O)C2(C1)CN([C@H](C(N)=O)[C@@H](C)O)C2=O] | ER-153 | 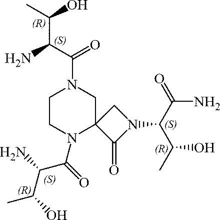 [SMILES: C[C@@H](O)[C@H](N)C(=O)N1CCN(C(=O)[C@@H](N)[C@@H](C)O)C2(C1)CN([C@H](C(N)=O)[C@@H](C)O)C2=O] | ER-154 |
| 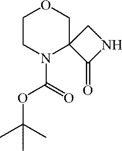 [SMILES: CC(C)(C)OC(=O)N1CCOCC12CNC2=O] | ER-155 | 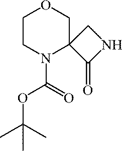 [SMILES: CC(C)(C)OC(=O)N1CCOCC12CNC2=O] | ER-156 |
| 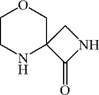 | ER-157 | 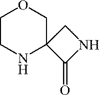 | ER-158 |
| 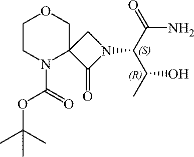 [SMILES: C[C@@H](O)[C@@H](C(N)=O)N1CC2(COCCN2C(=O)OC(C)(C)C)C1=O] | ER-159 | 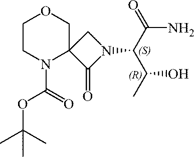 [SMILES: C[C@@H](O)[C@@H](C(N)=O)N1CC2(COCCN2C(=O)OC(C)(C)C)C1=O] | ER-160 |
| 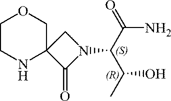 [SMILES: C[C@@H](O)[C@@H](C(N)=O)N1CC2(COCCN2)C1=O] | ER-161 | 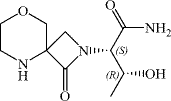 [SMILES: C[C@@H](O)[C@@H](C(N)=O)N1CC2(COCCN2)C1=O] | ER-162 |
| 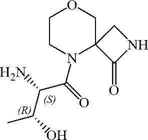 [SMILES: C[C@@H](O)[C@H](N)C(=O)N1CCOCC12CNC2=O] | ER-163 | 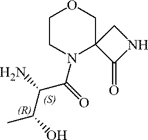 [SMILES: C[C@@H](O)[C@H](N)C(=O)N1CCOCC12CNC2=O] | ER-164 |
| 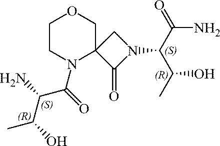 [SMILES: C[C@@H](O)[C@H](N)C(=O)N1CCOCC12CN([C@H](C(N)=O)[C@@H](C)O)C2=O] | ER-165 | 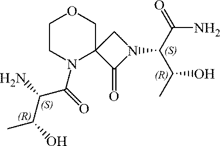 [SMILES: C[C@@H](O)[C@H](N)C(=O)N1CCOCC12CN([C@H](C(N)=O)[C@@H](C)O)C2=O] | ER-166 |
| 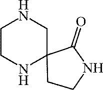 [SMILES: O=C1NCCC12CNCCN2] | ES-301 | 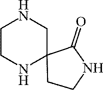 [SMILES: O=C1NCCC12CNCCN2] | ES-302 |
| 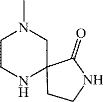 | ES-303 | 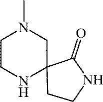 | ES-304 |
| 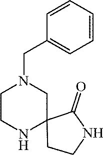 [SMILES: O=C1NCCC12CN(Cc1ccccc1)CCN2] | ES-305 | 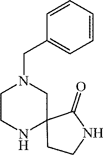 [SMILES: O=C1NCCC12CN(Cc1ccccc1)CCN2] | ES-306 |
| 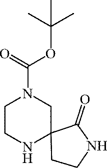 [SMILES: CC(C)(C)OC(=O)N1CCNC2(CCNC2=O)C1] | EM | 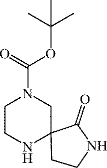 [SMILES: CC(C)(C)OC(=O)N1CCNC2(CCNC2=O)C1] | EN |
| 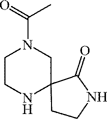 | ES-307 | 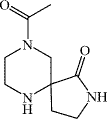 | ES-308 |
| 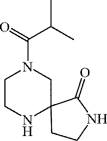 [SMILES: CC(C)C(=O)N1CCNC2(CCNC2=O)C1] | ES-309 | 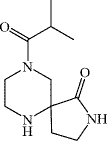 [SMILES: CC(C)C(=O)N1CCNC2(CCNC2=O)C1] | ES-310 |
| 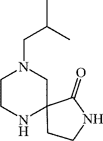 | ES-311 | 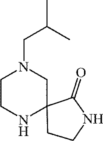 | ES-312 |
| 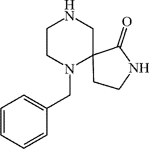 [SMILES: O=C1NCCC12CNCCN2Cc1ccccc1] | ES-313 | 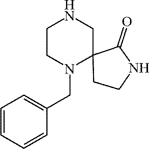 [SMILES: O=C1NCCC12CNCCN2Cc1ccccc1] | ES-314 |
| 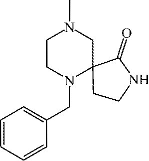 [SMILES: CN1CCN(Cc2ccccc2)C2(CCNC2=O)C1] | ES-315 | 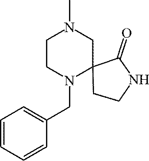 [SMILES: CN1CCN(Cc2ccccc2)C2(CCNC2=O)C1] | ES-316 |
| 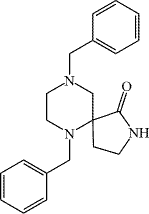 [SMILES: O=C1NCCC12CN(Cc1ccccc1)CCN2Cc1ccccc1] | ES-317 | 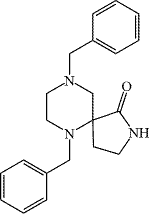 [SMILES: O=C1NCCC12CN(Cc1ccccc1)CCN2Cc1ccccc1] | ES-318 |
| 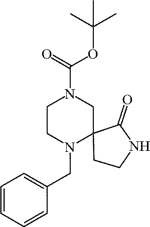 [SMILES: CC(C)(C)OC(=O)N1CCN(Cc2ccccc2)C2(CCNC2=O)C1] | MO | 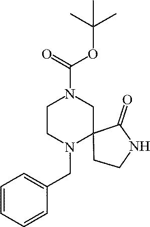 [SMILES: CC(C)(C)OC(=O)N1CCN(Cc2ccccc2)C2(CCNC2=O)C1] | MP |
| 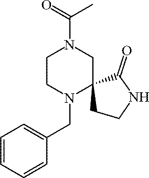 [SMILES: CC(=O)N1CCN(Cc2ccccc2)[C@]2(CCNC2=O)C1] | ES-319 | 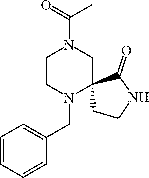 [SMILES: CC(=O)N1CCN(Cc2ccccc2)[C@@]2(CCNC2=O)C1] | ES-320 |
| 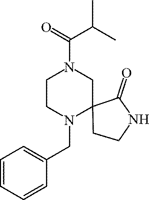 [SMILES: CC(C)C(=O)N1CCN(Cc2ccccc2)C2(CCNC2=O)C1] | ES-321 | 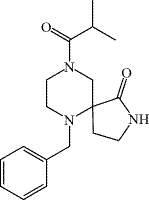 [SMILES: CC(C)C(=O)N1CCN(Cc2ccccc2)C2(CCNC2=O)C1] | ES-322 |
| 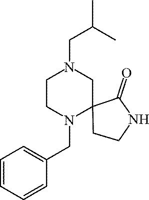 [SMILES: CC(C)CN1CCN(Cc2ccccc2)C2(CCNC2=O)C1] | ES-323 | 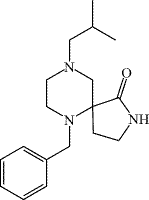 [SMILES: CC(C)CN1CCN(Cc2ccccc2)C2(CCNC2=O)C1] | ES-324 |
| 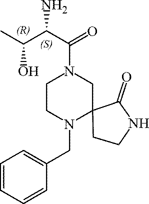 [SMILES: C[C@@H](O)[C@H](N)C(=O)N1CCN(Cc2ccccc2)C2(CCNC2=O)C1] | ES-325 | 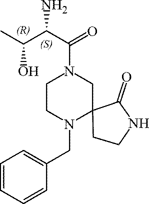 [SMILES: C[C@@H](O)[C@H](N)C(=O)N1CCN(Cc2ccccc2)C2(CCNC2=O)C1] | ES-326 |
| 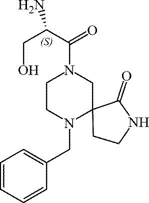 [SMILES: N[C@@H](CO)C(=O)N1CCN(Cc2ccccc2)C2(CCNC2=O)C1] | ES-327 | 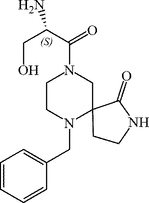 [SMILES: N[C@@H](CO)C(=O)N1CCN(Cc2ccccc2)C2(CCNC2=O)C1] | ES-328 |
| 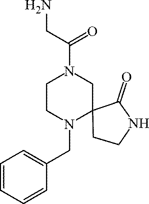 [SMILES: NCC(=O)N1CCN(Cc2ccccc2)C2(CCNC2=O)C1] | ES-329 | 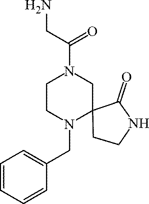 [SMILES: NCC(=O)N1CCN(Cc2ccccc2)C2(CCNC2=O)C1] | ES-330 |
| 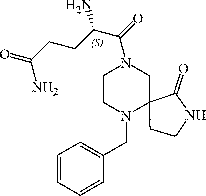 [SMILES: NC(=O)CC[C@H](N)C(=O)N1CCN(Cc2ccccc2)C2(CCNC2=O)C1] | ES-331 | 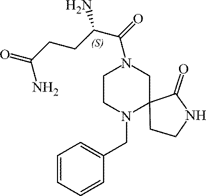 [SMILES: NC(=O)CC[C@H](N)C(=O)N1CCN(Cc2ccccc2)C2(CCNC2=O)C1] | ES-332 |
| 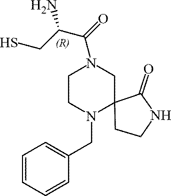 [SMILES: N[C@@H](CS)C(=O)N1CCN(Cc2ccccc2)C2(CCNC2=O)C1] | ES-333 | 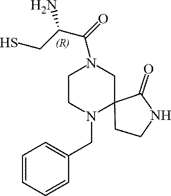 [SMILES: N[C@@H](CS)C(=O)N1CCN(Cc2ccccc2)C2(CCNC2=O)C1] | ES-334 |
| 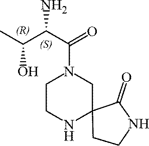 [SMILES: C[C@@H](O)[C@H](N)C(=O)N1CCNC2(CCNC2=O)C1] | ES-335 | 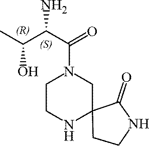 [SMILES: C[C@@H](O)[C@H](N)C(=O)N1CCNC2(CCNC2=O)C1] | ES-336 |
| 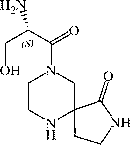 [SMILES: N[C@@H](CO)C(=O)N1CCNC2(CCNC2=O)C1] | ES-337 | 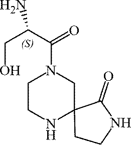 [SMILES: N[C@@H](CO)C(=O)N1CCNC2(CCNC2=O)C1] | ES-338 |
| 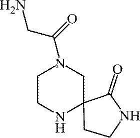 [SMILES: NCC(=O)N1CCNC2(CCNC2=O)C1] | ES-339 | 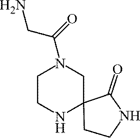 [SMILES: NCC(=O)N1CCNC2(CCNC2=O)C1] | ES-340 |
| 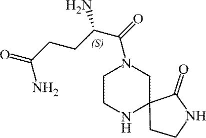 [SMILES: NC(=O)CC[C@H](N)C(=O)N1CCNC2(CCNC2=O)C1] | ES-341 | 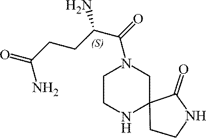 [SMILES: NC(=O)CC[C@H](N)C(=O)N1CCNC2(CCNC2=O)C1] | ES-342 |
| 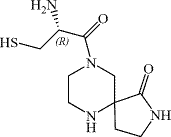 [SMILES: N[C@@H](CS)C(=O)N1CCNC2(CCNC2=O)C1] | ES-343 | 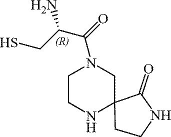 [SMILES: N[C@@H](CS)C(=O)N1CCNC2(CCNC2=O)C1] | ES-344 |
| 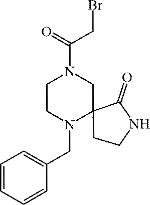 [SMILES: O=C(CBr)N1CCN(Cc2ccccc2)C2(CCNC2=O)C1] | ET-101 | 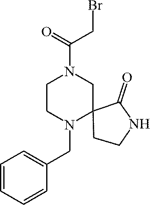 [SMILES: O=C(CBr)N1CCN(Cc2ccccc2)C2(CCNC2=O)C1] | ET-102 |
| 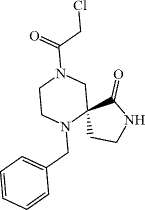 [SMILES: O=C(CCl)N1CCN(Cc2ccccc2)[C@@]2(CCNC2=O)C1] | ET-103 | 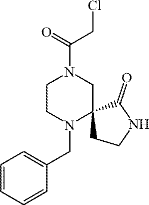 [SMILES: O=C(CCl)N1CCN(Cc2ccccc2)[C@]2(CCNC2=O)C1] | ET-104 |
| 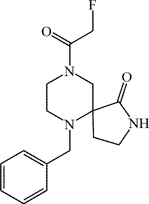 [SMILES: O=C(CF)N1CCN(Cc2ccccc2)C2(CCNC2=O)C1] | ET-105 | 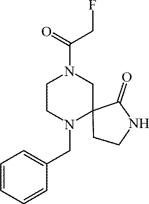 [SMILES: O=C(CF)N1CCN(Cc2ccccc2)C2(CCNC2=O)C1] | ET-106 |
| 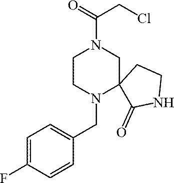 [SMILES: O=C(CCl)N1CCN(Cc2ccc(F)cc2)C2(CCNC2=O)C1] | ET-107 | 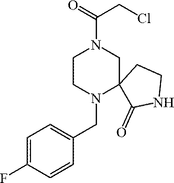 [SMILES: O=C(CCl)N1CCN(Cc2ccc(F)cc2)C2(CCNC2=O)C1] | ET-108 |
| 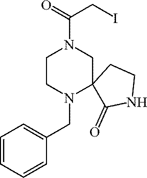 [SMILES: O=C(CI)N1CCN(Cc2ccccc2)C2(CCNC2=O)C1] | ET-109 | 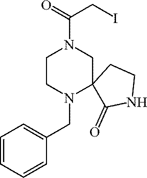 [SMILES: O=C(CI)N1CCN(Cc2ccccc2)C2(CCNC2=O)C1] | ET-110 |
| 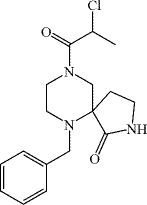 [SMILES: CC(Cl)C(=O)N1CCN(Cc2ccccc2)C2(CCNC2=O)C1] | ET-111 | 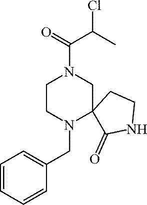 [SMILES: CC(Cl)C(=O)N1CCN(Cc2ccccc2)C2(CCNC2=O)C1] | ET-112 |
| 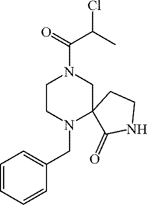 [SMILES: CC(Cl)C(=O)N1CCN(Cc2ccccc2)C2(CCNC2=O)C1] | ET-113 | 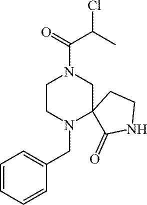 [SMILES: CC(Cl)C(=O)N1CCN(Cc2ccccc2)C2(CCNC2=O)C1] | ET-114 |
| 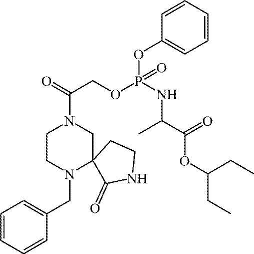 [SMILES: CCC(CC)OC(=O)C(C)NP(=O)(OCC(=O)N1CCN(Cc2ccccc2)C2(CCNC2=O)C1)Oc1ccccc1] | ET-115 | 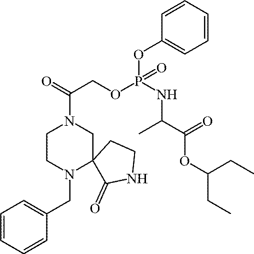 [SMILES: CCC(CC)OC(=O)C(C)NP(=O)(OCC(=O)N1CCN(Cc2ccccc2)C2(CCNC2=O)C1)Oc1ccccc1] | ET-116 |
| 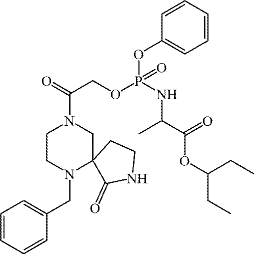 [SMILES: CCC(CC)OC(=O)C(C)NP(=O)(OCC(=O)N1CCN(Cc2ccccc2)C2(CCNC2=O)C1)Oc1ccccc1] | ET-117 | 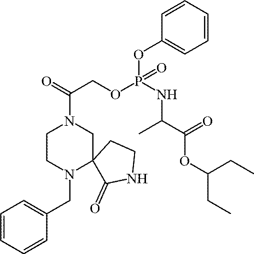 [SMILES: CCC(CC)OC(=O)C(C)NP(=O)(OCC(=O)N1CCN(Cc2ccccc2)C2(CCNC2=O)C1)Oc1ccccc1] | ET-118 |
| 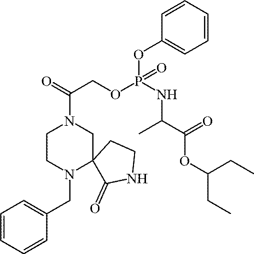 [SMILES: CCC(CC)OC(=O)C(C)NP(=O)(OCC(=O)N1CCN(Cc2ccccc2)C2(CCNC2=O)C1)Oc1ccccc1] | ET-119 | 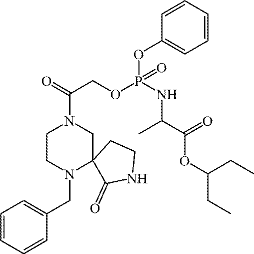 [SMILES: CCC(CC)OC(=O)C(C)NP(=O)(OCC(=O)N1CCN(Cc2ccccc2)C2(CCNC2=O)C1)Oc1ccccc1] | ET-120 |
| 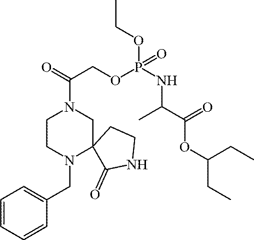 [SMILES: CCOP(=O)(NC(C)C(=O)OC(CC)CC)OCC(=O)N1CCN(Cc2ccccc2)C2(CCNC2=O)C1] | ET-121 | 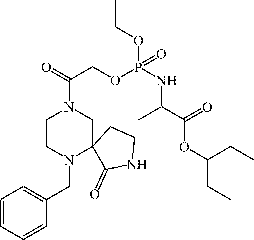 [SMILES: CCOP(=O)(NC(C)C(=O)OC(CC)CC)OCC(=O)N1CCN(Cc2ccccc2)C2(CCNC2=O)C1] | ET-122 |
| 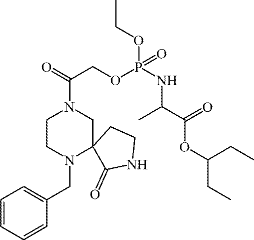 [SMILES: CCOP(=O)(NC(C)C(=O)OC(CC)CC)OCC(=O)N1CCN(Cc2ccccc2)C2(CCNC2=O)C1] | ET-123 | 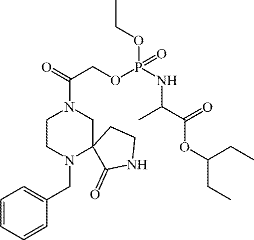 [SMILES: CCOP(=O)(NC(C)C(=O)OC(CC)CC)OCC(=O)N1CCN(Cc2ccccc2)C2(CCNC2=O)C1] | ET-124 |
| 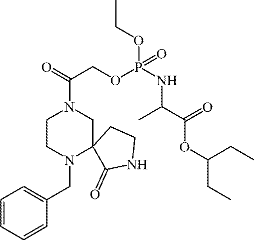 [SMILES: CCOP(=O)(NC(C)C(=O)OC(CC)CC)OCC(=O)N1CCN(Cc2ccccc2)C2(CCNC2=O)C1] | ET-125 | 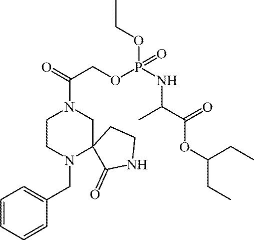 [SMILES: CCOP(=O)(NC(C)C(=O)OC(CC)CC)OCC(=O)N1CCN(Cc2ccccc2)C2(CCNC2=O)C1] | ET-126 |
| 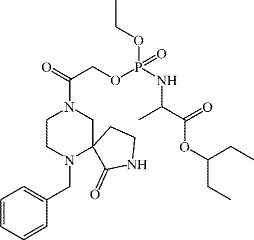 [SMILES: CCOP(=O)(NC(C)C(=O)OC(CC)CC)OCC(=O)N1CCN(Cc2ccccc2)C2(CCNC2=O)C1] | ET-127 | 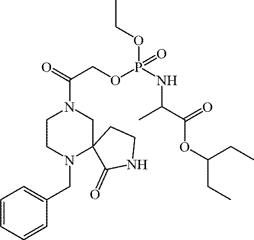 [SMILES: CCOP(=O)(NC(C)C(=O)OC(CC)CC)OCC(=O)N1CCN(Cc2ccccc2)C2(CCNC2=O)C1] | ET-128 |
| 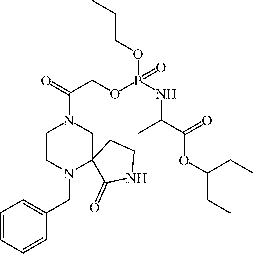 [SMILES: CCCOP(=O)(NC(C)C(=O)OC(CC)CC)OCC(=O)N1CCN(Cc2ccccc2)C2(CCNC2=O)C1] | ET-129 | 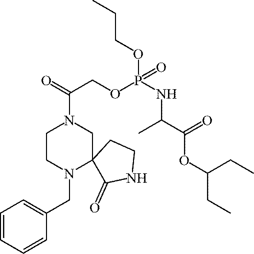 [SMILES: CCCOP(=O)(NC(C)C(=O)OC(CC)CC)OCC(=O)N1CCN(Cc2ccccc2)C2(CCNC2=O)C1] | ET-130 |
| 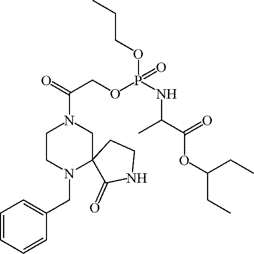 [SMILES: CCCOP(=O)(NC(C)C(=O)OC(CC)CC)OCC(=O)N1CCN(Cc2ccccc2)C2(CCNC2=O)C1] | ET-131 | 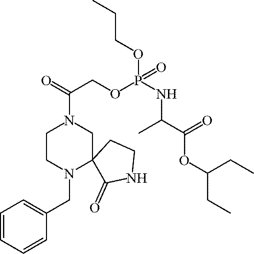 [SMILES: CCCOP(=O)(NC(C)C(=O)OC(CC)CC)OCC(=O)N1CCN(Cc2ccccc2)C2(CCNC2=O)C1] | ET-132 |
| 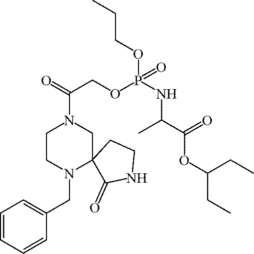 [SMILES: CCCOP(=O)(NC(C)C(=O)OC(CC)CC)OCC(=O)N1CCN(Cc2ccccc2)C2(CCNC2=O)C1] | ET-133 | 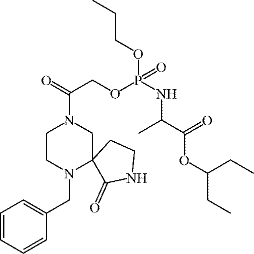 [SMILES: CCCOP(=O)(NC(C)C(=O)OC(CC)CC)OCC(=O)N1CCN(Cc2ccccc2)C2(CCNC2=O)C1] | ET-134 |
| 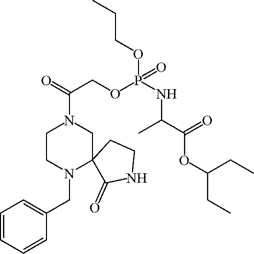 [SMILES: CCCOP(=O)(NC(C)C(=O)OC(CC)CC)OCC(=O)N1CCN(Cc2ccccc2)C2(CCNC2=O)C1] | ET-135 | 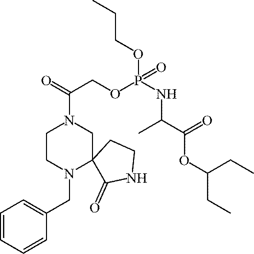 [SMILES: CCCOP(=O)(NC(C)C(=O)OC(CC)CC)OCC(=O)N1CCN(Cc2ccccc2)C2(CCNC2=O)C1] | ET-136 |
| 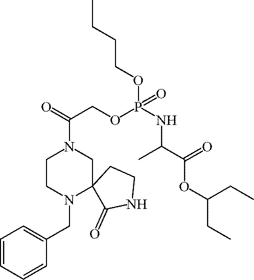 [SMILES: CCCCOP(=O)(NC(C)C(=O)OC(CC)CC)OCC(=O)N1CCN(Cc2ccccc2)C2(CCNC2=O)C1] | ET-137 | 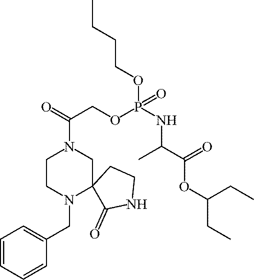 [SMILES: CCCCOP(=O)(NC(C)C(=O)OC(CC)CC)OCC(=O)N1CCN(Cc2ccccc2)C2(CCNC2=O)C1] | ET-138 |
| 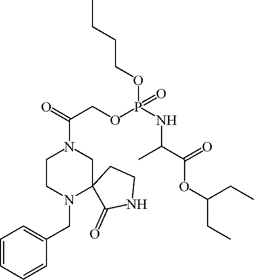 [SMILES: CCCCOP(=O)(NC(C)C(=O)OC(CC)CC)OCC(=O)N1CCN(Cc2ccccc2)C2(CCNC2=O)C1] | ET-139 | 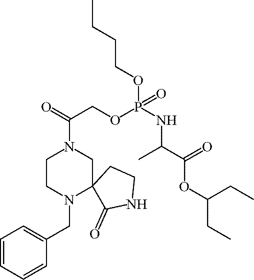 [SMILES: CCCCOP(=O)(NC(C)C(=O)OC(CC)CC)OCC(=O)N1CCN(Cc2ccccc2)C2(CCNC2=O)C1] | ET-140 |
| 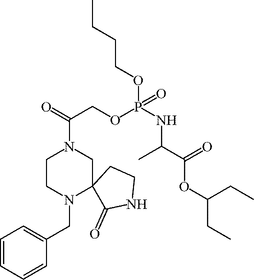 [SMILES: CCCCOP(=O)(NC(C)C(=O)OC(CC)CC)OCC(=O)N1CCN(Cc2ccccc2)C2(CCNC2=O)C1] | ET-141 | 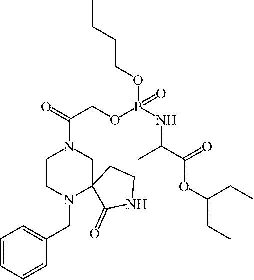 [SMILES: CCCCOP(=O)(NC(C)C(=O)OC(CC)CC)OCC(=O)N1CCN(Cc2ccccc2)C2(CCNC2=O)C1] | ET-142 |
| 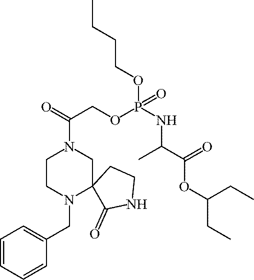 [SMILES: CCCCOP(=O)(NC(C)C(=O)OC(CC)CC)OCC(=O)N1CCN(Cc2ccccc2)C2(CCNC2=O)C1] | ET-143 | 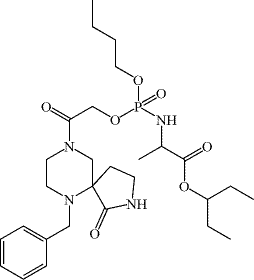 [SMILES: CCCCOP(=O)(NC(C)C(=O)OC(CC)CC)OCC(=O)N1CCN(Cc2ccccc2)C2(CCNC2=O)C1] | ET-144 |
| 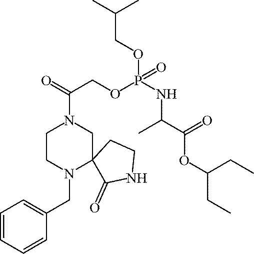 [SMILES: CCC(CC)OC(=O)C(C)NP(=O)(OCC(=O)N1CCN(Cc2ccccc2)C2(CCNC2=O)C1)OCC(C)C] | ET-145 | 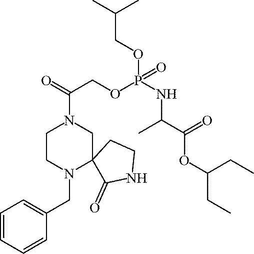 [SMILES: CCC(CC)OC(=O)C(C)NP(=O)(OCC(=O)N1CCN(Cc2ccccc2)C2(CCNC2=O)C1)OCC(C)C] | ET-146 |
| 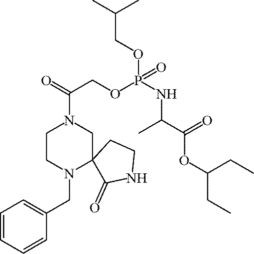 [SMILES: CCC(CC)OC(=O)C(C)NP(=O)(OCC(=O)N1CCN(Cc2ccccc2)C2(CCNC2=O)C1)OCC(C)C] | ET-147 | 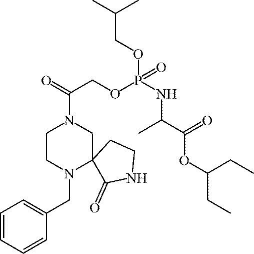 [SMILES: CCC(CC)OC(=O)C(C)NP(=O)(OCC(=O)N1CCN(Cc2ccccc2)C2(CCNC2=O)C1)OCC(C)C] | ET-148 |
| 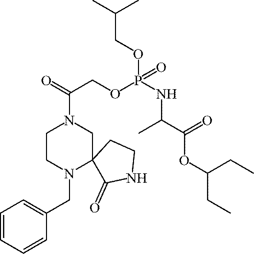 [SMILES: CCC(CC)OC(=O)C(C)NP(=O)(OCC(=O)N1CCN(Cc2ccccc2)C2(CCNC2=O)C1)OCC(C)C] | ET-149 | 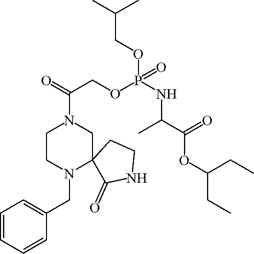 [SMILES: CCC(CC)OC(=O)C(C)NP(=O)(OCC(=O)N1CCN(Cc2ccccc2)C2(CCNC2=O)C1)OCC(C)C] | ET-150 |
| 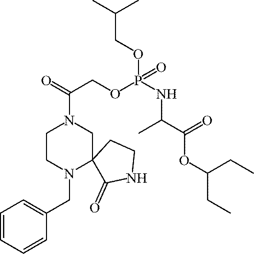 [SMILES: CCC(CC)OC(=O)C(C)NP(=O)(OCC(=O)N1CCN(Cc2ccccc2)C2(CCNC2=O)C1)OCC(C)C] | ET-151 | 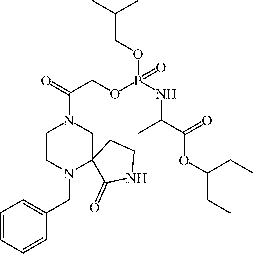 [SMILES: CCC(CC)OC(=O)C(C)NP(=O)(OCC(=O)N1CCN(Cc2ccccc2)C2(CCNC2=O)C1)OCC(C)C] | ET-152 |
| 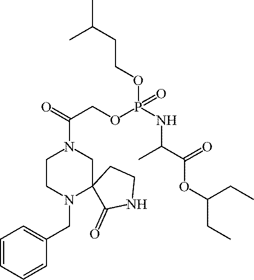 [SMILES: CCC(CC)OC(=O)C(C)NP(=O)(OCCC(C)C)OCC(=O)N1CCN(Cc2ccccc2)C2(CCNC2=O)C1] | ET-153 | 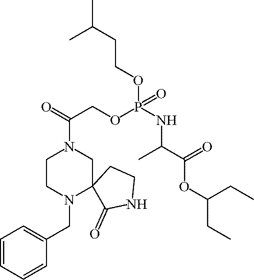 [SMILES: CCC(CC)OC(=O)C(C)NP(=O)(OCCC(C)C)OCC(=O)N1CCN(Cc2ccccc2)C2(CCNC2=O)C1] | ET-154 |
| 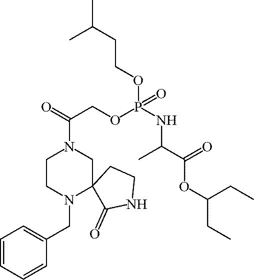 [SMILES: CCC(CC)OC(=O)C(C)NP(=O)(OCCC(C)C)OCC(=O)N1CCN(Cc2ccccc2)C2(CCNC2=O)C1] | ET-155 | 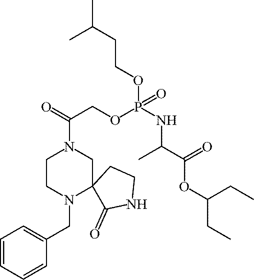 [SMILES: CCC(CC)OC(=O)C(C)NP(=O)(OCCC(C)C)OCC(=O)N1CCN(Cc2ccccc2)C2(CCNC2=O)C1] | ET-156 |
| 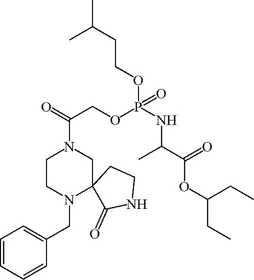 [SMILES: CCC(CC)OC(=O)C(C)NP(=O)(OCCC(C)C)OCC(=O)N1CCN(Cc2ccccc2)C2(CCNC2=O)C1] | ET-157 | 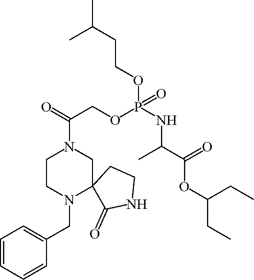 [SMILES: CCC(CC)OC(=O)C(C)NP(=O)(OCCC(C)C)OCC(=O)N1CCN(Cc2ccccc2)C2(CCNC2=O)C1] | ET-158 |
| 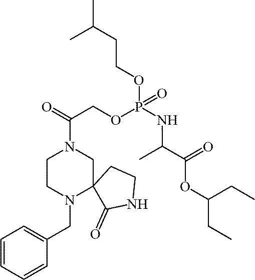 [SMILES: CCC(CC)OC(=O)C(C)NP(=O)(OCCC(C)C)OCC(=O)N1CCN(Cc2ccccc2)C2(CCNC2=O)C1] | ET-160 | 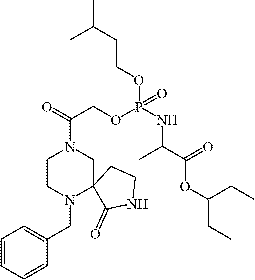 [SMILES: CCC(CC)OC(=O)C(C)NP(=O)(OCCC(C)C)OCC(=O)N1CCN(Cc2ccccc2)C2(CCNC2=O)C1] | ET-161 |
| 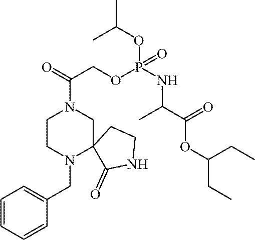 [SMILES: CCC(CC)OC(=O)C(C)NP(=O)(OCC(=O)N1CCN(Cc2ccccc2)C2(CCNC2=O)C1)OC(C)C] | ET-162 | 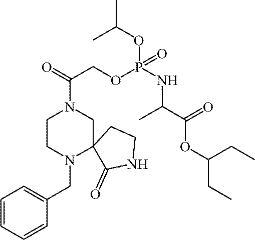 [SMILES: CCC(CC)OC(=O)C(C)NP(=O)(OCC(=O)N1CCN(Cc2ccccc2)C2(CCNC2=O)C1)OC(C)C] | ET-163 |
| 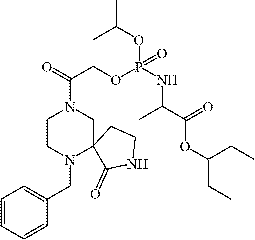 [SMILES: CCC(CC)OC(=O)C(C)NP(=O)(OCC(=O)N1CCN(Cc2ccccc2)C2(CCNC2=O)C1)OC(C)C] | ET-164 | 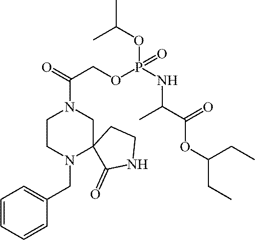 [SMILES: CCC(CC)OC(=O)C(C)NP(=O)(OCC(=O)N1CCN(Cc2ccccc2)C2(CCNC2=O)C1)OC(C)C] | ET-165 |
| 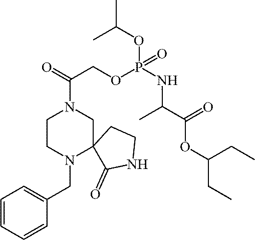 [SMILES: CCC(CC)OC(=O)C(C)NP(=O)(OCC(=O)N1CCN(Cc2ccccc2)C2(CCNC2=O)C1)OC(C)C] | ET-166 | 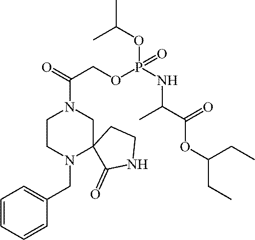 [SMILES: CCC(CC)OC(=O)C(C)NP(=O)(OCC(=O)N1CCN(Cc2ccccc2)C2(CCNC2=O)C1)OC(C)C] | ET-167 |
| 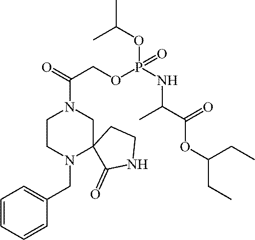 [SMILES: CCC(CC)OC(=O)C(C)NP(=O)(OCC(=O)N1CCN(Cc2ccccc2)C2(CCNC2=O)C1)OC(C)C] | ET-168 | 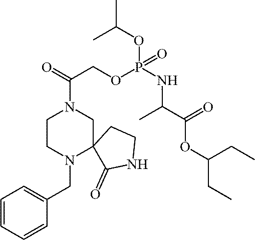 [SMILES: CCC(CC)OC(=O)C(C)NP(=O)(OCC(=O)N1CCN(Cc2ccccc2)C2(CCNC2=O)C1)OC(C)C] | ET-169 |
| 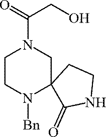 [SMILES: O=C(CO)N1CCN(Cc2ccccc2)C2(CCNC2=O)C1] | ET-170 | 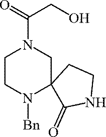 [SMILES: O=C(CO)N1CCN(Cc2ccccc2)C2(CCNC2=O)C1] | ET-171 |

The following are other exemplary compounds having Formula (A).

| Structure | Compound | Structure | Compound |
|---|---|---|---|
| 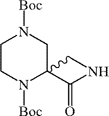 [SMILES: CC(C)(C)OC(=O)N1CCN(C(=O)OC(C)(C)C)C2(CNC2=O)C1] | EE-1 | 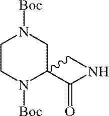 [SMILES: CC(C)(C)OC(=O)N1CCN(C(=O)OC(C)(C)C)C2(CNC2=O)C1] | EE-2 |
| 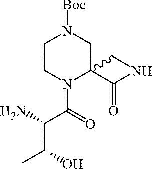 [SMILES: C[C@@H](O)[C@H](N)C(=O)N1CCN(C(=O)OC(C)(C)C)CC12CNC2=O] | EC-1 | 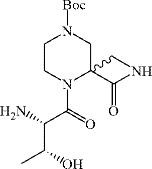 [SMILES: C[C@@H](O)[C@H](N)C(=O)N1CCN(C(=O)OC(C)(C)C)CC12CNC2=O] | EC-2 |
| 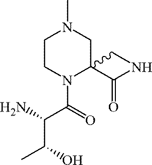 [SMILES: C[C@@H](O)[C@H](N)C(=O)N1CCN(C)CC12CNC2=O] | EJ-1 | 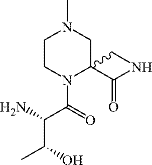 [SMILES: C[C@@H](O)[C@H](N)C(=O)N1CCN(C)CC12CNC2=O] | EJ-2 |
| 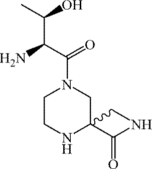 [SMILES: C[C@@H](O)[C@H](N)C(=O)N1CCNC2(CNC2=O)C1] | EK-1 | 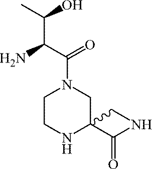 [SMILES: C[C@@H](O)[C@H](N)C(=O)N1CCNC2(CNC2=O)C1] | EK-2 |
| 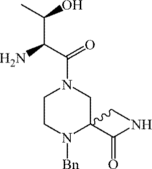 [SMILES: C[C@@H](O)[C@H](N)C(=O)N1CCN(Cc2ccccc2)C2(CNC2=O)C1] | EL-1 | 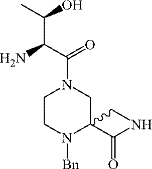 [SMILES: C[C@@H](O)[C@H](N)C(=O)N1CCN(Cc2ccccc2)C2(CNC2=O)C1] | EL-2 |
| 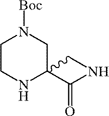 [SMILES: CC(C)(C)OC(=O)N1CCNC2(CNC2=O)C1] | EB-1 | 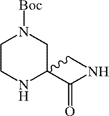 [SMILES: CC(C)(C)OC(=O)N1CCNC2(CNC2=O)C1] | EB-2 |
| 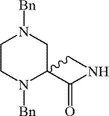 [SMILES: O=C1NCC12CN(Cc1ccccc1)CCN2Cc1ccccc1] | EA-1 | 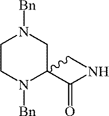 [SMILES: O=C1NCC12CN(Cc1ccccc1)CCN2Cc1ccccc1] | EA-2 |
| 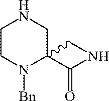 [SMILES: O=C1NCC12CNCCN2Cc1ccccc1] | ED-1 | 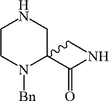 [SMILES: O=C1NCC12CNCCN2Cc1ccccc1] | ED-2 |
| 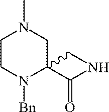 [SMILES: CN1CCN(Cc2ccccc2)C2(CNC2=O)C1] | EG-1 | 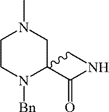 [SMILES: CN1CCN(Cc2ccccc2)C2(CNC2=O)C1] | EG-2 |
| 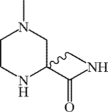 | EH-1 | 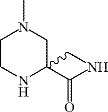 | EH-2 |
| 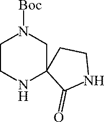 [SMILES: CC(C)(C)OC(=O)N1CCNC2(CCNC2=O)C1] | EM | 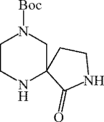 [SMILES: CC(C)(C)OC(=O)N1CCNC2(CCNC2=O)C1] | EN |

The compounds of the present disclosure and formulations thereof may have a plurality of chiral centers. Each chiral center may be independently R, S, or any mixture of R and S. For example, in some embodiments, a chiral center may have an R:S ratio of between about 100:0 and about 50:50 (“racemate”), between about 100:0 and about 75:25, between about 100:0 and about 85:15, between about 100:0 and about 90:10, between about 100:0 and about 95:5, between about 100:0 and about 98:2, between about 100:0 and about 99:1, between about 0:100 and 50:50, between about 0:100 and about 25:75, between about 0:100 and about 15:85, between about 0:100 and about 10:90, between about 0:100 and about 5:95, between about 0:100 and about 2:98, between about 0:100 and about 1:99, between about 75:25 and 25:75, and about 50:50. Formulations of the disclosed compounds comprising a greater ratio of one or more isomers (i.e., R and/or S) may possess enhanced therapeutic characteristic relative to racemic formulations of a disclosed compounds or mixture of compounds. In some instances, chemical formulas contain the descriptor “—(R)—” or “—(S)—” that is further attached to solid wedge or dashed wedge. This descriptor is intended to show a methine carbon (CH) that is attached to three other substituents and has either the indicated R or S configuration.

### Compositions

The present disclosure also provides a pharmaceutical formulation or a pharmaceutical composition including a disclosed compound and a pharmaceutically acceptable excipient for use in the methods of the invention. In some embodiments, a pharmaceutical composition comprises a racemic mixture of one or more of the disclosed compounds.

A formulation can be prepared in any of a variety of forms for use such as for administering an active agent to a patient, who may be in need thereof, as are known in the pharmaceutical arts. For example, the pharmaceutical compositions of the present disclosure can be formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, and pastes for application to the tongue; (2) parenteral administration by, for example, subcutaneous, intramuscular, intraperitoneal, intravenous or epidural injection as, for example, a sterile solution or suspension, or sustained-release formulation; (3) topical administration, for example, as a cream, ointment, or a controlled-release patch or spray applied to the skin; (4) intravaginal or intrarectal administration, for example, as a pessary, cream or foam; (5) sublingual administration; (6) ocular administration; (7) transdermal administration; or (8) nasal administration.

For example, pharmaceutical compositions of the disclosure can be suitable for delivery to the eye, i.e., ocularly. Related methods can include administering a pharmaceutically effective amount of a disclosed compound or a pharmaceutical composition including a disclosed compound to a patient in need thereof, for example, to an eye of the patient, where administering can be topically, subconjunctivally, subtenonly, intravitreally, retrobulbarly, peribulbarly, intracomerally, and/or systemically.

Amounts of a disclosed compound as described herein in a formulation may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

The specification for the dosage unit forms are dictated by and directly dependent on (a) the unique characteristics of the compound selected and the particular therapeutic effect to be achieved, and (b) the limitations inherent in the art of compounding such an active compound for the treatment of sensitivity in individuals.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

The compounds can be administered in a time release formulation, for example in a composition which includes a slow release polymer. The compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, a compound can be formulated with one or more additional compounds that enhance the solubility of the compound. In certain embodiments, pharmaceutical compositions described herein can be administered in combination with one or more additional therapeutic agents to treat a disorder described herein.

### Methods of Use and Treatment

Disclosed compounds can be used in methods of treating patients suffering from a viral infection, e.g., a coronaviral infection. In particular, in certain embodiments, the disclosure provides a method of treating the below medical indications comprising administering to a subject in need thereof a therapeutically effective amount of a compound described herein, such as a compound of Formula A, or a pharmaceutically acceptable salt thereof.

In some embodiments, the disclosure provides a method of ameliorating or treating a viral infection in a patient in need thereof, comprising administering to the patient a therapeutically effective amount of any of the compounds described herein. In some embodiments, the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenavirus, a herpes virus, and a hepatovirus. In certain embodiments, the viral infection is a coronavirus infection. In some embodiments, the viral infection is a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV2 (COVID-19). In embodiments, the viral infection is SARS-CoV2.

In embodiments, the viral infection is an arenavirus infection. In some embodiments, the arenavirus is selected from the group consisting of: Junin virus, Lassa virus, Lujo virus, Machupo virus, and Sabia virus. In some embodiments, the viral infection is an influenza infection. In some embodiments, the influenza is influenza H1N1, H3N2 or H5N1.

Also provided herein, in certain embodiments, is a method of inhibiting transmission of a virus, a method of inhibiting viral replication, a method of minimizing expression of viral proteins, or a method of inhibiting virus release, comprising administering a therapeutically effective amount of a compound described herein (e.g., a compound of Formula A) or a pharmaceutically acceptable salt thereof, to a patient suffering from the virus, and/or contacting an effective amount of a compound described herein (e.g., a compound of Formula A) or a pharmaceutically acceptable salt thereof, with a virally infected cell.

In some embodiments, the method further comprises administering another therapeutic. In some embodiments, the method further comprises administering an additional anti-viral therapeutic. In embodiments, the anti-viral therapeutic is selected from the group consisting of ribavirin, favipiravir, ST-193, oseltamivir, zanamivir, peramivir, danoprevir, ritonavir, and remdesivir. In some embodiments, the another therapeutic is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine. In embodiments, the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, and remdesivir.

Contemplated patients include not only humans, but other animals such as companion animals (e.g. dogs, cats), domestic animals, and wild animals (e.g. monkeys, bats, snakes).

Accordingly, in some embodiments, described herein is a method of ameliorating or treating a viral infection in a patient in need thereof, comprising administering to the patient a therapeutically effective amount of a compound described herein (e.g., a compound of Formula A described herein) or a pharmaceutically acceptable salt thereof.

Other contemplated methods of treatment include method of treating or ameliorating a virus infection condition or co-morbidity, by administering a compound disclosed herein to a subj ect.

Exemplary co-morbidities include lung diseases, cardiac disorders, endocrine disorders, respiratory disorders, hepatic disorders, skeletal disorders, psychiatric disorders, metabolic disorders, and reproductive disorders.

In some embodiments, the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenavirus, a herpes virus, and a hepatovirus. In some embodiments, the viral infection is a coronavirus infection. In some embodiments, the viral infection is a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV2 (COVID-19). In some embodiments, the viral infection is SARS-CoV2. In some embodiments, the viral infection is an arenavirus infection. In some embodiments, the arenavirus is selected from the group consisting of: Junin virus, Lassa virus, Lujo virus, Machupo virus, and Sabia virus. In some embodiments, the viral infection is an influenza infection. In some embodiments, the influenza is influenza H1N1, H3N2 or H5N1. In some embodiments, the viral infection is a respiratory viral infection. In some embodiments, the viral infection is an upper respiratory viral infection or a lower respiratory viral infection. In some embodiments, the method further comprises administering another therapeutic.

In certain embodiments, the virus is selected from the group consisting of a retrovirus (e.g., human immunodeficiency virus (HIV), simian immunodeficiency virus (SIV), human T-cell lymphotropic virus (HTLV)-1, HTLV-2, HTLV-3, HTLV-4), Ebola virus, hepatitis A virus, hepatitis B virus, hepatitis C virus, a herpes simplex virus (HSV) (e.g., HSV-1, HSV-2, varicella zoster virus, cytomegalovirus), an adenovirus, an orthomyxovirus (e.g., influenza virus A, influenza virus B, influenza virus C, influenza virus D, togavirus), a flavivirus (e.g., dengue virus, Zika virus), West Nile virus, Rift Valley fever virus, an arenavirus, Crimean-Congo hemorrhagic fever virus, an echovirus, a rhinovirus, coxsackie virus, a coronavirus (e.g., Severe acute respiratory syndrome coronavirus 2 (SARS-CoV2), coronavirus disease 2019 (COVID-19), a respiratory syncytial virus, a mumps virus, a rotavirus, measles virus, rubella virus, a parvovirus (e.g., an adeno-associated virus), a vaccinia virus, a variola virus, a molluscum virus, bovine leukemia virus, bovine diarrhea virus, a poliovirus, St. Louis encephalitis virus, Japanese encephalitis virus, a tick-borne encephalitis virus, Murray Valley virus, Powassan virus, Rocio virus, louping-ill virus, Banzi virus, Ilheus virus, Kokobera virus, Kunjin virus, Alfuy virus, a rabies virus, a polyomavirus (e.g., JC virus, BK virus), an alphavirus, and a rubivirus (e.g., rubella virus).

In certain embodiments, the disease or disorder is a viral infection, e.g., a disease or disorder selected from the group consisting of acquired immune deficiency syndrome (AIDS), HTLV-1 associated myelopathy/tropical spastic paraparesis, Ebola virus disease, hepatitis A, hepatitis B, hepatitis C, herpes, herpes zoster, acute varicella, mononucleosis, respiratory infections, pneumonia, influenza, dengue fever, encephalitis (e.g., Japanese encephalitis, St. Louis encephalitis, or tick-borne encephalitis such as Powassan encephalitis), West Nile fever, Rift Valley fever, Crimean-Congo hemorrhagic fever, Kyasanur Forest disease, Yellow fever, Zika fever, aseptic meningitis, myocarditis, common cold, lung infections, molloscum contagiosum, enzootic bovine leucosis, coronavirus disease 2019 (COVID-19), mumps, gastroenteritis, measles, rubella, slapped-cheek disease, smallpox, warts (e.g., genital warts), molluscum contagiosum, polio, rabies, and pityriasis rosea.

In some embodiments, the virus is an RNA virus (having a genome that is composed of RNA). RNA viruses may be single-stranded RNA (ssRNA) or double-stranded RNA (dsRNA). RNA viruses have high mutation rates compared to DNA viruses, as RNA polymerase lacks proofreading capability (see Steinhauer DA, Holland JJ (1987). “Rapid evolution of RNA viruses”. Annu. Rev. Microbiol. 41: 409-33). In some embodiments, the RNA virus is a positive-strand RNA virus (e.g., a SARS-CoV virus, polio virus, Coxsackie virus, Enterovirus, Human rhinovirus, Foot/Mouth disease virus, encephalomyocarditis virus, Dengue virus, Zika virus, Hepatitis C virus, or New Castle Disease virus).

RNA viruses are classified by the type of genome (double-stranded, negative (-), or positive (+) single-stranded). Double-stranded RNA viruses contain a number of different RNA molecules, each coding for one or more viral proteins. Positive-sense ssRNA viruses utilize their genome directly as mRNA; ribosomes within the host cell translate mRNA into a single protein that is then modified to form the various proteins needed for viral replication. One such protein is RNA-dependent RNA polymerase (RNA replicase), which copies the viral RNA in order to form a double-stranded, replicative form. Negative-sense ssRNA viruses have their genome copied by an RNA replicase enzyme to produce positive-sense RNA for replication. Therefore, the virus comprises an RNA replicase enzyme. The resultant positive-sense RNA then acts as viral mRNA and is translated by the host ribosomes. In some embodiments, the virus is a dsRNA virus. In some embodiments, the virus is a negative ssRNA virus. In some embodiments, the virus is a positive ssRNA virus. In some embodiments, the positive ssRNA virus is a coronavirus.

SARS-CoV2, also sometimes referred to as the novel coronavirus of 2019 or 2019-nCoV, is a positive-sense single-stranded RNA virus. SARS-CoV2 has four structural proteins, known as the S (spike), E (envelope), M (membrane), and N (nucleocapsid) proteins. The N protein holds the RNA genome together; the S, E, and M proteins form the viral envelope. Spike allows the virus to attach to the membrane of a host cell, such as the ACE2 receptor in human cells (Kruse R.L. (2020), Therapeutic strategies in an outbreak scenario to treat the novel coronavirus originating in Wuhan, China (version 2). F1000Research, 9:72). SARS-CoV2 is the highly contagious, causative viral agent of coronavirus disease 2019 (COVID19), a global pandemic

In some embodiments, the virus is a DNA virus (having a genome that is composed of DNA). Exemplary DNA viruses include, without limitation, parvoviruses (e.g., adeno-associated viruses), adenoviruses, asfarviruses, herpesviruses (e.g., herpes simplex virus 1 and 2 (HSV-1 and HSV-2), Epstein-Barr virus (EBV), cytomegalovirus (CMV)), papillomaviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). Exemplary RNA viruses include, without limitation, bunyaviruses (e.g., hantavirus), coronaviruses, flaviviruses (e.g., yellow fever virus, west Nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

The methods described herein may inhibit viral replication transmission, replication, assembly, or release, or minimize expression of viral proteins. In one embodiment, described herein is a method of inhibiting transmission of a virus, a method of inhibiting viral replication, a method of minimizing expression of viral proteins, or a method of inhibiting virus release, comprising administering a therapeutically effective amount of a compound described herein, or a pharmaceutically acceptable salt thereof, to a patient suffering from the virus, and/or contacting an effective amount of a compound described herein or a pharmaceutically acceptable salt thereof, with a virally infected cell.

In particular, in certain embodiments, the disclosure provides a method of treating the above medical indications comprising administering a subject in need thereof a therapeutically effective amount of a compound described herein, such as a disclosed compound.

Methods of the disclosure for treating a condition in a patient in need thereof include administering a therapeutically effective amount of a compound described herein or a composition including such a compound. In some embodiments, the condition may be a viral infection, e.g., a disease or disorder selected from the group consisting of acquired immune deficiency syndrome (AIDS), HTLV-1 associated myelopathy/tropical spastic paraparesis, Ebola virus disease, hepatitis A, hepatitis B, hepatitis C, herpes, herpes zoster, acute varicella, mononucleosis, respiratory infections, pneumonia, influenza, dengue fever, encephalitis (e.g., Japanese encephalitis, St. Louis encephalitis, or tick-borne encephalitis such as Powassan encephalitis), West Nile fever, Rift Valley fever, Crimean-Congo hemorrhagic fever, Kyasanur Forest disease, Yellow fever, Zika fever, aseptic meningitis, myocarditis, common cold, lung infections, molloscum contagiosum, enzootic bovine leucosis, coronavirus disease 2019 (COVID-19), mumps, gastroenteritis, measles, rubella, slapped-cheek disease, smallpox, warts (e.g., genital warts), molluscum contagiosum, polio, rabies, and pityriasis rosea.

Also provided herein are methods of treating a condition in treatment-resistant patients, e.g., patients suffering from a viral infenction that does not, and/or has not, responded to adequate courses of at least one, or at least two, other compounds or therapeutics. For example, provided herein is a method of treating a viral infenction in a treatment resistant patient, comprising a) optionally identifying the patient as treatment resistant and b) administering an effective dose of a compound to said patient.

Also provided herein are combination therapies comprising a compound described herein (e.g., a compound of Formula A) or a pharmaceutically acceptable salt thereof, in combination with one or more other active agents to treat a disorder described herein, such as an infection by a pathogen described herein, e.g., a virus, fungus, or protozoan. For clarity, contemplated herein are both a fixed composition comprising a disclosed compound and another therapeutic agent such as disclosed herein, and methods of administering, separately a disclosed compound and a disclosed therapeutic. For example, provided in the present disclosure is a pharmaceutical composition comprising a compound described herein (e.g., a compound of Formula A) or a pharmaceutically acceptable salt thereof, one or more additional therapeutic agents, and a pharmaceutically acceptable excipient.

In some embodiments, a compound described herein (e.g., a compound of Formula A) or a pharmaceutically acceptable salt thereof, and one additional therapeutic agent is administered. In some embodiments, a disclosed compound as defined herein and two additional therapeutic agents are administered. In some embodiments, a disclosed compound as defined herein and three additional therapeutic agents are administered. Combination therapy can be achieved by administering two or more therapeutic agents, each of which is formulated and administered separately. For example, a compound described herein (e.g., a compound of Formula A) or a pharmaceutically acceptable salt thereof, and an additional therapeutic agent can be formulated and administered separately.

Combination therapy can also be achieved by administering two or more therapeutic agents in a single formulation, for example a pharmaceutical composition comprising a compound described herein (e.g., a compound of Formula A) or a pharmaceutically acceptable salt thereof, as one therapeutic agent and one or more additional therapeutic agents such as an antibiotic, a viral protease inhibitor, or an anti-viral nucleoside anti-metabolite. For example, a compound described herein (e.g., a compound of Formula A) or a pharmaceutically acceptable salt thereof, and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient’s body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

In some embodiments, the one or more additional therapeutic agents that may be administered in combination with a compound provided herein can be an antibiotic, a viral protease inhibitor, an anti-viral anti-metabolite, a lysosomotropic agent, a M2 proton channel blocker, a polymerase inhibitor (e.g., EIDD-2801), a neuraminidase inhibitor, a reverse transcriptase inhibitor, a viral entry inhibitor, an integrase inhibitor, interferons (e.g., types I, II, and III), or a nucleoside analogue.

In some embodiments, methods described herein further comprise administering an additional anti-viral therapeutic. In some embodiments, the anti-viral therapeutic is selected from the group consisting of ribavirin, favipiravir, ST-193, oseltamivir, zanamivir, peramivir, danoprevir, ritonavir, and remdesivir. In some embodiments, the another therapeutic is selected from the group consisting of protease inhibitors (e.g., nafamostat, camostat, gabexate, epsilon-aminocapronic acid and aprotinin), fusion inhibitors (e.g., BMY-27709, CL 61917, and CL 62554), M2 proton channel blockers (e.g., amantadine and rimantadine), polymerase inhibitors (e.g., 2-deoxy-2′fluoroguanosides (2′-fluoroGuo), 6- endonuclease inhibitors (e.g., L-735,822 and flutamide) neuraminidase inhibitors (e.g., zanamivir (Relenza), oseltamivir, peramivir and ABT-675 (A-315675), reverse transcriptase inhibitor (e.g., abacavir, adefovir, delavirdine, didanosine, efavirenz, emtricitabine, lamivudine, nevirapine, stavudine, tenofovir, tenofovir disoproxil, and zalcitabine), acyclovir, acyclovir, protease inhibitors (e.g., amprenavir, indinavir, nelfinavir, ritonavir, and saquinavir), arbidol, atazanavir, atripla, boceprevir, cidofovir, combivir, darunavir, docosanol, edoxudine, entry inhibitors (e.g., enfuvirtide and maraviroc), entecavir, famciclovir, fomivirsen, fosamprenavir, foscarnet, fosfonet, ganciclovir, ibacitabine, immunovir, idoxuridine, imiquimod, inosine, integrase inhibitor (e.g., raltegravir), interferons (e.g., types I, II, and III), lopinavir, loviride, moroxydine, nexavir, nucleoside analogues (e.g., aciclovir), penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine. In some embodiments, the additional anti-viral therapeutic is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, and remdesivir. In some embodiments, the another therapeutic is selected from the group consisting of quinine (optionally in combination with clindamycin), chloroquine, amodiaquine, artemisinin and its derivatives (e.g., artemether, artesunate, dihydroartemisinin, arteether), doxycycline, pyrimethamine, mefloquine, halofantrine, hydroxychloroquine, eflornithine, nitazoxanide, omidazole, paromomycin, pentamidine, primaquine, pyrimethamine, proguanil (optionally in combination with atovaquone), a sulfonamide (e.g., sulfadoxine, sulfamethoxypyridazine), tafenoquine, tinidazole and a PPT1 inhibitor (including Lys05 and DC661). In some embodiments, the another therapeutic is an antibiotic. In some embodiments, the antibiotic is a penicillin antibiotic, a quinolone antibiotic, a tetracycline antibiotic, a macrolide antibiotic, a lincosamide antibiotic, a cephalosporin antibiotic, or an RNA synthetase inhibitor. In some embodiments, the antibiotic is selected from the group consisting of azithromycin, vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and streptomycin. In some embodiments, the antibiotic is azithromycin.

In some embodiments, the additional therapeutic agents can be kinase inhibitors including but not limited to erlotinib, gefitinib, neratinib, afatinib, osimertinib, lapatanib, crizotinib, brigatinib, ceritinib, alectinib, lorlatinib, everolimus, temsirolimus, abemaciclib, LEE011, palbociclib, cabozantinib, sunitinib, pazopanib, sorafenib, regorafenib, sunitinib, axitinib, dasatinib, imatinib, nilotinib, ponatinib, idelalisib, ibrutinib, Loxo 292, larotrectinib, and quizartinib.

In some embodiments, the additional therapeutic agents can be therapeutic anti-viral vaccines.

In some embodiments, the additional therapeutic agents can be immunomodulatory agents including but not limited to anti-PD-1or anti-PDL-1 therapeutics including pembrolizumab, nivolumab, atezolizumab, durvalumab, BMS-936559, or avelumab, anti-TIM3 (anti-HAVcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or RO7009789, anti-CD47 therapeutics including but not limited to Hu5F9-G4, anti-CD20 therapeutics, anti-CD38 therapeutics, STING agonists including but not limited to ADU-S100, MK-1454, ASA404, or amidobenzimidazoles, anthracyclines including but not limited to doxorubicin or mitoxanthrone, hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone.

### Conjugates

In some embodiments, the conjugate, which can be a reversible conjugate, represented by:

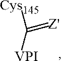

wherein Cys₁₄₅ is cysteine at position 145 or equivalent active site cysteine on Mpro, for example, a CoV Mpro; Z′ is O, S or NH; and VPI is a viral protease inhibitor.

In other embodiments, the reversible conjugate represented by:

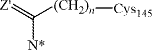

wherein: Cys₁₄₅ is cysteine at position 145 or equivalent active site cysteine on Mpro, for example, a CoV Mpro; Z′ is O, S or NH; n is independently, for each occurrence, 0, 1 or 2; and N* is a ring nitrogen of a compound, or a pharmaceutically acceptable salt and/or a stereoisomer thereof, wherein N* comprises the compound, or a pharmaceutically acceptable salt and/or a stereoisomer thereof, and the compound is a compound having Formula (A).

For example, a conjugate can be represented by:

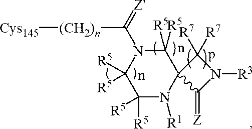

wherein the variables are as defined herein with respect to compounds of Formula (A).

In certain embodiments, each of n is 1. In particular embodiments, p is 2. In some embodiments, Z and Z′ are O.

In particular embodiments, the conjugate is represented by:

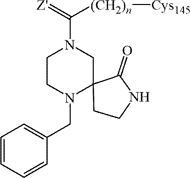

wherein Z′ and n are as defined herein for the compounds of Formula (A).

In some embodiments, Z′ is O. In certain embodiments, n is 1.

In certain embodiments, a conjugate represented by:

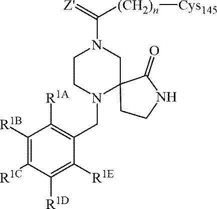

wherein Z′ is O, S or NH;
n is independently, for each occurrence, 0, 1 or 2; and
one, two or three of R^{1A}, R^{1B}, R^{1C}, R^{1D}, and R^{1E} are optionally each independently selected from the group consisting of H, hydroxyl, halogen, —C(O)—O—C₁—C₃alkyl, —C(O)—C₁—C₃alkyl, methyl, and CF₃.

### EXAMPLES

The compounds of Formula (A), as disclosed herein, as well as their pharmaceutically acceptable salts, can be prepared by methods known from the literature. See, for example, International Application Publication No. WO 2018/026782 A1, which is incorporated by reference herein.

The following abbreviations may be used herein and have the indicated definitions: AIDS is acquired immune deficiency syndrome, Boc and BOC are tert-butoxycarbonyl, Boc₂O is di-tert-butyl dicarbonate, Bn is benzyl, BOM-Cl is benzyloxymethyl chloride, CAN is ceric ammonium nitrate, Cbz is carboxybenzyl, DCM is dichloromethane, DIAD is diisopropyl azodicarboxylate, DIPEA is N,N-diisopropylethylamine, DMAP is 4-dimethylaminopyridine, DMF is N,N-dimethylformamide, DMSO is dimethyl sulfoxide, EDC and EDCI are 1-ethyl-3-(3-dimethylaminopropyl)carbodiimide hydrochloride, ESI is electrospray ionization, EtOAc is ethyl acetate, Gly is glycine, h is hour, HATU is 2-(7-aza-1H-benzotriazole-1-yl)-1,1,3,3-tetramethyluronium hexafluorophosphate, HIV is human immunodeficiency virus, HPLC is high performance liquid chromatography, LCMS is liquid chromatography/mass spectrometry, LiHMDS is lithium hexamethyldisilazane, MTBE is methyl tert-butyl ether, NMDAR is N-methyl-d-aspartate receptor, NMP is N-methyl-2-pyrrolidone, NMR is nuclear magnetic resonance, Pd/C is palladium on carbon, PMB is para-methoxybenzyl, RT is room temperature (e.g., from about 20° C. to about 25° C.), TBS and TBDMS are tert-butyldimethylsilyl, TEA is triethylamine, TLC is thin layer chromatography, TFA is trifluoroacetic acid, THF is tetrahydrofuran, TMS is trimethylsilyl, TMSCN is trimethylsilyl cyanide, and TPP is triphenylphosphine.

### A. Synthesis of Compounds

### Synthesis of ET-103 and ET-104

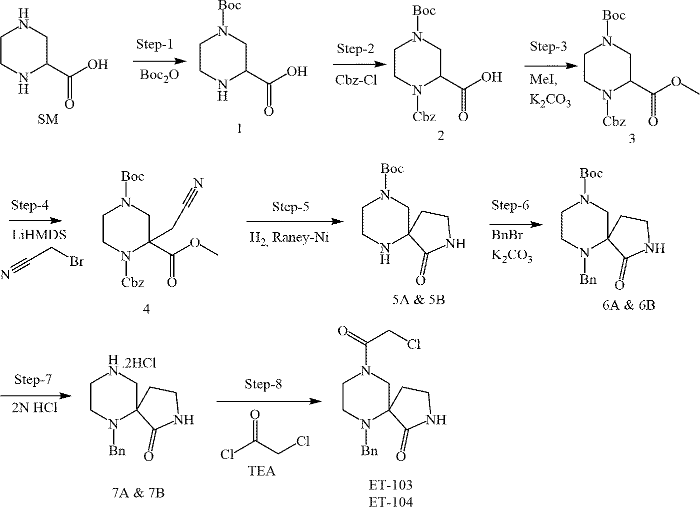

### Synthesis of 4-(Tert-Butoxycarbonyl)Piperazine-2-Carboxylic Acid (1)

To a stirred suspension of piperazine-2-carboxylic acid (SM) (500 g, 3.846 mol) in 1,4-dioxane:water (1:1, 8 L) was added NaHCO₃ (484 g, 5.769 mol) followed by Boc-anhydride (1.06 L, 4.615 mol) at 0° C. under nitrogen atmosphere. The reaction mixture was brought to room temperature and stirred for 48 h. After consumption of the starting material (by TLC), Et₂O (2 L) was added to the reaction mixture and organic layer was separated. Volatiles were reduced under pressure to afford compound 1 (~884 g in 4 L solvent). The crude was taken to next step without any further purification.

¹H-NMR (500 MHz, DMSO-d₆): δ 10.16 (br s, 1 H), 4.04 (br s, 1 H), 3.85-3.74 (m, 2 H), 3.63 (t, J= 6.5 Hz, 1 H), 3.19-3.16 (m, 2 H), 2.90-2.8 (m, 1 H), 1.38 (s, 9 H), 1.31-0.84 (m, 1 H).

LCMS (ESI): m/z 229.0 [M-H]⁻

### Synthesis of 1-((Benzyloxy)Carbonyl)-4-(Tert-Butoxycarbonyl)Piperazine-2-Carboxylic Acid (2)

To a stirring solution of crude compound 1 (884 g, 3.843 mol) in 1,4-dioxane:water (1:4, 5 L) was added NaHCO₃ (484 g, 5.765 mol) followed by drop wise addition of Cbz-Cl (50% in toluene) (784 g, 4.612 mol) at 0° C. The reaction mixture was brought to room temperature and stirred for 16 h. After consumption of the starting material (by TLC), the reaction was diluted with water (500 mL) and washed with Et₂O (500 mL). Aqueous layer was acidified with 2N HCl solution (pH = ~2) at 0-10° C. and extracted with EtOAc (3 x 500 mL). The organic layer was dried over anhydrous Na₂SO₄, concentrated under reduced pressure to afford compound 2 (940 g, 67 %) as thick brown viscous liquid.

¹H-NMR (400 MHz, DMSO-d₆): δ 13.05 (br s, 1H), 7.38-7.31 (m, 5H), 5.13-5.05 (m, 2H), 4.56-4.53 (m, 1H), 4.38-4.32 (m, 1H), 3.86-3.76 (m, 2H), 3.18-3.08 (m, 2H), 2.84 (br s, 1H), 1.37 (s, 9H).

### Synthesis of 1-Benzyl 4-(Tert-Butyl) 2-Methyl Piperazine-1,2,4-Tricarboxylate (3)

To a stirring solution of compound 2 (400 g, 1.098 mol) in DMF (1.2 L) were added K₂CO₃ (227 g, 1.648 mol) at 0° C. under nitrogen atmosphere. After stirring for 10 min, MeI (85 mL, 1.318 mol) was added drop wise. The reaction mixture was stirred at 0° C. for 1 h and at room temperature for 2 h. After consumption of the starting material (by TLC), the reaction was diluted with water (1 L) and extracted with Et₂O (2 x 1 L). Combined organic layer was washed with brine solution (500 mL), dried over Na₂SO₄ and concentrated under evaporated pressure. The obtained crude material was washed with 30% Et₂O in hexanes and dried under vacuum afford compound 3 (255 g, 61%) as white solid.
¹H NMR (400 MHz, DMSO-d₆): δ 7.44 - 7.21 (m, 5H), 5.20 - 4.98 (m, 2H), 4.78 - 4.63 (m, 1H), 4.31 (br t, J= 15.4 Hz, 1H), 3.98 - 3.73 (m, 2H), 3.65 (s, 3H), 3.26 - 3.00 (m, 2H), 2.96 -2.79 (m, 1H), 1.37 (s, 9H)
LCMS (ESI): m/z 279.1 [M-Boc+H]⁺

### Synthesis of 1-Benzyl 4-(Tert-Butyl) 2-Methyl 2-(Cyanomethyl)Piperazine-1,2,4-Tricarboxylate (4)

To a stirring solution of compound 3 (100 g, 0.264 mol) in THF (1 L) was added LiHMDS (1 M in THF, 396 mL, 0.396 mol) at -78° C. under nitrogen atmosphere. The reaction mixture was allowed to warm to -40° C. and stirred for 1.5 h. Again the reaction mixture was cooled to -78° C., bromo acetonitrile (27.7 mL, 0.396 mol) was added drop wise. The reaction mixture was allowed to warm to 0° C. and stirred for 3 h. After consumption of the starting material (60% by TLC), reaction mixture was quenched with NH₄Cl solution (200 mL) and extracted with EtOAc (2 x 500 mL). Combined organic layers were washed with brine solution (100 mL), dried over Na₂SO₄ and concentrated under evaporated pressure. The crude material was purified by column chromatography by eluting 10-20% EtOAc/ hexane to afford compound 4 (30 g, 27%) as viscous liquid.

¹H NMR (400 MHz, DMSO-d₆): δ 7.47-7.26 (m, 5H), 5.14 (br s, 2H), 4.00 (br d, J = 14.2 Hz, 2H), 3.85 (br s, 1 H), 3.72-3.53 (m, 3H), 3.42 (br s, 3H), 3.21 (s, 1H), 3.17 (d, J= 5.2 Hz, 1H), 1.38 (s, 9H).

LCMS (ESI): m/z 418.4 [M+H]⁺

### Synthesis of Tert-Butyl 1-Oxo-2,6,9-Triazaspiro[4.5]Decane-9-Carboxylate (5A & 5B)

To a stirring solution of compound 4 (10 g, 0.023 mol) in MeOH (150 mL) was added Raney Nickel (20 g) at room temperature under nitrogen atmosphere. The reaction mixture was stirred at room temperature for 48 h under H₂ atmosphere (20 kg). After consumption of the starting material (by TLC), the reaction mixture was filtered through a pad of celite and the filtrate was concentrated under reduced pressure. Obtained crude material was purified by column chromatography by eluting with 5% MeOH/ CH₂Cl₂ to afford racemic compound 5 (4 g, 66%) as white solid. The racemic product (4 g) was separated by chiral preparative HPLC purification to obtain compound 5A (1.4 g) as an off white solid and compound 5B (1.2 g) as an off white solid.

### 5A

¹H NMR (400 MHz, DMSO-d₆): δ 7.76 (br s, 1H), 3.68 - 3.49 (m, 2H), 3.26 - 3.16 (m, 1H), 3.14 - 3.04 (m, 1H), 2.95 - 2.77 (m, 3H), 2.59 - 2.52 (m, 1H), 2.15 - 2.10 (m, 1H), 2.04 - 1.98 (m, 1H), 1.85 - 1.78 (m, 1H), 1.39 (s, 9H)
LCMS (ESI): m/z 256.0 [M+H]⁺
HPLC: 93.63%
Chiral HPLC: >99.00%
Column : CHIRALPAK IG (250*4.6 mm*5 µm)
Mobile Phase : A: 0.1% DEA in n-Hexane
Mobile Phase : B: ETOH:MEOH(1:1)
A : B :: 75 : 25; Flow rate : 1.0 mL/min
Retention time : 12.671 min

### 5B

¹H NMR (400 MHz, DMSO-d₆): δ 7.76 (br s, 1H), 3.67 - 3.46 (m, 2H), 3.25 - 3.16 (m, 1H), 3.14 - 3.02 (m, 1H), 2.95 - 2.75 (m, 3H), 2.59 - 2.52 (m, 1H), 2.15 - 2.10 (m, 1H), 2.07 - 1.95 (m, 1H), 1.85 - 1.77 (m, 1H), 1.39 (s, 9H)
LCMS (ESI): m/z 256.0 [M+H]⁺
HPLC: 98.60%
Chiral HPLC: >99.00%
Column : CHIRALPAK IG (250*4.6 mm*5 µm)
Mobile Phase : A: 0.1% DEA in n-Hexane
Mobile Phase : B: ETOH:MEOH(1:1)
A : B :: 75 : 25; Flow rate : 1.0 mL/min
Retention time : 16.549 min

### Synthesis of Tert-Butyl 6-Benzyl-1-Oxo-2,6,9-Triazaspiro[4.5]decane-9-Carboxylate (6A)

To a stirring solution of compound 5A (1.4 g, 5.49 mmol) in CH₃CN (14 mL) were added K₂CO₃ (2.27 g, 16.47 mmol) and BnBr (1.4 mL, 8.23 mmol) at room temperature. The reaction mixture was stirred at room temperature for 16 h. After consumption of the starting material (by TLC), the reaction mixture was diluted with EtOAc (100 mL) and filtered through a pad of celite. Obtained filtrate was concentrated under reduced pressure. The crude material was triturated with Et₂O (50 mL) and dried under vacuum to afford compound 6A (1.6 g, 88%) as white solid.

### 6A

¹H NMR (400 MHz, DMSO-d₆): δ 7.92 (s, 1H), 7.39 (d, J= 7.3 Hz, 2H), 7.30 (t, J= 7.3 Hz, 2H), 7.25 - 7.19 (m, 1H), 3.82 - 3.61 (m, 2 H), 3.46 (d, J= 13.3 Hz, 1H), 3.26 (br d, J= 8.6 Hz, 1H), 3.21 - 3.12 (m, 2H), 3.02 - 2.72 (m, 2H), 2.46 (br dd, J= 2.8, 11.9 Hz, 1H), 2.18 - 2.05 (m, 2H), 1.88 (br dd, J = 7.2, 12.2 Hz, 1H), 1.40 (s, 9H)
LCMS (ESI): m/z 346.4 [M+H]⁺
HPLC: 98.18%
Chiral HPLC: 98.11%
Column : Chiralpak IC (150 X4.6 mm,3 µm)
Mobile Phase : A: 0.1% DEA in n-Hexane
Mobile Phase : B: DCM:MEOH (1:1)
A : B :: 75 : 25; Flow rate : 0.7 mL/min
Retention time : 16.314 min

### Synthesis of Tert-Butyl 6-Benzyl-1-Oxo-2,6,9-Triazaspiro[4.5]Decane-9-Carboxylate (6B)

To a stirring solution of compound 5B (1.2 g, 4.70 mmol) in CH₃CN (12 mL) were added K₂CO₃ (1.9 g, 14.11 mmol) and BnBr (0.83 mL, 7.05 mmol) at room temperature. The reaction mixture was stirred at room temperature for 16 h. After consumption of the starting material (by TLC), the reaction mixture was diluted with EtOAc (100 mL) and filtered through a pad of celite. Obtained filtrate was concentrated under reduced pressure. The crude material was triturated with Et₂O (50 mL) and dried under vacuum to afford compound 6B (1.4 g, 87%) as white solid.

### 6B

¹H NMR (400 MHz, DMSO-d₆): δ 7.92 (s, 1H), 7.39 (d, J= 7.3 Hz, 2H), 7.30 (t, J= 7.4 Hz, 2H), 7.25 - 7.19 (m, 1H), 3.80 - 3.62 (m, 2H), 3.46 (d, J= 13.3 Hz, 1H), 3.26 (br d, J= 8.6 Hz, 1H), 3.21 - 3.12 (m, 2H), 3.03 - 2.72 (m, 2H), 2.46 (br dd, J = 2.8, 11.9 Hz, 1H), 2.19 - 2.04 (m, 2H), 1.88 (br dd, J = 7.2, 12.3 Hz, 1H), 1.40 (s, 9H)
LCMS (ESI): m/z 346.2 [M+H]⁺
HPLC: 99.50%
Chiral HPLC: >99.00%
Column : Chiralpak IC (150 X4.6 mm,3 µm)
Mobile Phase : A: 0.1% DEA in n-Hexane
Mobile Phase : B: DCM:MEOH (1:1)
A : B :: 75 : 25; Flow rate : 0.7 mL/min
Retention time : 7.597 min

### Synthesis of 6-Benzyl-2,6,9-Triazaspiro[4.5]Decan-1-One Dihydrogen Chloride (7A)

To a stirring solution of compound 6A (1.6 g, 4.63 mmol) in CH₂Cl₂ (16 mL) was added 2N HCl in Et₂O (22 mL, 46.3 mmol) at 0° C. The reaction mixture was stirred at room temperature for 16 h. After consumption of the starting material (by TLC), volatiles were removed under reduced pressure. The crude material was triturated with ether (2x20 mL) and dried under vacuum to afford compound 7A (1.4 g, 95%) as white solid.

### 7A

¹H NMR (400 MHz, D₂O): δ 7.50 (s, 5H), 4.04 - 3.95 (m, 1H), 3.91 - 3.82 (m, 1H), 3.70 - 3.56 (m, 3H), 3.55 - 3.48 (m, 1H), 3.45 - 3.31 (m, 3H), 3.11 - 2.97 (m, 1 H), 2.75 (ddd, J= 6.0, 8.8, 14.8 Hz, 1H), 2.51 (ddd, J = 4.7, 8.5, 14.7 Hz, 1H)
LCMS (ESI): m/z 246.2 [M+H]⁺
HPLC: 98.56%
Chiral HPLC: >99.00%
Column : CHIRALPAK IA (250*4.6 mm*5 µm)
Mobile Phase : A: 0.1% DEA in n-Hexane
Mobile Phase : B: DCM:MEOH (1:1)
A : B :: 75 : 25; Flow rate : 1.0 mL/min
Retention time : 7.332 min

### Synthesis of 6-benzyl-2,6,9-triazaspiro[4.5]decan-1-one Dihydrogen Chloride (7B)

To a stirring solution of compound 6B (1.4 g, 4.05 mmol) in CH₂Cl₂ (14 mL) was added 2N HCl in Et₂O (22 mL, 40.5 mmol) at 0° C. The reaction mixture was stirred at room temperature for 16 h. After consumption of the starting material (by TLC), volatiles were removed under reduced pressure. The crude material was triturated with ether (2x20 mL) and dried under vacuum to afford compound 7B (1.2 g, 93%) as white solid.

### 7B:

¹H NMR (400 MHz, D₂O): δ 7.51 (s, 5H), 4.01 - 3.94 (m, 1H), 3.91 - 3.81 (m, 1H), 3.70 - 3.57 (m, 3H), 3.55 - 3.48 (m, 1H), 3.51 - 3.33 (m, 3H), 3.19 - 3.06 (m, 1H), 2.79 (ddd, J = 6.5, 8.4, 14.7 Hz, 1H), 2.50 (ddd, J = 4.7, 8.5, 14.7 Hz, 1H)
LCMS (ESI): m/z 246.0 [M+H]⁺
HPLC: 94.60%
Chiral HPLC: 97.54%
Column : CHIRALPAK IA (250*4.6 mm*5 µm)
Mobile Phase : A: 0.1% DEA in n-Hexane
Mobile Phase : B: DCM:MEOH (1:1)
A : B :: 75 : 25; Flow rate : 1.0 mL/min
Retention time : 5.853 min

### Synthesis of 6-benzyl-9-(2-chloroacetyl)-2,6,9-triazaspiro[4.5]decan-1-one (ET-103)

To a stirring solution of compound 7A (1.4 g, 4.40 mmol) in CH₂Cl₂ (14 mL) were added Et₃N (1.83 mL, 13.20 mmol) and 2-chloroacetyl chloride (0.53 mL, 6.60 mmol) at 0° C. under nitrogen atmosphere. The reaction mixture was stirred at room temperature for 3 h. After consumption of the starting material (by TLC), the reaction was diluted with water (10 mL) and extracted with Et₂O (2 x 50 mL). The organic layer was dried over anhydrous Na₂SO₄, concentrated under reduced pressure. The crude material was purified by medium pressure liquid chromatography by eluting 2-5% MeOH/ CH₂Cl₂ to afford ET-103 (700 mg, 50%) as white solid.
¹HNMR (400 MHz, DMSO-d₆): δ 8.05 - 7.88 (m, 1 H), 7.44 - 7.36 (m, 2H), 7.31 (t, J= 7.4 Hz, 2H), 7.26 - 7.20 (m, 1H), 4.50 - 4.39 (m, 1H), 4.36 - 4.24 (m, 1H), 4.12 - 3.93 (m, 1H), 3.75 -3.54 (m, 1H), 3.52 - 3.39 (m, 1H), 3.29 - 3.08 (m, 4H), 2.83 (br d, J= 12.1 Hz, 1H), 2.58 - 2.52 (m, 1H), 2.31 - 1.77 (m, 3H)
LCMS (ESI): m/z 322.1 [M+H]⁺
HPLC: 99.57%
Chiral HPLC: >99.00%
Column : CHIRALPAK IA (250*4.6 mm*5 µm)
Mobile Phase : A: 0.1% DEA in n-Hexane
Mobile Phase : B: ETOH
A : B :: 75 : 25; Flow rate : 1.0 mL/min
Retention time : 9.283 min

### Synthesis of 6-benzyl-9-(2-chloroacetyl)-2,6,9-triazaspiro[4.5]decan-1-one (ET-104)

To a stirring solution of compound 7A (1.2 g, 3.77 mmol) in CH₂Cl₂ (12 mL) were added Et₃N (1.5 mL, 11.32 mmol) and 2-chloroacetyl chloride (0.45 mL, 5.66 mmol) at 0° C. under nitrogen atmosphere. The reaction mixture was stirred at room temperature for 3 h. After consumption of the starting material (by TLC), the reaction was diluted with water (10 mL) and extracted with Et₂O (2 x 50 mL). The organic layer was dried over anhydrous Na₂SO₄, concentrated under reduced pressure. The crude material was purified by medium pressure liquid chromatography by eluting 2-5% MeOH/ CH₂Cl₂ to afford ET-104 (700 mg, 58%) as white solid.
¹HNMR (400 MHz, DMSO-d₆): δ 8.05 - 7.88 (m, 1H), 7.44 - 7.36 (m, 2H), 7.31 (t, J= 7.4 Hz, 2H), 7.26 - 7.20 (m, 1H), 4.50 - 4.39 (m, 1H), 4.36 - 4.24 (m, 1H), 4.12 - 3.93 (m, 1H), 3.75 -3.54 (m, 1H), 3.52 - 3.39 (m, 1H), 3.29 - 3.08 (m, 4H), 2.83 (br d, J= 12.1 Hz, 1H), 2.58 - 2.52 (m, 1H), 2.31 - 1.77 (m, 3H)
LCMS (ESI): m/z 322.1 [M+H]⁺
HPLC: 99.02%
Chiral HPLC: >99.00%
Column : CHIRALPAK IA (250*4.6 mm*5 µm)
Mobile Phase : A: 0.1% DEA in n-Hexane
Mobile Phase : B: ETOH
A : B :: 75 : 25; Flow rate : 1.0 mL/min
Retention time : 10.036 min

### Synthesis of ET-107 and ET-108

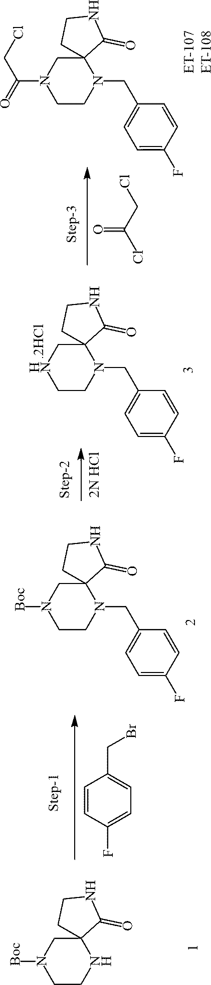

The experimental procedure for the synthesis of compound 1 is captured under ET-103 and ET-104 as racemic mixture of compounds 5A and 5B.

### Synthesis of Tert-butyl 6-(4-fluorobenzyl)-1-oxo-2,6,9-triazaspiro[4.5]decane-9-carboxylate (2)

To a stirring solution of compound 1 (1.5 g, 5.88 mmol) in CH₃CN (15 mL) were added K₂CO₃ (2.4 g, 17.64 mmol) followed by 1-(bromomethyl)-4-fluorobenzene (1.65 g, 8.82 mmol) at room temperature. The reaction mixture was stirred at room temperature for 16 h. After consumption of the starting material (by TLC), the reaction mixture was filtered through a pad of celite. Obtained filtrate was concentrated under reduced pressure. The crude material was triturated with Et₂O (5 mL) and dried under vacuum to afford compound 2 (1.8 g, 85%) as white solid.
¹H NMR (400 MHz, DMSO-d₆): δ 7.94 (s, 1H), 7.43 (dd, J = 5.9, 8.3 Hz, 2H), 7.12 (t, J = 8.9 Hz, 2H), 3.83 - 3.59 (m, 2H), 3.41 (br d, J= 13.3 Hz, 1H), 3.26 (br d, J= 8.8 Hz, 1H), 3.21 -3.12 (m, 2H), 3.03 - 2.70 (m, 2H), 2.42 (br d, J = 11.6 Hz, 1H), 2.20 - 2.00 (m, 2H), 1.88 (br dd, J= 7.0, 12.3 Hz, 1H), 1.40 (s, 9H)
LCMS (ESI): m/z 364.1 [M+H]⁺

### Synthesis of 1-(bromomethyl)-4-fluorobenzene Dihydrogen Chloride (3)

To a stirring solution of compound 2 (2 g, 5.51 mmol) in CH₂Cl₂ (20 mL) was added 2N HCl in Et₂O (5 mL, 11.01 mmol) at 0° C. The reaction mixture was stirred at room temperature for 16 h. After consumption of the starting material (by TLC), volatiles were removed under reduced pressure. The crude material was triturated with ether (2x20 mL) and dried under vacuum to afford compound 3 (1.4 g, 95%) as white solid.

¹H NMR (400 MHz, DMSO-d₆): δ 9.85 (br d, J= 1.1 Hz, 2H), 8.55 (br s, 1H), 7.59 - 7.34 (m, 3H), 7.20 (t, J= 8.8 Hz, 2H), 3.63 (br d, J= 13.0 Hz, 1H), 3.47 (br d, J= 12.5 Hz, 1H), 3.34 (br t, J = 6.7 Hz, 2H), 3.23 - 2.97 (m, 4H), 2.93 - 2.60 (m, 3H), 2.42 (br d, J = 1.8 Hz, 1H) LCMS (ESI): m/z 264.0 [M+H]⁺

### Synthesis of 9-(2-chloroacetyl)-6-(4-fluorobenzyl)-2,6,9-triazaspiro[4.5]decan-1-one (ET-107 & ET-108)

To a solution of compound 3 (1.5 g, 4.47 mmol) in CH₂Cl₂ (15 mL) was added Et₃N (1.9 mL, 13.43 mmol) at 0° C. under nitrogen atmosphere. After stirring for 10 min, 2-chloroacetyl chloride (0.53 mL, 6.71 mmol) was added. The reaction mixture was stirred at room temperature for 4 h. After consumption of the starting material (by TLC), the reaction was quenched with water (10 mL) and extracted with Et₂O (2 x 50 mL). The organic layer was dried over anhydrous Na₂SO₄, concentrated under reduced pressure. The crude material was purified by medium pressure liquid chromatography by eluting 2-3% MeOH/ CH₂Cl₂ to afford racemic ET-107 & ET-108 (1 g, 66%) as an off white solid. This material was further purified by chiral preparative HPLC purification to obtain ET-107 (180 mg) as white solid and ET-108 (180 mg) as white solid.
ET-107
   ¹H NMR (400 MHz, DMSO-d₆): δ 8.09 - 7.92 (m, 1H), 7.52 - 7.38 (m, 2H), 7.15 (t, J = 8.9 Hz, 2H), 4.52 - 4.41 (m, 1H), 4.39 - 4.27 (m, 1H), 4.15 - 3.97 (m, 1H), 3.76 - 3.55 (m, 1H), 3.48 -3.39 (m, 1H), 3.29 - 3.08 (m, 4H), 2.81 (br d, J= 11.6 Hz, 1H), 2.38 - 2.23 (m, 1H), 2.22 - 1.71 (m, 3H).
   LCMS (ESI): m/z 340.1 [M+H]⁺
   HPLC: 99.95%
   Chiral HPLC: >99.00%
   Column : CHIRALPAK IG (250*4.6 mm*5 µm)
   Mobile Phase : A: n-Hexane
   Mobile Phase : B: IPA
   A : B :: 50 : 50; Flow rate : 1.0 mL/min
   Retention time : 9.811 min
ET-108
   ¹H NMR (400 MHz, DMSO-d₆): δ 8.08 - 7.91 (m, 1H), 7.52 - 7.38 (m, 2H), 7.16 (t, J= 8.9 Hz, 2H), 4.52 - 4.40 (m, 1H), 4.38 - 4.27 (m, 1H), 4.15 - 3.98 (m, 1H), 3.76 - 3.55 (m, 1H), 3.48 -3.36 (m, 1H), 3.29 - 3.07 (m, 4H), 2.82 (br d, J = 11.6 Hz, 1H), 2.38 - 2.26 (m, 1H), 2.17 - 1.74 (m, 3H).
   LCMS (ESI): m/z 340.1 [M+H]⁺
   HPLC: 99.75%
   Chiral HPLC: 98.77%
   Column : CHIRALPAK IG (250*4.6 mm*5 µm)
   Mobile Phase : A: n-Hexane
   Mobile Phase : B: IPA
   A : B :: 50 : 50; Flow rate : 1.0 mL/min
   Retention time : 12.504 min

### Synthesis of ET-111, ET-112, ET-113 & ET-114

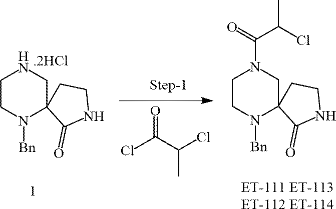

The experimental procedure for the synthesis of compound 1 is captured under ET-103 and ET-104 as racemic mixture of compounds 7A and 7B.

### Synthesis of 6-benzyl-9-(2-chloropropanoyl)-2,6,9-triazaspiro[4.5]decan-1-one (ET-111, ET-112, ET-113 & ET-114)

To a stirring solution of compound 1 (1 g, 3.55 mmol) in CH₂Cl₂ (25 mL) were added Et₃N (1.37 mL, 10.65 mmol) and 2-chloroacetyl chloride (0.38 mL, 4.27 mmol) at -10° C. under nitrogen atmosphere. The reaction mixture was stirred at -10° C. for 2 h. After consumption of the starting material (by TLC), the reaction was diluted with CH₂Cl₂ (20 mL) and washed with water (2 x 5 mL) and brine (2 x 5 mL). The organic layer was dried over anhydrous Na₂SO₄, concentrated under reduced pressure to afford mixture of isomers ET-111, ET-112, ET-113 & ET-114 (920 mg, 83%) as an off white solid. This mixture of isomers were purified by reverse phase column chromatography to obtain ET-111 & ET-112 (450 mg) as an off white solids and another fraction having mixture of ET-113 & ET-114 (380 mg) as an off white solids. Mixture of ET-111, ET-112 (450 mg) was purified by chiral preparative HPLC purification to afford ET-111 (185 mg) as an off white solid and ET-112 (175 mg) as an off white solid. Mixture of ET-113 & ET-114 (380 mg) was purified by chiral preparative HPLC purification to afford ET-113 (175 mg) as an off white solid and ET-114 (180 mg) as an off white solid.

### Et-111

¹H NMR (500 MHz, DMSO-d₆): δ 8.04 - 7.93 (m, 1H), 7.44 - 7.37 (m, 2H), 7.35 - 7.28 (m, 2H), 7.26 - 7.20 (m, 1H), 5.09 (qd, J= 6.4, 17.6 Hz, 1H), 4.12 (dd, J= 1.4, 12.5 Hz, 0.5H), 3.94 - 3.83 (m, 1H), 3.74 (d, J= 13.6 Hz, 0.5H), 3.51 - 3.32 (m, 2H), 3.28 - 3.08 (m, 3H), 2.99 - 2.90 (m, 0.5H), 2.79 (d, J = 12.5 Hz, 0.5H), 2.57 (dt, J = 3.8, 11.9 Hz, 0.5H), 2.27 - 2.01 (m, 3H), 1.77 (dt, J = 5.7, 7.4 Hz, 0.5H), 1.58 - 1.43 (m, 3H)
LC-MS (ESI): m/z 335.9 [M+H]⁺
HPLC: 97.84%
Chiral HPLC: >99.00%
Column : CHIRALPAK IG (250*4.6 mm*5 µm)
Mobile Phase : A: n-Hexane
Mobile Phase : B: IPA
A : B :: 50 : 50; Flow rate : 1.0 mL/min
Retention time : 13.277 min

### Et-112

¹HNMR (500 MHz, DMSO-d₆): δ 8.06 - 7.89 (m, 1H), 7.43 - 7.37 (m, 2H), 7.31 (t, J = 7.5 Hz, 2H), 7.26 - 7.20 (m, 1H), 5.17 - 5.00 (m, 1H), 4.18 - 3.69 (m, 2H), 3.52 - 3.32 (m, 2H), 3.30 -3.05 (m, 3H), 2.98 - 2.75 (m, 1H), 2.60 - 2.51 (m, 1H), 2.27 - 1.72 (m, 3H), 1.56 - 1.42 (m, 3H) LC-MS (ESI): m/z 336.1 [M+H]⁺
HPLC: 99.71%
Chiral HPLC: >99.00%
Column : CHIRALPAK IG (250*4.6 mm*5 µm)
Mobile Phase : A: n-Hexane
Mobile Phase : B: IPA
A : B :: 50 : 50; Flow rate : 1.0 mL/min
Retention time : 17.192 min

### Et-113

¹H NMR (400 MHz, DMSO-d₆): δ 8.09 - 7.92 (m, 1H), 7.46 - 7.36 (m, 2H), 7.32 (t, J = 7.4 Hz, 2H), 7.27 - 7.21 (m, 1H), 5.17 - 4.98 (m, 1H), 4.14 (br d, J= 12.6 Hz, 1H), 3.91 - 3.70 (m, 1H), 3.55 - 3.44 (m, 1H), 3.29 - 3.10 (m, 4H), 2.90 - 2.65 (m, 1H), 2.57 - 2.52 (m, 1H), 2.37 - 2.04 (m, 2H), 1.96 - 1.74 (m, 1H), 1.57 - 1.47 (m, 3H)
LC-MS (ESI): m/z 336.0 [M+H]⁺
HPLC: 99.78%
Chiral HPLC: >99.00%
Column : CHIRALPAK IG (250*4.6 mm*5 µm)
Mobile Phase : A: n-Hexane
Mobile Phase : B: IPA
A : B :: 50 : 50; Flow rate : 1.0 mL/min
Retention time : 10.522 min

### Et-114

¹H NMR (400 MHz, DMSO-d₆): δ 8.09 - 7.93 (m, 1H), 7.43 - 7.37 (m, 2H), 7.31 (t, J = 7.4 Hz, 2H), 7.27 - 7.19 (m, 1H), 5.17 - 4.95 (m, 1H), 4.15 (br d, J= 12.4 Hz, 1H), 3.89 - 3.70 (m, 1H), 3.55 - 3.39 (m, 1H), 3.29 - 3.11 (m, 4H), 2.91 - 2.64 (m, 1H), 2.61 - 2.52 (m, 1H), 2.39 - 2.03 (m, 2H), 1.97 - 1.72 (m, 1H), 1.57 - 1.46 (m, 3 H)
LC-MS (ESI): m/z 336.0 [M+H]⁺
HPLC: 99.91%
Chiral HPLC: 97.55%
Column : CHIRALPAK IG (250*4.6 mm*5 µm)
Mobile Phase : A: n-Hexane
Mobile Phase : B: IPA
A : B :: 50 : 50; Flow rate : 1.0 mL/min
Retention time : 13.245 min

### Synthesis of ET-109 & ET-110

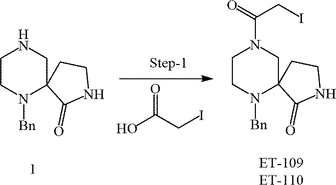

The experimental procedure for the synthesis of compound 1 is captured under ET-103 and ET-104 as racemic mixture of compounds 7A and 7B.

### Synthesis of 6-benzyl-9-(2-iodoacetyl)-2,6,9-triazaspiro[4.5]decan-1-one (ET-109 & ET-110)

To a stirring solution of 2-iodoacetic acid (500 mg, 2.68 mmol) in acetonitrile (10 mL) were added free base compound 1 (793 mg, 3.22 mmol) was dissolved in water (1 mL) at 0° C. and adjusted pH to 7 with aqueous NaHCO₃ solution. Aqueous layer was extracted with CH₂Cl₂ (2 x 1 mL). The organic layer was dried over anhydrous Na₂SO₄, concentrated under reduced pressure. To the free base product in, N-methylmorpholine (0.8 mL, 8.06 mmol) and propylphosphonic anhydride solution (50 wt% in ethyl acetate, 1.7 mL, 5.37 mmol) at room temperature under inert atmosphere and stirred for 16 h. After consumption of the starting material (by TLC), the reaction mixture was diluted with ice water (5 mL) and extracted with EtOAc (2 x 5 mL). The organic layer was washed with brine, dried over anhydrous Na₂SO₄ and concentrated under reduced pressure. The crude was purified by column chromatography by eluting with 5-10% MeOH/ CH₂Cl₂ to afford mixture of isomers (ET-109 & ET-110) (400 mg, 36%) as an off white solid. Mixture of ET-109 & ET-110 (400 mg) was purified by chiral preparative HPLC purification to afford ET-109 (60 mg) as an off white solid and ET-110 (60 mg) as an off white solid.

### Et-109

¹H NMR (400 MHz, DMSO-d₆): δ 8.13 - 7.86 (m, 1H), 7.40 (br t, J= 7.7 Hz, 2H), 7.31 (br t, J = 7.3 Hz, 2H), 7.27 - 7.19 (m, 1H), 4.14 - 3.89 (m, 2H), 3.81 - 3.68 (m, 1H), 3.65 - 3.34 (m, 2H), 3.28 - 3.03 (m, 4H), 2.84 - 2.64 (m, 1H), 2.37 - 2.34 (m, 1H), 2.19 - 2.02 (m, 2H), 1.88 -1.78 (m, 1H)
LC-MS (ESI): m/z 414.1 [M+H]⁺
HPLC: 98.10%
Chiral HPLC: 99.08%
Column : CHIRALPAK IG (250*4.6 mm*5 µm)
Mobile Phase : A: n-Hexane
Mobile Phase : B: IPA
A : B :: 50 : 50; Flow rate : 1.0 mL/min
Retention time : 14.484 min

### Et-110

¹H NMR (400 MHz, DMSO-d₆): δ 8.09 - 7.85 (m, 1H), 7.40 (br t, J= 7.7 Hz, 2H), 7.31 (br t, J = 7.3 Hz, 2H), 7.27 - 7.17 (m, 1H), 4.15 - 3.88 (m, 2H), 3.84 - 3.67 (m, 1H), 3.64 - 3.33 (m, 2H), 3.28 - 3.04 (m, 4H), 2.81 - 2.63 (m, 1H), 2.37 - 2.31 (m, 1H), 2.20 - 2.01 (m, 2H), 1.91 -1.74 (m, 1H)
LC-MS (ESI): m/z 414.3 [M+H]⁺
HPLC: 96.04%
Chiral HPLC: 99.14%
Column : CHIRALPAK IG (250*4.6 mm*5 µm)
Mobile Phase : A: n-Hexane
Mobile Phase : B: IPA
A : B :: 50 : 50; Flow rate : 1.0 mL/min
Retention time : 17.320 min

### Synthesis of ET-170, ET-171, ET-115, ET-116, ET-117, ET-118, ET-119 & ET-120

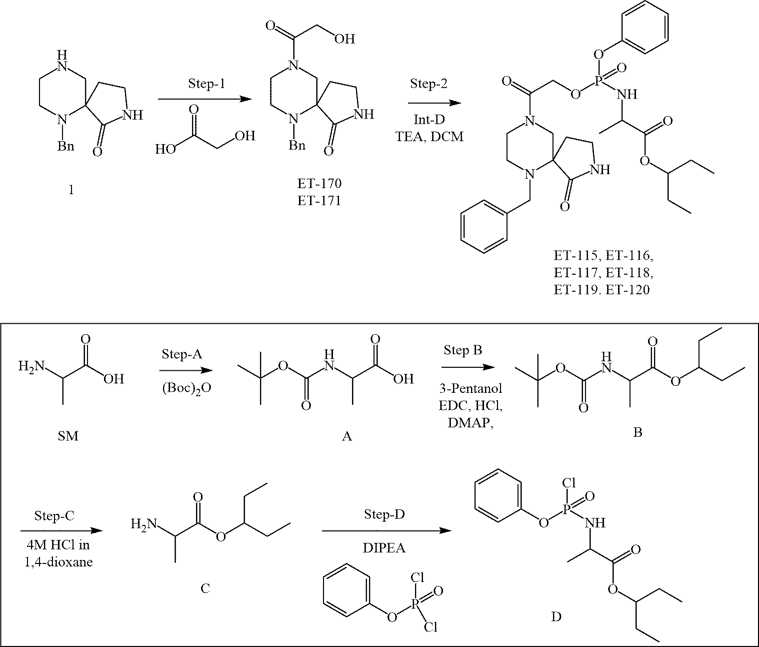

The experimental procedure for the synthesis of compound 1 is captured under ET-103 and ET-104 as racemic mixture of compounds 7A and 7B.

### Synthesis of 6-benzyl-9-(2-hydroxyacetyl)-2,6,9-triazaspiro[4.5]decan-1-one (ET-170 & ET-171)

To a stirring solution of compound 1 (3.5 g, 14.2 mmol) in CH₂Cl₂ (52 mL) were added DIPEA (7.6 mL, 42.8 mmol), 2-hydroxyacetic acid (1.6 g, 21.4 mmol) and HATU (8.1 g, 21.4 mmol) at 0° C. under nitrogen atmosphere. The reaction mixture was stirred at room temperature for 16 h. After consumption of the starting material (by TLC), the reaction was quenched with water (10 mL) and extracted with CH₂Cl₂ (2 x 50 mL). The organic layer was washed with brine, dried over anhydrous Na₂SO₄, concentrated under reduced pressure. The crude material was purified by medium pressure liquid chromatography by eluting 2-3% MeOH/ CH₂Cl₂ to afford mixture of ET-170 & ET-171 (2 g, 46%) as an off white solid. Mixture of ET-170 & ET-171 (2 g) was purified by chiral preparative HPLC purification to afford ET-170 (700 mg) as an off white solid and ET-171 (700 mg) as an off white solid.
ET-170
   ¹H NMR (400 MHz, DMSO-d₆): δ 7.94 (br d, J= 11.9 Hz, 1H), 7.40 (d, J= 7.1 Hz, 2H), 7.34 -7.27 (m, 2H), 7.26 - 7.19 (m, 1H), 4.74 - 4.57 (m, 1H), 4.21 - 3.91 (m, 3H), 3.61 - 3.40 (m, 2H), 3.29 - 3.12 (m, 4H), 3.03 (br t, J = 10.8 Hz, 1H), 2.86 - 2.76 (m, 1H), 2.27 - 2.03 (m, 2H), 2.01 - 1.76 (m, 1H)
   LCMS (ESI): m/z 304.1 [M+H]⁺
   HPLC: 95.59%
   Chiral HPLC: >99.00%
   Column : CHIRALPAK IG (250*4.6 mm*5 µm)
   Mobile Phase : A: n-Hexane
   Mobile Phase : B: IPA
   A : B :: 50 : 50; Flow rate : 1.0 mL/min
   Retention time : 18.915 min
ET-171
   ¹H NMR (400 MHz, DMSO-d₆): δ 7.94 (br d, J= 12.0 Hz, 1H), 7.43 - 7.36 (m, 2H), 7.34 -7.27 (m, 2H), 7.26 - 7.19 (m, 1H), 4.73 - 4.56 (m, 1H), 4.21 - 3.92 (m, 3H), 3.60 - 3.39 (m, 2H), 3.28 - 3.12 (m, 4H), 3.03 (br t, J = 10.8 Hz, 1H), 2.88 - 2.74 (m, 1H), 2.25 - 2.03 (m, 2H), 2.00 - 1.77 (m, 1H)
   LCMS (ESI): m/z 304.1 [M+H]⁺
   HPLC: 96.39%
   Chiral HPLC: 96.89%
   Column : CHIRALPAK IG (250*4.6 mm*5 µm)
   Mobile Phase : A: n-Hexane
   Mobile Phase : B: IPA
   A : B :: 50 : 50; Flow rate : 1.0 mL/min
   Retention time : 25.030 min

### Synthesis of Pentan-3-yl ((2-(6-benzyl-1-oxo-2,6,9-triazaspiro[4.5]decan-9-yl)-2-oxoethoxy)(phenoxy)phosphoryl)alaninate (ET-115)

To a stirring solution of ET-170 (100 mg, 0.33 mmol) in CH₂Cl₂ (5 mL) were added Et₃N (0.14 mL, 0.90 mmol) followed by crude Int-D (439 mg, 1.30 mmol) at 0° C. under inert atmosphere. The reaction mixture was stirred at room temperature for 16 h. After consumption of the starting material (by TLC), the reaction was quenched with water (5 mL) and extracted with CH₂Cl₂ (3 x 50 mL). The organic layer was washed with brine, dried over Na₂SO₄ and concentrated under reduced pressure. The crude material was purified by medium pressure liquid chromatography by eluting with 2-3% MeOH/ CH₂Cl₂ to afford 80 mg, of probably mixture of four unresolved isomers as an off white solid.

Similarly, to a solution of ET-171 (100 mg, 0.33 mmol) in CH₂Cl₂ (5 mL) were added Et₃N (0.14 mL, 0.90 mmol) followed by crude Int-D (439 mg, 1.30 mmol) at 0° C. under inert atmosphere. The reaction mixture was stirred at room temperature for 16 h. After consumption of the starting material (by TLC), the reaction was quenched with water (5 mL) and extracted with CH₂Cl₂ (3 x 50 mL). The organic layer was washed with brine, dried over Na₂SO₄ and concentrated under reduced pressure. The crude material was purified by medium pressure liquid chromatography by eluting with 2-3% MeOH/ CH₂Cl₂ to afford 80 mg of probably mixture of four unresolved isomers) as an off white solid.

10 mg each product from ET-170 and ET-171 reactions were mixed to obtain ET-115 (20 mg, probably mixture of eight unresolved isomers) as an off white solid
ET-115
   ¹H NMR (400 MHz, DMSO-d₆) δ 8.07 - 7.88 (m, 1H), 7.45 - 7.27 (m, 6H), 7.26 - 7.10 (m, 4H), 5.99 - 5.92 (m, 1H), 4.91 - 4.49 (m, 3H), 4.16 - 3.83 (m, 2H), 3.62 - 3.38 (m, 2H), 3.28 -3.13 (m, 3H), 2.87 - 2.70 (m, 1H), 2.22 - 1.94 (m, 3H), 1.62 - 1.38 (m, 5H), 1.26 (br t, J= 7.9 Hz, 4H), 0.86 - 0.73 (m, 6H)
   LCMS (ESI): m/z 601.2 [M+H]⁺
   HPLC: 99.07%
   Chiral HPLC: 34.70%, 16.38% & 48.92%
   Column : Chiralpak AD-H (250 X4.6 mm,5 µm)
   Mobile Phase : A: n-Hexane
   Mobile Phase : B: IPA
   A : B :: 50 : 50; Flow rate : 1.0 mL/min
   Retention time : 12.752 min, 17.652 min & 21.467 min

### Synthesis of Pentan-3-yl ((2-(6-benzyl-1-oxo-2,6,9-triazaspiro[4.5]decan-9-yl)-2-oxoethoxy)(phenoxy)phosphoryl)alaninate (ET-116 & ET-117)

To a stirring solution of ET-170 (100 mg, 0.33 mmol) in CH₂Cl₂ (5 mL) were added Et₃N (0.14 mL, 0.90 mmol) followed by crude Int-D (439 mg, 1.30 mmol) at 0° C. under inert atmosphere. The reaction mixture was stirred at room temperature for 16 h. After consumption of the starting material (by TLC), the reaction was quenched with water (5 mL) and extracted with CH₂Cl₂ (3 x 50 mL). The organic layer was washed with brine, dried over Na₂SO₄ and concentrated under reduced pressure. The crude material was purified by medium pressure liquid chromatography by eluting with 2-3% MeOH/ CH₂Cl₂ followed by chiral preparative HPLC purification afforded ET-116 (18 mg, mixture of isomers which are yet to be resolved and its stereo chemistry to be established) as pale brown sticky solid and mixture of ET-117 (26 mg, mixture of isomers which are yet to be resolved and its stereo chemistry to be established) as pale brown sticky solid.
ET-116
   ¹H NMR (400 MHz, DMSO-d₆): δ 7.94 (br s, 1H), 7.44 - 7.27 (m, 6H), 7.27 - 7.10 (m, 4H), 6.02 - 5.89 (m, 1H), 4.84 - 4.72 (m, 1H), 4.64 (br d, J= 5.3 Hz, 2H), 4.12 - 4.05 (m, 1H), 4.01 - 3.85 (m, 2H), 3.62 - 3.40 (m, 2H), 3.25 - 3.11 (m, 3H), 3.10 - 2.97 (m, 1H), 2.79 (br d, J= 12.9 Hz, 1H), 2.42 (br d, J = 11.0 Hz, 1H), 2.22 - 1.93 (m, 2H), 1.56 - 1.45 (m, 4H), 1.25 (br d, J= 7.5 Hz, 3H), 0.86 - 0.74 (m, 6H)
   LCMS (ESI): m/z 601.2 [M+H]⁺
   HPLC: 96.39%
   Chiral HPLC: 97.53%
   Column : CHIRAL CEL ADH (250*4.6 mm*5 µm)
   Mobile Phase : A: n-Hexane
   Mobile Phase : B: IPA
   A : B :: 50 : 50; Flow rate : 1.0 mL/min
   Retention time : 12.284 min
ET-117
   ¹H NMR (400 MHz, DMSO-d₆): δ 7.94 (br s, 1H), 7.44 - 7.27 (m, 6H), 7.27 - 7.10 (m, 4H), 6.02 - 5.89 (m, 1H), 4.84 - 4.72 (m, 1H), 4.64 (br d, J= 5.3 Hz, 2H), 4.12 - 4.05 (m, 1H), 4.01 - 3.85 (m, 2H), 3.62 - 3.40 (m, 2H), 3.25 - 3.11 (m, 3H), 3.10 - 2.97 (m, 1H), 2.79 (br d, J= 12.9 Hz, 1H), 2.42 (br d, J = 11.0 Hz, 1H), 2.22 - 1.93 (m, 2H), 1.56 - 1.45 (m, 4H), 1.25 (br d, J= 7.5 Hz, 3H), 0.86 - 0.74 (m, 6H)
   LCMS (ESI): m/z 601.2 [M+H]⁺
   HPLC: 99.49%
   Chiral HPLC: >99.00%
   Column : CHIRAL CEL ADH (250*4.6 mm*5 µm)
   Mobile Phase : A: n-Hexane
   Mobile Phase : B: IPA
   A : B :: 50 : 50; Flow rate : 1.0 mL/min
   Retention time : 20.910 min

### Synthesis of Pentan-3-yl ((2-(6-benzyl-1-oxo-2,6,9-triazaspiro[4.5]decan-9-yl)-2-oxoethoxy)(phenoxy)phosphoryl)alaninate (ET-118, ET-119 & ET-120)

To a stirring solution of ET-171 (100 mg, 0.33 mmol) in CH₂Cl₂ (5 mL) were added Et₃N (0.14 mL, 0.90 mmol) followed by crude Int-D (439 mg, 1.30 mmol) at 0° C. under inert atmosphere. The reaction mixture was stirred at room temperature for 16 h. After consumption of the starting material (by TLC), the reaction was quenched with water (5 mL) and extracted with CH₂Cl₂ (3 x 50 mL). The organic layer was washed with brine, dried over Na₂SO₄ and concentrated under reduced pressure. The crude material was purified by medium pressure liquid chromatography by eluting with 2-3% MeOH/ CH₂Cl₂ followed by chiral preparative HPLC purification afforded ET-118 (35 mg, mixture of isomers which are yet to be resolved and its stereo chemistry to be established) as brown sticky solid, ET-119 (31 mg) as brown sticky solid and ET-120 (14 mg) as brown sticky solid.
ET-118
   ¹H NMR (400 MHz, DMSO-d₆): δ 7.96 (br d, J= 16.6 Hz, 1H), 7.44 - 7.27 (m, 6H), 7.26 -7.11 (m, 4H), 6.02 - 5.89 (m, 1H), 4.85 - 4.52 (m, 3H), 4.14 - 3.86 (m, 2H), 3.62 - 3.38 (m, 2H), 3.27 - 3.13 (m, 4H), 3.08 - 2.75 (m, 1H), 2.24 - 1.75 (m, 4H), 1.62 - 1.38 (m, 4H), 1.26 (br t, J= 7.1 Hz, 3H), 0.80 (t, J= 7.4 Hz, 6H)
   LCMS (ESI): m/z 601.3 [M+H]⁺
   HPLC: 98.90%
   Chiral HPLC: 54.06% & 45.94%
   Column : Chiralpak IC (150 X4.6 mm,3 µm)
   Mobile Phase : A: 0.1% TFA in n-Hexane
   Mobile Phase : B: DCM:MEOH (1:1)
   A : B :: 75 : 25; Flow rate : 0.7 mL/min
   Retention time : 8.957 min & 9.128 min
ET-119
   ¹H NMR (400 MHz, DMSO-d₆): δ 7.96 (br d, J= 18.2 Hz, 1H), 7.42 - 7.27 (m, 6H), 7.26 -7.14 (m, 4H), 5.96 (br dd, J= 10.2, 12.6 Hz, 1H), 4.82 - 4.55 (m, 3H), 4.13 - 3.92 (m, 2H), 3.61 - 3.41 (m, 2H), 3.25 - 3.08 (m, 4H), 3.05 - 2.70 (m, 1H), 2.18 - 1.95 (m, 4H), 1.60 - 1.39 (m, 4H), 1.20 - 1.11 (m, 3H), 0.80 (br t, J= 7.0 Hz, 6H)
   LCMS (ESI): m/z 601.3 [M+H]⁺
   HPLC: 88.79%
   Chiral HPLC: >99.00%
   Column : Chiralpak IC (150 X4.6 mm,3 µm)
   Mobile Phase : A: 0.1% TFA in n-Hexane
   Mobile Phase : B: DCM:MEOH (1:1)
   A : B :: 75 : 25; Flow rate : 0.7 mL/min
   Retention time : 12.985 min
ET-120
   ¹H NMR (400 MHz, DMSO-d₆): δ 8.00 - 7.88 (m, 1H), 7.41 - 7.25 (m, 6H), 7.24 - 7.11 (m, 4H), 6.01 - 5.86 (m, 1H), 4.84 - 4.70 (m, 1H), 4.68 - 4.54 (m, 2H), 4.10 - 4.01 (m, 1H), 4.00 -3.84 (m, 1H), 3.59 - 3.38 (m, 2H), 3.25 - 3.08 (m, 3H), 3.07 - 2.73 (m, 2H), 2.18 - 1.75 (m, 4H), 1.57 - 1.37 (m, 4H), 1.23 (br d, J= 7.2 Hz, 3H), 0.85 - 0.71 (m, 6H)
   LCMS (ESI): m/z 601.2 [M+H]⁺
   HPLC: 93.26%
   Chiral HPLC: 97.95%
   Column : Chiralpak IC (150 X4.6 mm,3 µm)
   Mobile Phase : A: 0.1% TFA in n-Hexane
   Mobile Phase : B: DCM:MEOH (1:1)
   A : B :: 75 : 25; Flow rate : 0.7 mL/min
   Retention time : 16.862 min

### Intermediate Preparation

### Synthesis of (Tert-Butoxycarbonyl)Alanine (A)

To a stirring solution of DL-Alanine (10 g, 112.3 mmol) in 1,4-dioxane: water (200 mL, 1:1) was added NaHCO₃ (28.3 g, 337.0 mmol) at 0° C. After stirring for 10 min, Boc₂O (30.8 mL, 134.8 mmol) was added drop wise at 0° C. The reaction mixture was brought to room temperature and stirred for 16 h. After consumption of the starting material (by TLC), the reaction mixture was diluted with water (100 mL) and washed with Et2O (2 x 100 mL). Aqueous layer pH was adjusted to 2 with 6N HCl solution and extracted with EtOAc (3 x 100 mL). The organic layer was dried over Na₂SO₄ and concentrated under reduced pressure to afford Int-A (18 g, 85%) as white solid.
¹H NMR (400 MHz, DMSO-d₆): δ 12.37 (br s, 1H), 7.08 (br d, J = 7.6 Hz, 1H), 3.92 (br t, J= 7.4 Hz, 1H), 1.37 (s, 9H), 1.21 (d, J = 7.3 Hz, 3H)
LC-MS (ESI): m/z 134.2 [M-^{t}Bu+H]⁺

### Synthesis of Pentan-3-yl (Tert-Butoxycarbonyl)Alaninate (B)

To a stirring solution of Int-A (5 g, 26.4 mmol) in CH₂Cl₂ (100 mL) were added EDCI.HCl (7.5 g, 39.6 mmol), DMAP (645 mg, 5.29 mmol) and 3-pentanol (2.7 g, 31.7 mmol) and at 0° C. under inert atmosphere. The reaction mixture was brought to room temperature and stirred for 4 h. After consumption of the starting material (by TLC), the reaction mixture was diluted with CH₂Cl₂ (100 mL) and washed with water (3 x 50 mL) and brine (2 x 10 mL). The organic layer was dried over anhydrous Na₂SO₄ and concentrated under reduced pressure. The crude material was purified by medium pressure liquid chromatography by eluting with 10-20% EtOAc/ hexane to afford Int-B (5 g, 73%) as white semi solid.
¹H NMR (400 MHz, DMSO-d₆): δ 7.23 (br d, J = 7.3 Hz, 1H), 4.64 (tt, J = 4.9, 7.4 Hz, 1H), 3.96 (quin, J= 7.3 Hz, 1H), 1.62 - 1.30 (m, 13H), 1.24 (d, J= 7.4 Hz, 3H), 0.82 (q, J= 7.3 Hz, 6H)
LC-MS (ESI): m/z 204.0 [M-^{t}Bu+H]⁺

### Synthesis of Pentan-3-YL Alaninate (C)

To a stirring solution of Int-B (5 g, 19.3 mmol) in CH₂Cl₂ (20 mL) was added 4N HCl in 1,4-dioxane (10 mL) at 0° C. under inert atmosphere. The reaction mixture was stirred at room temperature for 16 h. After consumption of the starting material (by TLC), volatiles were evaporated under reduced pressure. The crude compound was triturated with Et₂O (10 mL) and dried under vacuum. Obtained solid was dissolved in water, pH was adjusted to 7 with aqueous NaHCO₃ solution and extracted with EtOAc (3 x 10 mL). The organic layer was dried over anhydrous Na₂SO₄ and concentrated under reduced pressure to afford Int-C (2.6 g, 86%) as pale brown liquid.

¹H NMR (400 MHz, DMSO-d₆): δ 4.71 (tt, J= 5.0, 7.4 Hz, 1H), 3.45 (q, J= 7.0 Hz, 1H), 1.84 (br s, 2H), 1.70 - 1.45 (m, 4H), 1.24 (d, J= 7.0 Hz, 3H), 0.89 (td, J= 2.6, 7.4 Hz, 6H) LC-MS (ESI): m/z 160.5 [M+H]⁺

### Synthesis of Pentan-3-YL (Chloro(Phenoxy)Phosphoryl)Alaninate (D)

To a stirring solution of Int-C (500 mg, 3.14 mmol) in CH₂Cl₂ (5 mL) were added DIPEA (1.6 mL, 9.43 mmol) at -40° C. under inert atmosphere. After stirring for 5 min, phenyl phosphorodichloridate (788 mg, 3.77 mmol) and continued for 30 min. After consumption of the starting material (by TLC), the reaction was quenched with ice water (5 mL) and extracted with CH₂Cl₂ (3 x 5 mL). The organic layer was dried over Na₂SO₄ and concentrated under reduced pressure to afford Int-D (100 mg, crude) as brown liquid. The crude was taken to next step without any further purification.

### B. Biological Activity of Compounds

### 3CL Protease (SARS-CoV-2 or COVID-19) Assay Study

A study was conducted to assess the inhibitory activity of compounds of the present disclosure.

### Assay Protocol

The assay was performed using 3CL Protease, MBP-tagged (SARS-CoV-2) Assay kit from BPS Biosciences (Catalog #79955-2). The Assay kit is a FRET based assay, where 3CL protease cleaves the fluorescent substrate (substrate details are not provided by the kit manufacturer). Briefly, 2.5 µl (5X concentration) of the compounds diluted in assay buffer and 7.5 µl (10 ng/µl, 75 ng/reaction) of enzyme were added into 384-well black, low binding microtiter plate (round bottom) plates and preincubated for 30 min at room temperature with slow shaking. 2.5 µl of substrate solution was added to each well (final concentration 50 µM) and plate was incubated at room temperature for 4 hours. The assay was performed in duplicate. Fluorescence intensity (excitation at a wavelength 360 nm and detection of emission at a wavelength 460 nm) was measured using Perkin Elmer Envision plate reader. IC₅₀ was calculated using GraphPad Prism to assess the inhibition at different inhibitor concentrations.

### Assay Validation

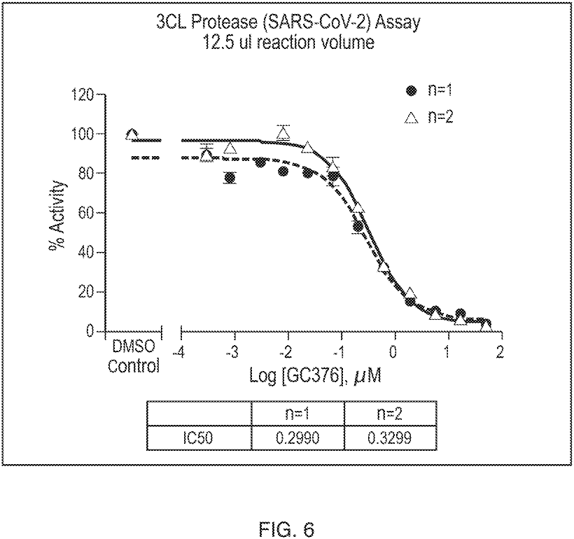

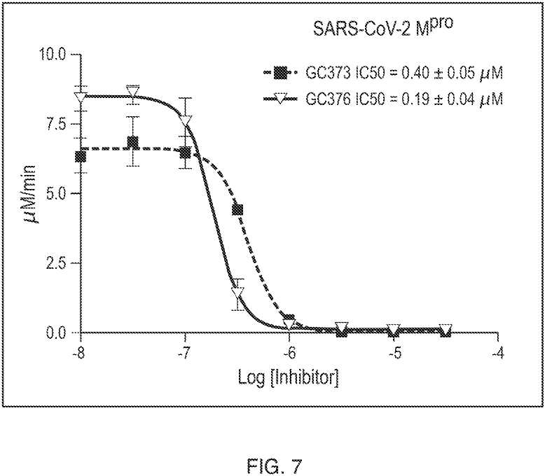

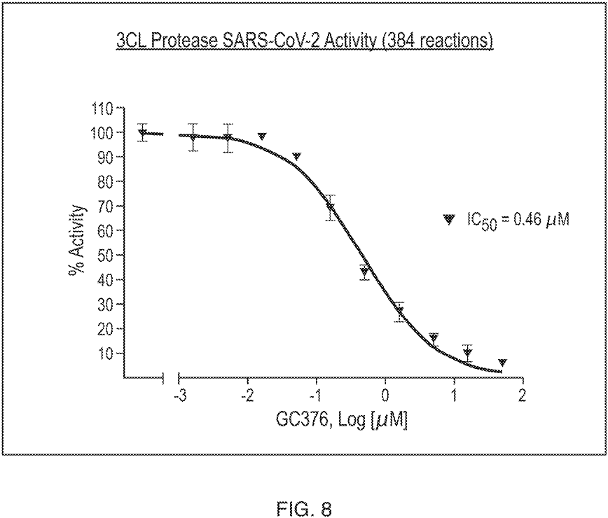

The assay was validated using a tool compound or reference standard GC376 provided in the kit. The assay was validated by two independent experiments (N=2) on different days. GC376 showed IC₅₀ of 0.29 µM (N=1) and 0.33 µM (N=2), as shown in FIG. 6, which is in line with reported values, as shown in FIG. 7 and FIG. 8.

### Compound Study

10 point assay [dose response curve (DRC)] was performed with the compounds (at concentrations 30, 10, 3.3, 1.1, 0.4, 0.1, 0.041, 0.014, 0.005 & 0.002 µM) and tool compound GC376 (at concentrations 50, 16.7, 5.6, 1.9, 0.6, 0.2, 0.069, 0.023, 0.008, 0.003, 0.0008 & 0.0003 µM). Results of the compounds are represented as % activity at tested concentrations. IC₅₀ was calculated using GraphPad Prism.

### Study Results

ET-104, ET-108, ET-110, and ET-103, showed approximately 92%, 84%, 47%, and 44% inhibition, respectively, at 30 µM concentration. ES-319 and ES-320 are found to be inactive at tested concentrations. ET-104 showed an IC₅₀ value of 11.50 µM, and ET-108 showed an IC₅₀ value of 6.00 µM. GC376 showed IC₅₀ of 0.35 µM which is in line with earlier reported values. Table 4 summarizes the COVID-19 3CL Protease inhibitory activity at 30 µM of compounds of the present disclosure.

TABLE 4

| Compound | % Inhibition at 30 µM |
|---|---|
| ES-319 | 0 |
| ES-320 | 0 |
| ET-103 | 44 |
| ET-115 | 31 |
| ET-116 | 28 |
| ET-117 | 15 |
| ET-118 | 34 |
| ET-119 | 34 |
| ET-120 | 30 |
| ET-111 | 0 |
| ET-112 | 0 |
| ET-113 | 0 |
| ET-114 | 0 |
| ET-104 | 92 |
| ET-107 | 38 |
| ET-108 | 84 |
| ET-109 | 36 |
| ET-110 | 47 |

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein

What is claimed is:

1. A method of ameliorating or treating a viral infection in a subject in need thereof, comprising administering to the subject a therapeutically effective amount of a compound of Formula (A), or a pharmaceutically acceptable salt and/or a stereoisomer thereof, wherein Formula (A) is: 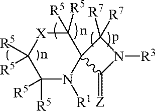
wherein:
X is NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁-C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of —C(O)R³¹, —C(S)R³¹, —C(NH)R³¹ and —C(O)OR³²;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl methyl, and CF₃;
R³¹ is C₁—C₆alkyl, wherein C₁—C₆ alkyl is substituted by one, two or three substituents each independently selected from hydroxyl, S(O)₂—C₁—C₃alkyl, halogen and —OP(O)(R⁴¹R⁴²);
R³² is C₁—C₆alkyl;
R⁴¹ is selected from the group consisting of C₁—C₆alkyl, —C₃—C₆cycloalkyl, and -OR⁴³, wherein R⁴³ is selected from the group consisting of H, C₁—C₆alkyl, —C₃—C₆cycloalkyl, phenyl and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl);
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano and halogen;
R⁷ is independently selected for each occurrence from the group consisting of H, C₁—C₆ alkyl, phenyl and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 2;
n is, for each occurrence, 1; and
w is independently, for each occurrence, 0, 1 or 2.

2. A method of ameliorating or treating a viral infection in a subject in need thereof, comprising administering to the subject a therapeutically effective amount of a compound of Formula (A), or a pharmaceutically acceptable salt and/or a stereoisomer thereof, wherein Formula (A) is: 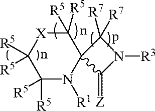
wherein:
X is O or NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl methyl, and CF₃;
R² is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl methyl, and CF₃;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl methyl, and CF₃;
R³¹ and R³² are each independently selected from the group consisting of H, C₁—C₆alkyl, —C₃—C₆cycloalkyl, and phenyl, wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl, halogen and —O—P(O)(R⁴¹R⁴²); and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl, and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁-C₆alkyl);
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano, and halogen;
R⁷ is independently selected for each occurrence from the group consisting of H, C₁—C₆ alkyl, phenyl, and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 1 or 2;
n is independently, for each occurrence, 0, 1 or 2; and
w is independently, for each occurrence, 0, 1 or 2.

3. The method of claim 1 or 2, wherein for Formula (A), R⁵, at each occurrence, is H.

4. The method of any one of claims 1-3, wherein for Formula (A), R⁷, at each occurrence, is H.

5. The method of any one of claims 1-4, wherein for Formula (A), at least one of R¹, R² and R³, independently is —C(O)(C₁—C₆alkyl)X′, wherein X′ is a halogen.

6. The method of any one of claims 1-5, wherein for Formula (A), Z is O.

7. The method of claim 5, wherein for Formula (A), X′ is Br, Cl, F. or I.

8. The method of any one of claims 1-4, wherein for Formula (A), at least one of R¹, R² and R³, independently is —C(O)(C₁—C₆alkyl)X′, wherein X′ is —O—P(O)(R⁴¹R⁴²), wherein R⁴¹ is selected from —O(C₁—C₆alkyl) and —O—phenyl, and R⁴² is —NH(C₁—C₆alkyl) optionally substituted by —C(O)—O(C₁—C₆alkyl).

9. The method of claim 8, wherein for Formula (A), Z is O.

10. The method of claim 8 or 9, wherein for Formula (A), X′ is selected from the group consisting of: 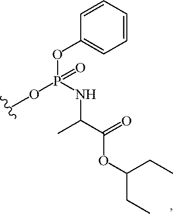 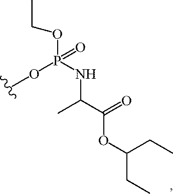 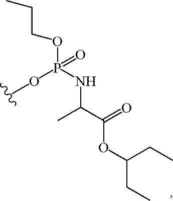 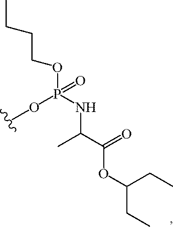 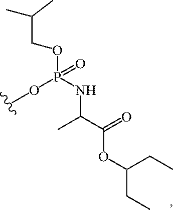 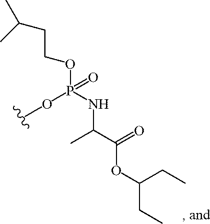 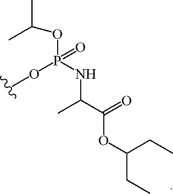
.

11. The method of claim 2, wherein for Formula (A), n, for each occurrence is 1.

12. The method of claim 2, wherein for Formula (A), p is 1.

13. The method of any one of claims 1-12, wherein for Formula (A), R¹ is H.

14. The method of any one of claims 1-12, wherein for Formula (A), R¹ is —C₁alkyl-phenyl, wherein the phenyl may optionally be substituted by one, two or three halogens.

15. The method of claim 2, wherein for Formula (A), X is NR².

16. The method of any one of claims 1-7, 11, 12 and 15, wherein for Formula (A), R² is —C(O)(C₁—C₆alkyl)X′, wherein X′ is a halogen.

17. The method of any one of claims 1-7, 11, 12 and 15, wherein for Formula (A), R² is —C(O)(CH₂)X′, wherein X′ is a halogen.

18. The method of claim 16 or 17, wherein X′ is Br, Cl, F. or I.

19. The method of any one of claims 1-18, wherein for Formula (A), R³ is C₁—C₂alkyl, optionally substituted by one or two substituents each independently selected from phenyl and halogen; and phenyl, independently for each occurrence, is optionally substituted by one, two or three substituents each independently selected from hydroxyl, halogen, —C(O)—C₁—C₃alkyl, methyl, and CF₃.

20. The method of any one of claims 1-18, wherein for Formula (A), R³ is —CH₂—phenyl, wherein phenyl is optionally substituted by one, two or three substituents each independently selected from hydroxyl, halogen, —C(O)—C₁—C₃alkyl, methyl, and CF₃.

21. The method of any one of claims 1-18, wherein for Formula (A), R³ is H.

22. The method of claim 1, wherein Formula (A) is: 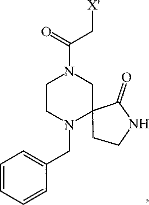
wherein X′ is Br, Cl, or F.

23. The method of claim 1, wherein Formula (A) is: 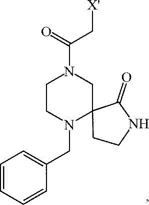
wherein X′ is I or —O—P(O)(R⁴¹R⁴²), wherein R⁴¹ is selected from the group consisting of C₁C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl).

24. The method of claim 23, wherein X′ is selected from the group consisting of 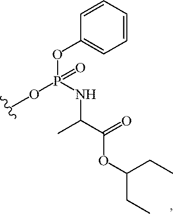 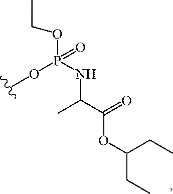 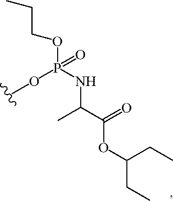 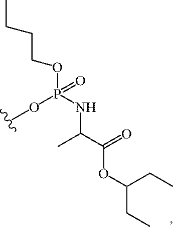 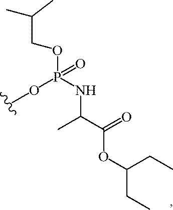 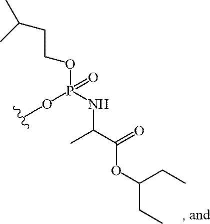 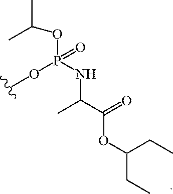
.

25. The method of claim 1, wherein for Formula (A), the compound has the Formula (A-I): 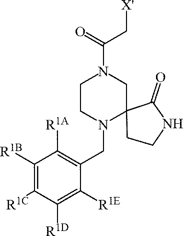
wherein:
X′ is a halogen; and
one, two or three of R^{1A}, R^{1B}, R^{1C}, R^{1D}, and R^{1E} are optionally each independently selected from the group consisting of hydroxyl, halogen, —C(O)—O—C₁—C₃alkyl, —C(O)—C₁—C₃alkyl, methyl, and CF₃.

26. The method of claim 25, wherein for Formula (A-I), the compound is selected from the group consisting of a compound having Formula (A-II); a compound having Formula (A-III); and a compound having Formula (A-IV), wherein:
Formula (A-II) is: 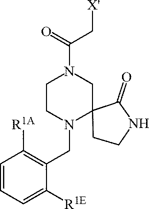
wherein:
X′ is a halogen; and
R^{1A} and R^{1E} are optionally each independently selected from the group consisting of hydroxyl, halogen, —C(O)—O—C₁—C₃alkyl, —C(O)—C₁—C₃alkyl, methyl, and CF₃;
Formula (A-III) is: 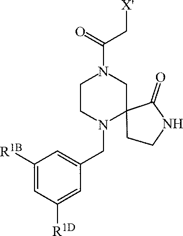
wherein:
X′ is a halogen; and
R^{1B} and R^{1D} are optionally each independently selected from the group consisting of hydroxyl, halogen, —C(O)—O—C₁—C₃alkyl, —C(O)—C₁—C₃alkyl, methyl, and CF₃; and
Formula (A-IV) is: 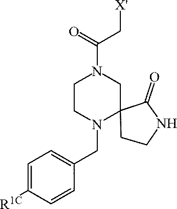
wherein:
X′ is a halogen; and
R^{1C} is optionally selected from the group consisting of hydroxyl, halogen, —C(O)—O—C₁—C₃alkyl, —C(O)—C₁—C₃alkyl, methyl, and CF₃.

27. The method of claim 25, wherein for Formula (A-I), the compound is selected from the group consisting of a compound having Formula (A-V); and a compound having Formula (A-VI), wherein:
Formula (A-V) is: 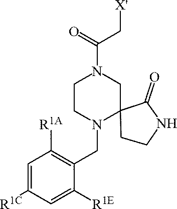
wherein:
X′ is a halogen; and
R^{1A}, R^{1C}, and R^{1E} are optionally each independently selected from the group consisting of hydroxyl, halogen, —C(O)—O—C₁—C₃alkyl, —C(O)—C₁—C₃alkyl, methyl, and CF₃; and
Formula (A-VI) is: 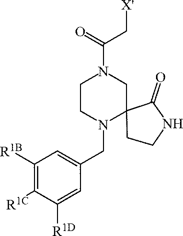
wherein:
X′ is a halogen; and
R^{1B}, R^{1C}, and R^{1D} are optionally each independently selected from the group consisting of hydroxyl, halogen, —C(O)—O—C₁—C₃alkyl, —C(O)—C₁—C₃alkyl, methyl, and CF₃.

28. The method of claim 1, wherein for Formula (A), the compound has the Formula (A-I): 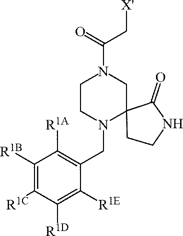
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
one, two or three of R^{1A}, R^{1B}, R^{1C}, R^{1D}, and R^{1E} are optionally each independently selected from the group consisting of H, hydroxyl, halogen, —C(O)—O—C₁—C₃alkyl, —C(O)—C₁—C₃alkyl, methyl, and CF₃.

29. The method of claim 28, wherein for Formula (A-I), the compound is selected from the group consisting of a compound having Formula (A-II); a compound having Formula (A-III); and a compound having Formula (A-IV), wherein:
Formula (A-II) is: 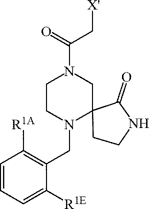
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
R^{1A} and R^{1E} are optionally each independently selected from the group consisting of hydroxyl, halogen, —C(O)—O—C₁—C₃alkyl, —C(O)—C₁—C₃alkyl, methyl, and CF₃;
Formula (A-III) is: 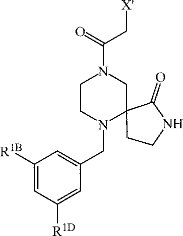
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
R^{1B} and R^{1D} are optionally each independently selected from the group consisting of
hydroxyl, halogen, —C(O)—O—C₁—C₃alkyl, —C(O)—C₁—C₃alkyl, methyl, and CF₃; and
Formula (A-IV) is: 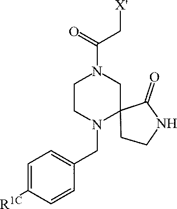
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁-C₆alkyl, -C₃-C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
R^{1C} is optionally selected from the group consisting of hydroxyl, halogen, —C(O)—O—C₁—C₃alkyl, —C(O)—C₁—C₃alkyl, methyl, and CF₃.

30. The method of claim 28, wherein for Formula (A-I), the compound is selected from the group consisting of a compound having Formula (A-V); and a compound having Formula (A-VI), wherein:
Formula (A-V) is: 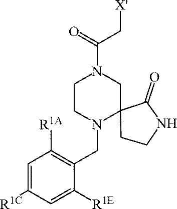
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
R^{1A}, R^{1C}, and R^{1E} are optionally each independently selected from the group consisting of hydroxyl, halogen, —C(O)—O—C₁—C₃alkyl, —C(O)—C₁—C₃alkyl, methyl, and CF₃; and
Formula (A-VI) is: 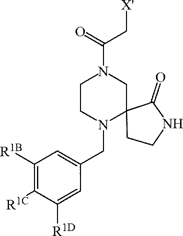
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
R^{1B}, R^{1C}, and R^{1D} are optionally each independently selected from the group consisting of hydroxyl, halogen, —C(O)—O—C₁—C₃alkyl, —C(O)—C₁—C₃alkyl, methyl, and CF₃.

31. A method of ameliorating or treating a viral infection in a subject in need thereof, comprising administering to the subject a therapeutically effective amount of a compound of Formula (A-Ia), or a pharmaceutically acceptable salt and/or a stereoisomer thereof, wherein Formula (A-Ia): 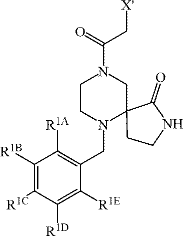
wherein:
X′ is a halogen; and
one, two, three or four of R^{1A}, R^{1B}, R^{1C}, R^{1D}, and R^{1E} are optionally each independently selected from the group consisting of hydroxyl, halogen, —C—O—C₁—C₃alkyl, —C(O)—O—C₁C₃alkyl, —C(O)—C₁—C₃alkyl, methyl, and CF₃.

32. A method of ameliorating or treating a viral infection in a subject in need thereof, comprising administering to the subject a therapeutically effective amount of a compound of Formula (A-Ia-I), or a pharmaceutically acceptable salt and/or a stereoisomer thereof, wherein Formula (A-Ia-I): 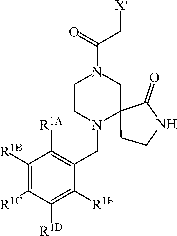
wherein:
X′ is —O—P(O)(R⁴¹R⁴²), wherein R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl; and R⁴² is selected from the group consisting of —NH₂, —NH(C₁—C₆alkyl), and —N(C₁—C₆alkyl)₂, wherein the alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl); and
one, two, three or four of R^{1A}, R^{1B}, R^{1C}, R^{1D}, and R^{1E} are optionally each independently selected from the group consisting of hydroxyl, halogen, —C—O—C₁—C₃alkyl, —C(O)—O—C₁C₃alkyl, —C(O)—C₁—C₃alkyl, methyl, and CF₃.

33. The method of any one of claims 1-32, wherein the viral infection is from a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebola virus, a morbillivirus, an enterovirus, an orthopneumovirus, a lentivirus, arenovirus, a herpes virus, and a hepatovirus.

34. The method of any one of claims 1-33, wherein the viral infection is a coronavirus infection.

35. A conjugate represented by: 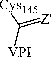
wherein Cys₁₄₅ is cysteine at position 145 or equivalent active site cysteine on Mpro;
Z′ is O, S or NH; and
VPI is a viral protease inhibitor.

36. A conjugate represented by: 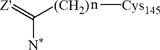
wherein:
Cys₁₄₅ is cysteine at position 145 or equivalent active site cysteine on Mpro;
Z′ is O, S or NH;
n is independently, for each occurrence, 0, 1 or 2; and
N* is a ring nitrogen of a compound, or a pharmaceutically acceptable salt and/or a stereoisomer thereof, wherein N* comprises the compound, or a pharmaceutically acceptable salt and/or a stereoisomer thereof, and the compound is a compound having Formula (A).

37. A conjugate represented by: 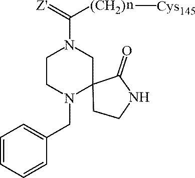
wherein Z′ is O, S or NH; and
n is independently, for each occurrence, 0, 1 or 2.

38. The conjugate of claim 37, wherein Z′ is O.

39. The conjugate of claim 37 or 38, wherein n is 1.

40. A conjugate represented by: 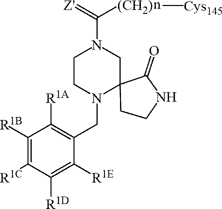
wherein Z′ is O, S or NH;
n is independently, for each occurrence, 0, 1 or 2; and
one, two or three of R^{1A}, R^{1B}, R^{1C}, R^{1D}, and R^{1E} are optionally each independently selected from the group consisting of hydroxyl, halogen, —C(O)—O—C₁—C₃alkyl, —C(O)—C₁—C₃alkyl, methyl, and CF₃.

41. A compound of Formula (A), or a pharmaceutically acceptable salt and/or a stereoisomer thereof, wherein Formula (A) is: 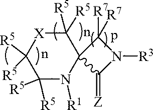
wherein:
X is NR²;
Z is O, S or NH;
R¹ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R² is selected from the group consisting of —C(O)R³¹, —C(S)R³¹, —C(NH)R³¹ and —C(O)OR³²;
R³ is selected from the group consisting of H, C₁—C₆alkyl, phenyl, —C(O)R³¹, —C(S)R³¹, —C(NH)R³¹ and —C(O)OR³², wherein C₁—C₆ alkyl is optionally substituted by one, two or three substituents each independently selected from —C(O)NR^{a}R^{b}, —NR^{a}R^{b}, hydroxyl, S(O)_{w}—C₁—C₃alkyl, SH, phenyl and halogen; and phenyl, independently for each occurrence, is optionally substituted by one, two or three substituents each independently selected from hydroxyl, halogen, —C(O)—O—C₁—C₃alkyl, —C(O)—C₁—C₃alkyl, methyl, and CF₃;
R³¹ is C₁—C₆alkyl, wherein C₁—C₆ alkyl is substituted by one, two or three substituents each independently selected from hydroxyl, S(O)₂—C₁—C₃alkyl, halogen and —O—P(O)(R⁴¹R⁴²);
R³² is C₁—C₆alkyl;
R⁴¹ is selected from the group consisting of C₁—C₆alkyl, —C₃—C₆cycloalkyl, and —OR⁴³, wherein R⁴³ is selected from the group consisting of H, C₁—C₆alkyl, —C₃—C₆cycloalkyl, phenyl and naphthyl;
R⁴² is selected from the group consisting of —NH₂, —NH(C₁—C₆alkyl), and —N(C₁—C₆alkyl)₂, wherein the C₁—C₆alkyl is optionally substituted by one, two or three substituents each independently selected from oxo, hydroxyl, halogen, C₃—C₆cycloalkyl, C₁—C₆alkoxy, —C(O)—(C₁—C₆ alkyl), and —C(O)—O(C₁—C₆alkyl);
R⁵ is independently selected for each occurrence from the group consisting of H, C₁—C₆alkyl, —C₁—C₃alkoxy, —S(O)_{w}—C₁—C₃alkyl, — NR^{a}R^{b}, cyano and halogen;
R⁷ is independently selected for each occurrence from the group consisting of H, C₁—C₆ alkyl, phenyl and halogen;
R^{a} and R^{b} are each independently for each occurrence selected from the group consisting of H, C₁—C₃alkyl, and phenyl, or R^{a} and R^{b} taken together with the nitrogen to which they are attached form a 4-6 membered heterocyclic ring;
p is 2;
n is, for each occurrence, 1; and
w is independently, for each occurrence, 0, 1 or 2.

42. The compound of claim 41, wherein R¹ is C₁alkyl-phenyl, wherein phenyl is optionally substituted by one, two or three substituents each independently selected from hydroxyl, halogen, methyl, and CF₃.
